(19) United States
(12) Patent Application Publication
(10) Pub. No.: US 2012/0035237 A1
(43) Pub. Date: Feb. 9, 2012

(54) PYRAZOLE AND TRIAZOLE CARBOXAMIDES AS CRAC CHANN EL INHIBITORS

(76) Inventors: Diane Mary Coe, Hertfordshire (GB); Anthony William James Cooper, Hertfordshire (GB); Paul Martin Gore, Hertfordshire (GB); David House, Hertfordshire (GB); Stefan Senger, Hertfordshire (GB); Sadie Vile, Essex (GB)

(21) Appl. No.: 13/265,031

(22) PCT Filed: Apr. 22, 2010
(86) PCT No.: PCT/EP10/55318

§ 371 Date: Oct. 18, 2011

Related U.S. Application Data
(60) Provisional application No. 61/172,277, filed on Apr. 24, 2009.

(51) Int. Cl. A61K 31/415; A61P 11/00; A61K 31/4192; A61P 11/06; C07D 231/12; C07D 249/04

(52) U.S. Cl. 514/406; 548/374.1; 548/255; 514/359

(57) ABSTRACT

The present invention relates to amide compounds, processes for their preparation, intermediates usable in these processes, pharmaceutical compositions containing these compounds and to their use in therapy.

### FIELD OF THE INVENTION

The present invention relates to amide compounds, processes for their preparation, intermediates usable in these processes, pharmaceutical compositions containing these compounds and to their use in therapy. More particularly the present invention relates to pyrazole or triazole amide derivatives and their use in the treatment of a number of diseases, conditions or disorders such as allergic disorders, inflammatory disorders and disorders of the immune system.

### BACKGROUND OF THE INVENTION

Calcium release activated calcium (CRAC) channels are a subset of store operated channels (SOC) which are opened in response to depletion of intracellular calcium stores and represent the critical point of calcium entry into certain cells such as mast cells and T-cells.

Two proteins have been identified as the essential components for CRAC channel function namely STIM1 (stromal interaction molecule 1), a calcium sensor localised in the endoplasmic reticulum, and ORAI1, a pore subunit of the CRAC channel that is gated by STIM1.

Small molecule inhibitors of the CRAC channel current (hereafter ICRAC inhibitors) are known in the art, their identification and therapeutic potential are described by Derler et al (Expert Opin. Drug Discovery; 2008; Vol. 3(7) pp. 787-800). U.S. Pat. No. 6,958,339 discloses a series of pyrazole derivatives that are said to possess calcium release-activated calcium channel inhibitory activity which are believed to be useful in the treatment of allergic, inflammatory or autoimmune diseases.

### SUMMARY OF THE INVENTION

A class of compounds has been found that are calcium release activated calcium channel (ICRAC) inhibitors.

In a first aspect of the present invention, there is provided a compound of formula (I) or a salt thereof, more particularly to a compound of formula (I) or a pharmaceutically acceptable salt thereof

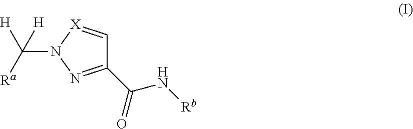

In a second aspect of the present invention, there is provided a pharmaceutical composition comprising a compound of formula (I) or a pharmaceutically acceptable salt thereof and one or more pharmaceutically acceptable carriers, diluents or excipients.

In a third aspect of the present invention, there is provided a compound of formula (I), or a pharmaceutically acceptable salt thereof for use in therapy, in particular in the treatment of diseases or conditions for which an ICRAC inhibitor is indicated.

In a fourth aspect of the present invention, there is provided a method of treating diseases or conditions for which an ICRAC inhibitor is indicated in a subject in need thereof which comprises administering a therapeutically effective amount of compound of formula (I) or a pharmaceutically acceptable salt thereof.

In a fifth aspect of the present invention, there is provided the use of a compound of formula (I), or a pharmaceutically acceptable salt thereof in the manufacture of a medicament for the treatment of diseases or conditions for which an ICRAC inhibitor is indicated.

### DETAILED DESCRIPTION OF THE INVENTION

There is thus provided, in a first aspect of the present invention, a compound of formula (I) or a salt thereof

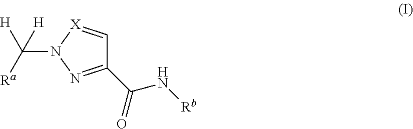

in which

### X is CH or N;

R^{a} is a group of formula (a)

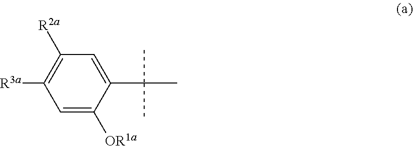

wherein
R^{1a} is a group —(CH₂)ₙ—R⁴
in which n is 0, 1, or 2; and
R⁴ is C₁₋₆alkyl, C₂₋₆alkenyl, C₃₋₇cycloalkyl or aryl;
   R^{2a} and R^{3a} are independently H, halogen or CF₃;
   R^{b} is a group of formula (b)

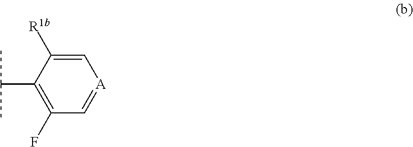

wherein
A is CH or N;
R^{1b} is H, halogen or C₁₋₆alkoxy.

In one embodiment X is CH.

In one embodiment R^{1a} is a C₁₋₆alkyl, C₃₋₇cycloalkyl, or phenyl optionally substituted by one of more substituents e.g. two or three (which may be the same or different) selected from the group consisting of halogen, C₁₋₆alkyl or C₁₋₆alkoxy.

In a further embodiment R^{1a} is a group —(CH₂)ₙ—R⁴ in which n is 1 and R⁴ is O₃₋₇cycloalkyl (such as cycloproyl, cyclobutyl or cyclopentyl) or is phenyl optionally substituted by one or more substituents (e.g. two or three, which may be the same or different) selected from the group consisting of halogen (such as fluoro or chloro), C₁₋₆alkyl (such as methyl) or C₁₋₆alkoxy (such as methoxy, ethoxy or propoxy).

In one embodiment R^{2a} and R^{3a} are both H. In a further embodiment R^{2a} is chloro and R^{3a} is H.

In one embodiment A is CH.

In one embodiment R^{1b} is H, fluoro, chloro or methoxy.

In a further embodiment R^{b} is a group of formula (b) in which A is CH and R^{1b} is fluoro.

While the embodiments for each variable have generally been listed above separately for each variable this invention is intended to include all combinations of embodiments described hereinabove including salts thereof.

Specific compounds according to the invention include Examples 1-61 as described herein or a salt thereof, such as a compound selected from the group consisting of:
1-({5-Chloro-2-[(phenylmethyl)oxy]phenyl}methyl)-N-(2,6-difluorophenyl)-1H-pyrazole-3-carboxamide;
1-({5-Chloro-2-[(2-methylpropyl)oxy]phenyl}methyl)-N-(2,6-difluorophenyl)-1H-pyrazole-3-carboxamide;
1-({5-Chloro-2-[(3-methylbutyl)oxy]phenyl}methyl)-N-(2,6-difluorophenyl)-1H-pyrazole-3-carboxamide;
1-{[2-(Butyloxy)-5-chlorophenyl]methyl}-N-(2,6-difluorophenyl)-1H-pyrazole-3-Carboxamide;
1-({5-Chloro-2-[(cyclopropylmethyl)oxy]phenyl}methyl)-N-(2,6-difluorophenyl)-1H-pyrazole-3-carboxamide;
1-[(5-Chloro-2-{[(2,6-difluorophenyl)methyl]oxy}phenyl)methyl]-N-(2,6-difluorophenyl)-1H-pyrazole-3-carboxamide;
1-{[5-Chloro-2-(propyloxy)phenyl]methyl}-N-(2,6-difluorophenyl)-1H-pyrazole-3-carboxamide;
1-({5-Chloro-2-[(cyclobutylmethyl)oxy]phenyl}methyl)-N-(2,6-difluorophenyl)-1H-pyrazole-3-carboxamide;
N-(2,6-Difluorophenyl)-1-({2-[(phenylmethyl)oxy]phenyl}methyl)-1H-pyrazole-3-carboxamide;
N-(2,6-Difluorophenyl)-1-({2-[(2-methylpropyl)oxy]phenyl}methyl)-1H-pyrazole-3-carboxamide;
1-({2-[(Cyclobutylmethyl)oxy]phenyl}methyl)-N-(2,6-difluorophenyl)-1H-pyrazole-3-carboxamide;
N-(2,6-Difluorophenyl)-1-({2-[(2-methylbutyl)oxy]phenyl}methyl)-1H-pyrazole-3-carboxamide;
N-(2,6-Difluorophenyl)-1-({2-[(3-methyl-2-buten-1-yl)oxy]phenyl}methyl)-1H-pyrazole-3-carboxamide;
1-{[5-Chloro-2-(phenyloxy)phenyl]methyl}-N-(2,6-difluorophenyl)-1H-pyrazole-3-carboxamide;
1-({5-Bromo-2-[(phenylmethyl)oxy]phenyl}methyl)-N-(2,6-difluorophenyl)-1H-pyrazole-3-carboxamide;
N-(2,6-difluorophenyl)-1-{[2-(phenyloxy)phenyl]methyl}-1H-pyrazole-3-carboxamide
1-{[2-(Butyloxy)phenyl]methyl}-N-(3,5-difluoro-4-pyridinyl)-1H-pyrazole-3-carboxamide;
1-{[2-(Butyloxy)phenyl]methyl}-N-(2-chloro-6-fluorophenyl)-1H-pyrazole-3-carboxamide;
2-({5-Chloro-2-[(phenylmethyl)oxy]phenyl}methyl)-N-(2,6-difluorophenyl)-2H-1,2,3-triazole-4-carboxamide;
2-({2-[(Cyclobutylmethyl)oxy]phenyl}methyl)-N-(2,6-difluorophenyl)-2H-1,2,3-triazole-4-carboxamide;
N-(2,6-difluorophenyl)-2-{[2-(phenyloxy)phenyl]methyl}-2H-1,2,3-triazole-4-carboxamide;
2-({5-chloro-2-[(2-methylpropyl)oxy]phenyl}methyl)-N-(2,6-difluorophenyl)-2H-1,2,3-triazole-4-carboxamide;
2-{[2-(Butyloxy)-5-chlorophenyl]methyl}-N-(2,6-difluorophenyl)-2H-1,2,3-triazole-4-carboxamide;
N-(2,6-difluorophenyl)-1-[(2-{[(4-methylphenyl)methyl]oxy}phenyl)methyl]-1H-pyrazole-3-carboxamide;
N-(2,6-difluorophenyl)-1-{[2-({[3-(methyloxy)phenyl]methyl}oxy)phenyl]methyl}-1H-pyrazole-3-carboxamide;
N-(2,6-difluorophenyl)-1-{[2-({[4-(methyloxy)phenyl]methyl}oxy)phenyl]methyl}-1H-pyrazole-3-carboxamide;
N-(2,6-difluorophenyl)-1-{[2-({[4-(ethyloxy)phenyl]methyl}oxy)phenyl]methyl}-1H-pyrazole-3-carboxamide; and
N-(2,6-difluorophenyl)-1-{[2-({[3-(ethyloxy)phenyl]methyl}oxy)phenyl]methyl}-1H-pyrazole-3-carboxamide or a salt thereof.

Throughout the present specification, unless otherwise stated:
the term “halogen” is used to describe a group selected from fluorine, chlorine, bromine or iodine;
the term “C₁₋₆alkyl”, whether alone or used as part of another group, is used to describe a group comprising a linear or branched alkyl group containing from 1 to 6 carbon atoms; examples of such groups include methyl, ethyl, propyl, isopropyl, n-butyl, isobutyl, tert-butyl, pentyl or hexyl;
the term “C₂₋₆alkenyl” is used to describe a linear or branched alkyl group containing from 2 to 6 carbon atoms and having at least one carbon carbon double bond; examples of such group include ethenyl, propenyl and butenyl;
the term “C₃₋₇cycloalkyl” is used to describe a non aromatic carbocyclic ring containing at least three and at most seven carbon atoms. Examples of C₃₋₇cycloalkyl include, but are not limited to cyclopropyl, cyclobutyl, cyclopentyl or cyclohexyl;
the term “C₁₋₆alkoxy” is used to describe a group or a part of the group wherein an oxygen atom is bound to the rest of the molecule and the above mentioned C₁₋₆alkyl group; examples of such groups include methoxy, ethoxy, propoxy, isopropoxy, n-butoxy, isobutoxy, tert-butoxy, pentoxy or hexoxy;
The term “aryl” is used to describe an aromatic hydrocarbon ring such as phenyl and naphthyl. Such aryl groups may be optionally substituted by one of more substituents (e.g. two or three, which may be the same or different) selected from the group consisting of halogen, C₁₋₆alkyl or C₁₋₆alkoxy.

It will be appreciated that the present invention covers compounds of formula (I) as the free base and as salts thereof. Because of their potential use in medicine, salts of the compounds of formula (I) are desirably pharmaceutically acceptable. Suitable pharmaceutically acceptable salts can include acid or base addition salts. As used herein, the term ‘pharmaceutically acceptable salt’ means any pharmaceutically acceptable salt of a compound of formula (I) (in stoichiometric or non-stoichiometric form). For a review on suitable salts see Berge et al., J. Pharm. Sci., 66:1-19, (1977). Typically, a pharmaceutically acceptable salt may be readily prepared by using a desired acid or base as appropriate. The resultant salt may precipitate from solution and be collected by filtration or may be recovered by evaporation of the solvent.

Pharmaceutically acceptable acid addition salts include hydrobromide, hydrochloride, sulfate, nitrate, phosphate, succinate, maleate, acetate, propionate, fumarate, citrate, tartrate, lactate, benzoate, salicylate, glutamate, aspartate, p-toluenesulfonate, benzenesulfonate, methanesulfonate, ethanesulfonate, naphthalenesulfonate (e.g. 2-naphthalenesulfonate) or hexanoate salt.

In one embodiment the compound of formula (I) is in the form of a free base.

The invention encompasses all prodrugs, of the compound of formula (I), which upon administration to the recipient is capable of providing (directly or indirectly) the compound of formula (I), or an active metabolite or residue thereof. Such prodrugs are recognizable to those skilled in the art, without undue experimentation. Nevertheless, reference is made to the teaching of Burger's Medicinal Chemistry and Drug Discovery, 5^{th} Edition, Vol 1: Principles and Practice, which is incorporated herein by reference to the extent of such teaching, and to Rautio et al (Nature Reviews; 2008; Vol. 7, p 255-270).

It will be appreciated that many organic compounds can form complexes with solvents in which they are reacted or from which they are precipitated or crystallized. These complexes are known as “solvates”. For example, a complex with water is known as a “hydrate”. Solvents with high boiling points and/or capable of forming hydrogen bonds such as water, xylene, N-methylpyrrolidinone, methanol and ethanol may be used to form solvates. Methods for identification of solvates include, but are not limited to, NMR and microanalysis. Solvates of the compounds of formula (I) are within the scope of the invention.

The compounds of formula (I) may be in crystalline or amorphous form. Furthermore, some of the crystalline forms of the compounds of formula (I) may exist as polymorphs, which are included within the scope of the present invention. Polymorphic forms of compounds of formula (I) may be characterized and differentiated using a number of conventional analytical techniques, including, but not limited to, X-ray powder diffraction (XRPD) patterns, infrared (IR) spectra, Raman spectra, differential scanning calorimetry (DSC), thermogravimetric analysis (TGA) and solid state nuclear magnetic resonance (SSNMR).

Certain of the compounds described herein may contain one or more chiral atoms so that optical isomers, e.g.—enantiomers or diastereoisomers may be formed. Accordingly, the present invention encompasses isomers of the compounds of formula (I) whether as individual isomers isolated such as to be substantially free of the other isomer (i.e. pure) or as mixtures (i.e. racemates and racemic mixtures). An individual isomer isolated such as to be substantially free of the other isomer (i.e. pure) may be isolated such that less than 10%, particularly less than about 1%, for example less than about 0.1% of the other isomer is present.

Further it will be understood that the present invention encompasses geometric isomers of the compounds of formula (I) including cis and trans configurations (e.g. when R⁴ is C₂₋₆alkenyl) whether as individual isomers isolated such as to be substantially free of the other isomers (i.e. pure) or as mixtures thereof. Thus, for example the present invention encompases an individual isomer isolated such as to be substantially free of the other isomer (i.e. pure) such that less than 10%, for example less than 1% or less than 0.1% of the other isomer is present.

Separation of isomers may be achieved by conventional techniques known to those skilled in the art, e.g. by fractional crystallisation, chromatography or HPLC.

Certain compounds of formula (I) may exist in one of several tautomeric forms. It will be understood that the present invention encompasses all tautomers of the compounds of formula (I) whether as individual tautomers or as mixtures thereof.

It will be appreciated from the foregoing that included within the scope of the invention are solvates, isomers and polymorphic forms of the compounds of formula (I) and salts thereof.

The compounds of the invention may be made by a variety of methods, including standard chemistry. Any previously defined variable will continue to have the previously defined meaning unless otherwise indicated. Illustrative general synthetic methods are set out below and then specific compounds of the invention are prepared in the working Examples.

In a further aspect the present invention provides a process for the preparation of a compound of formula (I) which is a process selected from (a), (b), (c) and (d) in which:

(a) comprises the reaction of a compound of formula (II) or an activated derivative thereof

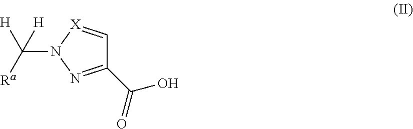

in which R^{a} and X are as defined in formula (I) with a compound of formula (III)

R^{b}—NH₂ (III)

in which R^{b} is as defined in formula (I);
(b) comprises the reaction of a compound of formula (IV)

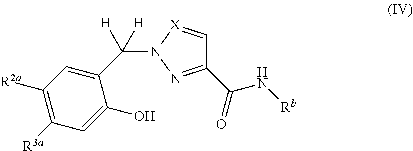

in which R^{b}, R^{2a}, R^{3a} and Xare as defined in formula (I) with a compound of formula (V)

Hal-(CH₂)ₚ—R⁴ (V)

in which p is 1 or 2, R⁴ is as in formula (I) and Hal is a halogen atom;
(c) comprises the reaction of a compound of formula (VI)

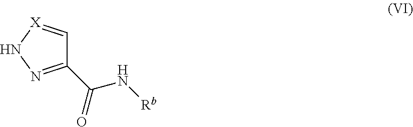

in which R^{b} and X are as defined in formula (I) with a compound of formula (VII)

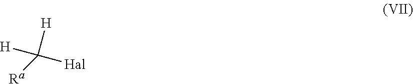

in which R^{a} is as defined in formula (I) and Hal is a halogen atom;
(d) comprises reacting a compound of formula (VIII)

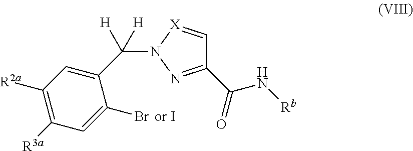

in which R^{b}, R^{2a}, R^{3a} and X are as defined in formula (I) with a compound of formula (IX)

HO—R⁴ (IX)

in which R⁴ is aryl;

### Process (a)

The reaction is typically carried using an activated derivative of a compound of formula (II) such as an acid chloride which is prepared from the corresponding acid by, for example, treating with thionyl chloride. The reaction between an activated compound of formula (II) and a compound of formula (III) is typically carried out in an inert organic solvent such as tetrahydrofuran, dimethylformamide, chloroform or dichloromethane or a mixed organic/aqueous system at ambient or elevated temperature, optionally in the presence of a suitable base e.g. an organic base (such as triethylamine or diisopropylamine), an alkali metal carbonate (such as potassium carbonate) or a alkali metal hydrogen carbonate (such as sodium hydrogen carbonate).

The compounds of formula (II) may be prepared by the representative scheme shown below.

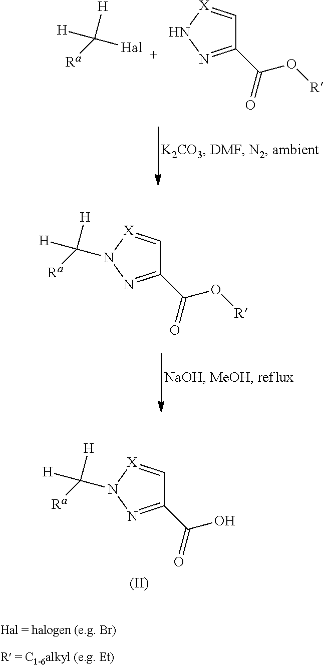

Other compounds of formula (II) and the compounds of formula (III) may be prepared by methods described herein, by analogous methods thereto or are obtainable from commercial sources.

### Process (b)

For compounds of formula (V) a suitable Hal group is bromine or iodine.

The reaction between the compounds of formula (IV) and (V) may be carried out in an inert organic solvent such as dimethylformamide at ambient or elevated temperature, optionally in the presence of a suitable base such as potassium or caesium carbonate or a strong base such as sodium hydride.

The compounds of formula (IV) may be prepared by hydrogenolysis of a compound of formula (I) in which R^{1a} is benzyl using standard methodology such as over palladium on charcoal.

Other compounds of formula (IV) and the compounds of formula (V) may be prepared by methods described herein, by analogous methods thereto or are obtainable from commercial sources.

### Process (c)

For compounds of formula (VII) a suitable Hal group is bromine or iodine.

The alkylation reaction between the compounds of formula (VI) and (VII) may be carried out in an inert organic solvent such as dimethylformamide at ambient or elevated temperature, optionally in the presence of a suitable base such as potassium or caesium carbonate or a strong base such as sodium tert-butoxide.

The compounds of formula (IV) and (V) are prepared by methods described herein, by analogous methods thereto or are obtainable from commercial sources.

### Process (d)

The reaction between the compounds of formula (VIII) and (IX) is carried out in an organic solvent such as dimethylsulfoxide in the presence of a copper catalyst such as copper(I) bromide, a suitable base such as potassium or caesium carbonate and a ligand such as ethyl-2-cyclohexanone carboxylate in a variety of solvents including toluene, dioxane, N,N-dimethylformamide, N,N-dimethylacetamide and dimethylsulfoxide at a temperature in the range 60-160° C., more typically 110° C.

The compounds of formula (VIII) and (IX) are prepared by methods described herein, by analogous methods thereto or are obtainable from commercial sources.

It will be appreciated that in any of the routes (a) to (d) described above, the precise order of the synthetic steps by which the various groups and moieties are introduced into the molecule may be varied. It will be within the skill of the practitioner in the art to ensure that groups or moieties introduced at one stage of the process will not be affected by subsequent transformations and reactions, and to select the order of synthetic steps accordingly. In some instances it may be appropriate to use protecting groups to prevent reactions between one or more groups or moieties. Such procedures are familiar to the those skilled in the art (see, for example, “Protective groups in organic synthesis” by T. W. Greene and P. G. M. Wuts (John Wiley & sons 1999) or “Protecting Groups” by P. J. Kocienski (Georg Thieme Verlag 1994).

It will be further appreciated that novel intermediates described herein form another aspect of the present invention.

The compounds of formula (I) and salts thereof are believed to be calcium release activated calcium channel inhibitors, and thus be potentially useful in the treatment of diseases or conditions for which such a compound is indicated, particularly inflammatory and/or allergic diseases.

The present invention thus provides a compound of formula (I) or a pharmaceutically acceptable salt thereof for use in therapy.

The present invention thus provides a compound of formula (I) or a pharmaceutically acceptable salt thereof for use in the treatment of diseases or conditions for which an ICRAC inhibitor is indicated.

Also provided is the use of a compound of formula (I) or a pharmaceutically acceptable salt thereof in the manufacture of a medicament for the treatment of diseases or conditions for which an ICRAC inhibitor is indicated.

Also provided is a method of treating diseases or conditions for which an ICRAC inhibitor is indicated in a subject in need thereof which comprises administering a therapeutically effective amount of compound of formula (I) or a pharmaceutically acceptable salt thereof.

Suitably the subject in need thereof is a mammal, such as a human.

As used herein, the term “effective amount” means that amount of a drug or pharmaceutical agent that will elicit the biological or medical response of a tissue, system, mammal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

Calcium release activated calcium channel inhibitors (i.e. ICRAC inhibitors) are believed to be indicated in the treatment and/or prophylaxis of a variety of diseases, conditions or disorders in mammals such as humans. These include allergic disorders, inflammatory disorders, disorders of the immune system and conditions in which anti-platelet or anti-thrombotic activity is useful.

Examples of allergic disorders include: rhinitis (such as allergic rhinitis), sinusitis, rhinosinusitis, chronic or recurrent otitis media, drug reactions, insect sting reactions, latex allergy, conjunctivitis, urticaria, anaphylaxis and anaphylactoid reactions, atopic dermatitis and food allergies.

Examples of inflammatory disorders include: inflammatory lung disorders (such as asthma, acute respiratory distress syndrome, chronic obstructive pulmonary disease, bronchiectasis and cystic fibrosis); chronic inflammatory disorders of joints (such as arthritis, rheumatoid arthritis, osteoarthritis and bone diseases associated with increased bone resorption); inflammatory bowel diseases (such as Barrett's oesophagus, ileitis, ulcerative colitis and Crohn's disease); inflammatory disorders of the eye (such as corneal dystrophy, trachoma, uveitis, sympathetic ophthalmitis and endophthalmitis); inflammatory diseases of the kidney (such as glomerulonephritis, nephrosis, nephritic syndrome and IgA nephropathy); inflammatory disorders of the skin (such as psoriasis and eczema); inflammatory diseases of the central nervous system (such as chronic demyelinating diseases of the nervous system, multiple sclerosis, AIDS-related neurodegeneration and Alzheimers disease, infectious meningitis, enceophalomyelitis, Parkinson's disease, Huntington's disease, amyotrophic lateral sclerosis and viral or autoimmune encephalitis); inflammatory diseases of the heart (such as myocarditis and cardiomyopathy, ischemic heart disease and atherosclerosis); other diseases with significant inflammatory components, including tuberculosis; leprosy; rejection of transplants; pre-eclampsia; chronic liver failure; brain and spinal cord trauma and cancer; and conditions where systemic inflammation of the body may also be present (such as septic shock, hemorrhagic or anaphylactic shock or shock induced by cancer chemotherapy).

Examples of disorders of the immune system include: autoimmune diseases of the central and peripheral nervous system (such as multiple sclerosis, myasthenia gravis, Eaton-Lambert Myasthenic syndrome); autoimmune neurophathies (such as Guillain-Barré); autoimmune diseases of the eye (such as autoimmune uveitis); autoimmune diseases of the blood (such as autoimmune haemolytic anemia, pernicious anemia, and autoimmune thrombocytopenia e.g. Idiopathic Thrombocytopaenic Purpura); autoimmune diseases of the vasculature (such as temporal arteritis, anti-phospholipid syndrome, vasculitides e.g. Wegener's granulomatosis and Behcet's disease); autoimmune diseases of the skin (such as alopecia greata, psoriasis, dermatitis herpetiformis, pemphigus vulgaris, bullous pemphigoid and vitiligo); autoimmune disease of the gastrointestinal tract (such as coeliac disease, Crohn's disease, ulcerative colitis, primary biliary cirrhosis and autoimmune hepatitis); autoimmune disorders of the endocrine glands (such as Type1 diabetes mellitus, autoimmune thyroiditis, Grave's disease, Hashimoto's thyroiditis, autoimmune oophoritis and orchitis); autoimmune disorder of the adrenal gland (such as Addisons disease); and multi system autoimmune diseases including connective tissue and musculoskeletal system diseases (such as rheumatoid arthritis, systemic lupus erythematosus, scleroderma, polymyositis, dermatomyositis), spondyloarthropathies (such as ankylosing spondylitis and psoriatic arthritis).

Examples of conditions where anti-platelet or anti-thrombotic activity is useful for treatment and/or prophylaxis include: ischemic heart disease, myocardial infarction, cerebrovascular accident (stroke) and vascular thrombosis (venous, arterial and intra-cardiac).

Further diseases or conditions which may be treated by the compounds of the invention include conditions where mast cells and basophils contribute to pathology, such as mast cell leukaemia, mastocytosis, endometriosis and basophil leukaemia.

The term “diseases or conditions for which an ICRAC is indicated”, is intended to include each of or all of the above disease states.

It is believed that the compounds of formula (I), having ICRAC inhibitory activity, may inhibit mast cell degranulation and/or inhibit T cell activation. Compounds having such activity may be particularly suitable for the treatment of a number of diseases and conditions, for example asthma and rhinitis.

In one embodiment the disease or condition for which an ICRAC inhibitor is indicated is asthma.

In a further embodiment the disease or condition for which an ICRAC inhibitor is indicated is rhinitis

While it is possible that for use in therapy, a compound of formula (I) as well as pharmaceutically acceptable salts thereof may be administered as the raw chemical, it is typical to formulate in a suitable composition comprising one or more pharmaceutically acceptable carriers, diluents or excipients. Such compositions may be prepared using standard procedures.

Thus, there is provided a pharmaceutical composition comprising a compound of formula (I) or a pharmaceutically acceptable salt thereof and one or more pharmaceutically acceptable carriers, diluents or excipients.

Thus there is provided a pharmaceutical composition for the treatment of diseases or conditions in which an ICRAC inhibitor is indicated comprising a compound of formula (I) or a pharmaceutically acceptable salt thereof.

A composition comprising a compound of formula (I) or a pharmaceutically acceptable salt thereof, which may be prepared by admixture, suitably at ambient temperature and atmospheric pressure, may be suitable for topical administration (which includes epicutaneous, inhaled, intranasal or ocular administration), enteral administration (which includes oral or rectal administration) or parenteral administration (such as by injection or infusion). Of interest are pharmaceutical compositions comprising a compound of formula (I) or a pharmaceutically acceptable salt thereof, suitable for topical administration, particularly suitable for intranasal administration.

Generally, compositions may be in the form of solutions or suspensions (aqueous or non-aqueous), tablets, capsules, oral liquid preparations, powders, granules, lozenges, lotions, creams, ointments, gels, foams, reconstitutable powders or suppositories as required by the route of administration.

Generally, the compositions comprising a compound of formula (I) or a pharmaceutically acceptable salt thereof may contain from about 0.1% to 99% (w/w), such as from about 10 to 60% (w/w) (based on the total weight of the composition), of the compound of formula (I) or the pharmaceutically acceptable salt thereof, depending on the route of administration. The dose of the compound used in the treatment of the aforementioned diseases will vary in the usual way with the seriousness of the diseases, the weight of the sufferer, and other similar factors. However, as a general guide, suitable unit doses may be about 0.05 to 1000 mg, for example about 0.05 to 200 mg, and such unit doses may be administered more than once a day, for example two or three times a day or as desired. Such therapy may extend for a number of weeks or months.

Further provided is a pharmaceutical composition for the treatment of a disease or condition for which an ICRAC inhibitor is indicated comprising a compound of formula (I) or prodrug thereof, or a pharmaceutically acceptable salt thereof. In one embodiment there is provided a pharmaceutical composition for the treatment of an allergic disorder (such as rhinitis) or an inflammatory disorder (such as asthma) comprising a compound of formula (I) or prodrug thereof, or a pharmaceutically acceptable salt thereof.

Thus there is provided a pharmaceutical composition comprising 0.05 to 1000 mg of a compound of formula (I) or a pharmaceutically acceptable salt thereof and 0.1 to 2 g of one or more pharamceutically acceptable carriers, diluents and/or excipients.

The proportion of the compound of formula (I) or a pharmaceutically acceptable salt thereof in a topical composition will depend on the precise type of composition to be prepared and the particular route of administration, but will generally be within the range of from about 0.001 to 10% (w/w), based on the total weight of the composition. Generally, however for most types of preparations the proportion used will be within the range of from about 0.005 to 1% (w/w), such as about 0.01 to 1% (w/w), for example about 0.01 to 0.5% (w/w), based on the total weight of the composition. However, in powders for inhalation the proportion used will generally be within the range of from about 0.1 to 5% (w/w), based on the total weight of the composition.

Generally, compositions suitable for intranasal or inhaled administration may conveniently be formulated as aerosols, solutions, suspensions, drops, gels or dry powders, optionally with one or more pharmaceutically acceptable carriers and/or excipients such as aqueous or non-aqueous vehicles, thickening agents, isotonicity adjusting agents, antioxidants and/or preservatives.

For pharmaceutical compositions suitable for intranasal or inhaled administration, the compound of formula (I) or a pharmaceutically acceptable salt thereof may typically be in a particle-size-reduced form, which may be prepared by conventional techniques, for example, micronisation and milling. Generally, the size-reduced (e.g. micronised) compound of formula (I) or a pharmaceutically acceptable salt thereof can be defined by a D₅₀ value of about 0.5 to 10 microns, such as of about 2 to 4 microns (for example as measured using laser diffraction).

In one aspect, pharmaceutical compositions comprising a compound of formula (I) or a pharmaceutically acceptable salt thereof are suitable for intranasal administration. Intranasal compositions comprising a compound of formula (I) or a pharmaceutically acceptable salt thereof may permit the compound(s) to be delivered to all areas of the nasal cavities (the target tissue) and further, may permit the compound(s) to remain in contact with the target tissue for longer periods of time. A suitable dosing regime for intranasal compositions would be for the patient to inhale slowly through the nose subsequent to the nasal cavity being cleared. During inhalation the composition would be administered to one nostril while the other is manually compressed. This procedure would then be repeated for the other nostril. Typically, one or two sprays per nostril would be administered by the above procedure up to two or three times each day, ideally once daily. Of particular interest are intranasal compositions suitable for once daily administration.

Intranasal compositions may optionally contain one or more suspending agents, one or more preservatives, one or more wetting agents and/or one or more isotonicity adjusting agents as desired. Compositions suitable for intranasal administration may optionally further contain other excipients, such as antioxidants (for example sodium metabisulphite), taste-masking agents (such as menthol) and sweetening agents (for example dextrose, glycerol, saccharin and/or sorbitol).

The suspending agent, if included, will typically be present in the intranasal composition in an amount of between about 0.1 and 5% (w/w), such as between about 1.5% and 2.4% (w/w), based on the total weight of the composition. Examples of suspending agents include Avicel®, carboxymethylcellulose, veegum, tragacanth, bentonite, methylcellulose and polyethylene glycols, e.g. microcrystalline cellulose or carboxy methylcellulose sodium. Suspending agents may also be included in compositions suitable for inhaled, ocular and oral administration as appropriate.

For stability purposes, intranasal compositions comprising a compound of formula (I) or a pharmaceutically acceptable salt thereof may be protected from microbial or fungal contamination and growth by inclusion of a preservative. Examples of pharmaceutically acceptable anti-microbial agents or preservatives may include quaternary ammonium compounds (e.g. benzalkonium chloride, benzethonium chloride, cetrimide and cetylpyridinium chloride), mercurial agents (e.g. phenylmercuric nitrate, phenylmercuric acetate and thimerosal), alcoholic agents (e.g. chlorobutanol, phenylethyl alcohol and benzyl alcohol), antibacterial esters (e.g. esters of para-hydroxybenzoic acid), chelating agents such as disodium ethylenediaminetetraacetate (EDTA) and other anti-microbial agents such as chlorhexidine, chlorocresol, sorbic acid and its salts (such as potassium sorbate) and polymyxin. Examples of pharmaceutically acceptable anti-fungal agents or preservatives may include sodium benzoate. The preservative, if included, may be present in an amount of between about 0.001 and 1% (w/w), such as about 0.015% (w/w), based on the total weight of the composition. Preservatives may be included in compositions suitable for other routes of administration as appropriate.

Compositions which contain a suspended medicament may include a pharmaceutically acceptable wetting agent which functions to wet the particles of medicament to facilitate dispersion thereof in the aqueous phase of the composition. Typically, the amount of wetting agent used will not cause foaming of the dispersion during mixing. Examples of wetting agents include fatty alcohols, esters and ethers, such as polyoxyethylene (20) sorbitan monooleate (Polysorbate 80). The wetting agent may be present in intranasal compositions in an amount of between about 0.001 and 0.05% (w/w), for example about 0.025% (w/w), based on the total weight of the composition. Wetting agents may be included in compositions suitable for other routes of administration, e.g. for inhaled and/or ocular administration, as appropriate.

An isotonicity adjusting agent may be included to achieve isotonicity with body fluids e.g. fluids of the nasal cavity, resulting in reduced levels of irritancy. Examples of isotonicity adjusting agents include sodium chloride, dextrose, xylitol and calcium chloride. An isotonicity adjusting agent may be included in intranasal compositions in an amount of between about 0.1 and 10% (w/w), such as about 5.0% (w/w), based on the total weight of the composition. Isotonicity adjusting agents may also be included in compositions suitable for other routes of administration, for example in compositions suitable for inhaled, ocular, oral liquid and parenteral administration, as appropriate.

Further, the intranasal compositions comprising a compound of formula (I) or a pharmaceutically acceptable salt thereof may be buffered by the addition of suitable buffering agents such as sodium citrate, citric acid, phosphates such as disodium phosphate (for example the dodecahydrate, heptahydrate, dihydrate and anhydrous forms) or sodium phosphate and mixtures thereof. Buffering agents may also be included in compositions suitable for other routes of administration as appropriate.

Compositions for administration topically to the nose or lung for example, for the treatment of rhinitis, include pressurised aerosol compositions and aqueous compositions delivered to the nasal cavities by pressurised pump. Compositions which are non-pressurised and adapted to be administered topically to the nasal cavity are of particular interest. Suitable compositions contain water as the diluent or carrier for this purpose. Aqueous compositions for administration to the lung or nose may be provided with conventional excipients such as buffering agents, tonicity modifying agents and the like. Aqueous compositions may also be administered to the nose by nebulisation.

A fluid dispenser may typically be used to deliver a fluid composition to the nasal cavities. The fluid composition may be aqueous or non-aqueous, but typically aqueous. Such a fluid dispenser may have a dispensing nozzle or dispensing orifice through which a metered dose of the fluid composition is dispensed upon the application of a user-applied force to a pump mechanism of the fluid dispenser. Such fluid dispensers are generally provided with a reservoir of multiple metered doses of the fluid composition, the doses being dispensable upon sequential pump actuations. The dispensing nozzle or orifice may be configured for insertion into the nostrils of the user for spray dispensing of the fluid composition into the nasal cavity. A fluid dispenser of the aforementioned type is described and illustrated in WO05/044354 the entire content of which is hereby incorporated herein by reference. The dispenser has a housing which houses a fluid discharge device having a compression pump mounted on a container for containing a fluid composition. The housing has at least one finger-operable side lever which is movable inwardly with respect to the housing to cam the container upwardly in the housing to cause the pump to compress and pump a metered dose of the composition out of a pump stem through a nasal nozzle of the housing. In one embodiment, the fluid dispenser is of the general type illustrated in FIGS. 30-40 of WO05/044354.

In one aspect, there is provided an intranasal composition comprising a compound of formula (I) or a pharmaceutically acceptable salt thereof. In another aspect, such an intranasal composition is benzalkonium chloride-free.

Inhaled administration involves topical administration to the lung, such as by aerosol or dry powder composition.

Aerosol compositions suitable for inhaled administration may comprise a solution or fine suspension of the compound in a pharmaceutically acceptable aqueous or non-aqueous solvent. Aerosol compositions suitable for inhalation can be either a suspension or a solution and generally contain a compound of formula (I) or a pharmaceutically acceptable salt thereof and a suitable propellant such as a fluorocarbon or hydrogen-containing chlorofluorocarbon or mixtures thereof, such as hydrofluoroalkanes, e.g. 1,1,1,2-tetrafluoroethane, 1,1,1,2,3,3,3-heptafluoro-n-propane or a mixture thereof. The aerosol composition may optionally contain additional excipients well known in the art such as surfactants or cosolvents. Examples of surfactants include, but are not limited to oleic acid, lecithin, an oligolactic acid or derivative e.g. as described in WO94/21229 and WO98/34596. An example of a cosolvent includes, but is not limited to ethanol. Aerosol compositions may be presented in single or multidose quantities in sterile form in a sealed container, which may take the form of a cartridge or refill for use with an atomising device or inhaler. Alternatively, the sealed container may be a unitary dispensing device such as a single dose nasal inhaler or an aerosol dispenser fitted with a metering valve (metered dose inhaler), which is intended for disposal once the contents of the container have been exhausted.

Dry powder inhalable compositions may take the form of capsules and cartridges of, for example, gelatine, or blisters of, for example, laminated aluminium foil, for use in an inhaler or insufflator. Such compositions may be formulated comprising a powder mix of a compound of formula (I) or a pharmaceutically acceptable salt thereof and a suitable powder base such as lactose or starch.

Optionally, for dry powder inhalable compositions, a composition suitable for inhaled administration may be incorporated into a plurality of sealed dose containers (e.g. comprising the dry powder composition) mounted longitudinally in a strip or ribbon inside a suitable inhalation device. The container is rupturable or peel-openable on demand and the dose of e.g. the dry powder composition may be administered by inhalation via the device such as the DISKUS™ device, marketed by GlaxoSmithKline. The DISKUS™ inhalation device is for example described in GB 2242134 A, and in such a device, at least one container for the composition in powder form (the container or containers may, for example, be a plurality of sealed dose containers mounted longitudinally in a strip or ribbon) is defined between two members peelably secured to one another; the device comprises: a means of defining an opening station for the said container or containers; a means for peeling the members apart at the opening station to open the container; and an outlet, communicating with the opened container, through which a user can inhale the composition in powder form from the opened container.

Aerosol compositions are typically arranged so that each metered dose or “puff” of aerosol contains about 20 μg-2000 μg, particularly about 20 μg-500 μg of a compound of formula (I) or a pharmaceutically acceptable salt thereof. Administration may be once daily or several times daily, for example 2, 3, 4 or 8 times, giving for example 1, 2 or 3 doses each time. The overall daily dose with an aerosol will be within the range of about 100 μg-10 mg, such as between about 200 μg-2000 μg. The overall daily dose and the metered dose delivered by capsules and cartridges in an inhaler or insufflator will generally be double those with aerosol compositions.

In another aspect, there is provided a pharmaceutical composition comprising a compound of formula (I) or a pharmaceutically acceptable salt thereof which is suitable for epicutaneous administration. An epicutaneous composition to be applied to the affected area e.g. the skin, by one or more application per day, may be in the form of, for example, an ointment, a cream, an emulsion, a lotion, a foam, a spray, an aqueous gel, or a microemulsion. Such compositions may optionally contain one or more solubilising agents, skin-penetration-enhancing agents, surfactants, fragrances, preservatives or emulsifying agents.

Ointments, creams and gels, may, for example, be formulated with an aqueous or oily base with the addition of suitable thickening and/or gelling agent and/or solvents. Such bases may thus, for example, include water and/or an oil such as liquid paraffin or a vegetable oil such as arachis oil or castor oil, or a solvent such as polyethylene glycol. Thickening agents and gelling agents which may be used according to the nature of the base include soft paraffin, aluminium stearate, cetostearyl alcohol, polyethylene glycols, woolfat, beeswax, carboxypolymethylene and cellulose derivatives, and/or glyceryl monostearate and/or non-ionic emulsifying agents. Lotions may be formulated with an aqueous or oily base and will in general also contain one or more emulsifying agents, stabilising agents, dispersing agents, suspending agents or thickening agents.

In another aspect, there is provided a pharmaceutical composition comprising a compound of formula (I) or a pharmaceutically acceptable salt thereof which is suitable for ocular administration. Such compositions may optionally contain one or more suspending agents, one or more preservatives, one or more wetting/lubricating agents and/or one or more isotonicity adjusting agents. Examples of ophthalmic wetting/lubricating agents may include cellulose derivatives, dextran 70, gelatin, liquid polyols, polyvinyl alcohol and povidone such as cellulose derivatives and polyols.

In another aspect, there is provided a pharmaceutical composition comprising a compound of formula (I) or a pharmaceutically acceptable salt thereof which is suitable for oral administration. Tablets and capsules for oral administration may be in unit dose form, and may contain conventional excipients, such as binding agents, fillers, tabletting lubricants, disintegrants and acceptable wetting agents. The tablets may be coated according to methods well known in normal pharmaceutical practice.

Oral liquid preparations may be in the form of, for example, aqueous or oily suspension, solutions, emulsions, syrups or elixirs, or may be in the form of a dry product for reconstitution with water or other suitable vehicle before use. Such liquid preparations may contain conventional additives such as suspending agents, emulsifying agents, non-aqueous vehicles (which may include edible oils), preservatives, and, if desired, conventional flavourings or colorants.

In another aspect, there is provided a pharmaceutical composition comprising a compound of formula (I) or a pharmaceutically acceptable salt thereof which is suitable for parenteral administration. Fluid unit dosage forms suitable for parenteral administration may be prepared utilising a compound of formula (I) or pharmaceutically acceptable salt thereof and a sterile vehicle which may be aqueous or oil based. The compound, depending on the vehicle and concentration used, may be either suspended or dissolved in the vehicle. In preparing solutions, the compound may be dissolved for injection and filter sterilised before filling into a suitable vial or ampoule and sealing. Optionally, adjuvants such as a local anaesthetic, preservatives and buffering agents may be dissolved in the vehicle. To enhance the stability, the composition may be frozen after filling into the vial and the water removed under vacuum. The lyophilised parenteral composition may be reconstituted with a suitable solvent just prior to administration. Parenteral suspensions may be prepared in substantially the same manner, except that the compound is suspended in the vehicle instead of being dissolved, and sterilisation cannot be accomplished by filtration. The compound may be sterilised by exposure to ethylene oxide before suspension in a sterile vehicle. A surfactant or wetting agent may be included in the composition to facilitate uniform distribution of the compound.

The compounds and pharmaceutical compositions according to the invention may be used in combination with or include one or more other therapeutic agents, for example selected from anti-inflammatory agents, anticholinergic agents (particularly an M₁/M₂/M₃ receptor antagonist), β₂-adrenoreceptor agonists, antiinfective agents such as antibiotics or antivirals, or antihistamines. The invention thus provides, in a further aspect, a combination comprising a compound of formula (I) or a pharmaceutically acceptable salt thereof together with one or more other therapeutically active agents, for example selected from an anti-inflammatory agent such as a corticosteroid or an NSAID, an anticholinergic agent, a β₂-adrenoreceptor agonist, an antiinfective agent such as an antibiotic or an antiviral, or an antihistamine. One embodiment of the invention encompasses combinations comprising a compound of formula (I) or a pharmaceutically acceptable salt thereof together with a β₂-adrenoreceptor agonist, and/or an anticholinergic, and/or a PDE-4 inhibitor, and/or an antihistamine.

One embodiment of the invention encompasses combinations comprising one or two other therapeutic agents.

It will be clear to a person skilled in the art that, where appropriate, the other therapeutic ingredient(s) may be used in the form of salts, for example as alkali metal or amine salts or as acid addition salts, or prodrugs, or as esters, for example lower alkyl esters, or as solvates, for example hydrates to optimise the activity and/or stability and/or physical characteristics, such as solubility, of the therapeutic ingredient. It will be clear also that, where appropriate, the therapeutic ingredients may be used in optically pure form.

The invention thus provides, in another aspect, a combination comprising a compound of formula (I) or a pharmaceutically acceptable salt thereof together with a β₂-adrenoreceptor agonist

Examples of β₂-adrenoreceptor agonists include salmeterol (which may be a racemate or a single enantiomer such as the R-enantiomer), salbutamol (which may be a racemate or a single enantiomer such as the R-enantiomer), formoterol (which may be a racemate or a single diastereomer such as the R,R-diastereomer), salmefamol, fenoterol, carmoterol, etanterol, naminterol, clenbuterol, pirbuterol, flerbuterol, reproterol, bambuterol, indacaterol, terbutaline and salts thereof, for example the xinafoate (1-hydroxy-2-naphthalenecarboxylate) salt of salmeterol, the sulphate salt or free base of salbutamol or the fumarate salt of formoterol. In one embodiment the β₂-adrenoreceptor agonists are long-acting β₂-adrenoreceptor agonists, for example, compounds which provide effective bronchodilation for about 12 hours or longer. A further example of a β₂-adrenoreceptor agonist is the compound 4-{(1R)-2-[(6-{2-[(2,6-dichlorophenyl)methyoxy]ethoxy}hexyl)amino]-1-hydroxyethyl}-2-(hydroxyethyl)phenol triphenylacetate (Vilanterol Trifenatate).

Other β₂-adrenoreceptor agonists include those disclosed in WO02/066422, WO02/070490, WO02/076933, WO03/024439, WO03/072539, WO03/091204, WO04/016578, WO04/022547, WO04/037807, WO04/037773, WO04/037768, WO04/039762, WO04/039766, WO01/42193 and WO03/042160.

Examples of β₂-adrenoreceptor agonists include:
3-(4-{[6-({(2R)-2-hydroxy-2-[4-hydroxy-3-(hydroxymethyl)phenyl]ethyl}amino) hexyl]oxy}butyl)benzenesulfonamide;
3-(3-{[7-({(2R)-2-hydroxy-2-[4-hydroxy-3-hydroxymethyl)phenyl]ethyl}-amino) heptyl]oxy}propyl)benzenesulfonamide;
4-{(1R)-2-[(6-{2-[(2,6-dichlorobenzyl) oxy]ethoxy}hexyl)amino]-1-hydroxyethyl}-2-(hydroxymethyl)phenol;
4-{(1R)-2-[(6-{4-[3-(cyclopentylsulfonyl)phenyl]butoxy}hexyl)amino]-1-hydroxyethyl}-2-(hydroxymethyl)phenol;
N-[2-hydroxyl-5-[(1R)-1-hydroxy-2-[[2-4-[[(2R)-2-hydroxy-2-phenylethyl]amino]phenyl]ethyl]amino]ethyl]phenyl]formamide;
N-2{2-[4-(3-phenyl-4-methoxyphenyl)aminophenyl]ethyl}-2-hydroxy-2-(8-hydroxy-2(1H)-quinolinon-5-yl)ethylamine; and
5-[(R)-2-(2-{4-[4-(2-amino-2-methyl-propoxy)-phenylamino]-phenyl}-ethylamino)-1-hydroxy-ethyl]-8-hydroxy-1H-quinolin-2-one.

The β₂-adrenoreceptor agonist may be in the form of a salt formed with a pharmaceutically acceptable acid selected from sulphuric, hydrochloric, fumaric, hydroxynaphthoic (for example 1- or 3-hydroxy-2-naphthoic), cinnamic, substituted cinnamic, triphenylacetic, sulphamic, sulphanilic, naphthaleneacrylic, benzoic, 4-methoxybenzoic, 2- or 4-hydroxybenzoic, 4-chlorobenzoic and 4-phenylbenzoic acid.

The invention thus provides, in another aspect, a combination comprising a compound of formula (I) or a pharmaceutically acceptable salt thereof together with a corticosteroid.

Suitable anti-inflammatory agents include corticosteroids. Examples of corticosteroids which may be used in combination with the compounds of the invention are those oral and inhaled corticosteroids and their pro-drugs which have anti-inflammatory activity. Examples include methyl prednisolone, prednisolone, dexamethasone, fluticasone propionate, 6α,9α-difluoro-11β-hydroxy-16α-methyl-17α-[(4-methyl-1,3-thiazole-5-carbonyl)oxy]-3-oxo-androsta-1,4-diene-17β-carbothioic acid S-fluoromethyl ester, 6α,9α-difluoro-17α-[(2-furanylcarbonyl)oxy]-11β-hydroxy-16α-methyl-3-oxo-androsta-1,4-diene-17β-carbothioic acid S-fluoromethyl ester (fluticasone furoate), 6α,9α-difluoro-11β-hydroxy-16α-methyl-3-oxo-17α-propionyloxy-androsta-1,4-diene-17β-carbothioic acid S-(2-oxo-tetrahydro-furan-3S-yl) ester, 6α,9α-difluoro-11β-hydroxy-16α-methyl-3-oxo-17α-(2,2,3,3-tetramethycyclopropylcarbonyl)oxy-androsta-1,4-diene-17β-carbothioic acid S-cyanomethyl ester and 6α,9α-difluoro-11β-hydroxy-16α-methyl-17α-(1-methycyclopropylcarbonyl)oxy-3-oxo-androsta-1,4-diene-17β-carbothioic acid S-fluoromethyl ester, beclomethasone esters (for example the 17-propionate ester or the 17,21-dipropionate ester), budesonide, flunisolide, mometasone esters (for example mometasone furoate), triamcinolone acetonide, rofleponide, ciclesonide (16α,17-[[(R)-cyclohexylmethylene]bis(oxy)]-11β,21-dihydroxy-pregna-1,4-diene-3,20-dione), butixocort propionate, RPR-106541, and ST-126. In one embodiment corticosteroids include fluticasone propionate, 6α,9α-difluoro-11β-hydroxy-16α-methyl-17α-[(4-methyl-1,3-thiazole-5-carbonyl)oxy]-3-oxo-androsta-1,4-diene-17β-carbothioic acid S-fluoromethyl ester, 6α,9α-difluoro-17α-[(2-furanylcarbonyl)oxy]-11β-hydroxy-16α-methyl-3-oxo-androsta-1,4-diene-17β-carbothioic acid S-fluoromethyl ester, 6α,9α-difluoro-11β-hydroxy-16α-methyl-3-oxo-17α-(2,2,3,3-tetramethycyclopropylcarbonyl)oxy-androsta-1,4-diene-17β-carbothioic acid S-cyanomethyl ester and 6α,9α-difluoro-11β-hydroxy-16α-methyl-17α-(1-methycyclopropylcarbonyl)oxy-3-oxo-androsta-1,4-diene-17β-carbothioic acid S-fluoromethyl ester. In one embodiment the corticosteroid is 6α,9α-difluoro-17α-[(2-furanylcarbonyl)oxy]-11β-hydroxy-16α-methyl-3-oxo-androsta-1,4-d iene-17β-carbothioic acid S-fluoromethyl ester.

Further examples of corticosteroids may include those disclosed in WO02/088167, WO02/100879, WO02/12265, WO02/12266, WO05/005451, WO05/005452, WO06/072599 and WO06/072600.

Non-steroidal compounds having glucocorticoid agonism that may possess selectivity for transrepression over transactivation and that may be useful in combination therapy include those covered in the following published patent applications and patents: WO03/082827, WO98/54159, WO04/005229, WO04/009017, WO04/018429, WO03/104195, WO03/082787, WO03/082280, WO03/059899, WO03/101932, WO02/02565, WO01/16128, WO00/66590, WO03/086294, WO04/026248, WO03/061651, WO03/08277, WO06/000401, WO06/000398, WO06/015870, WO06/108699, WO07/000,334 and WO07/054,294.

Examples of anti-inflammatory agents include non-steroidal anti-inflammatory drugs (NSAID's).

Examples of NSAID's include sodium cromoglycate, nedocromil sodium, phosphodiesterase (PDE) inhibitors (for example, theophylline, PDE4 inhibitors or mixed PDE3/PDE4 inhibitors), leukotriene antagonists, inhibitors of leukotriene synthesis (for example montelukast), iNOS inhibitors, tryptase and elastase inhibitors, beta-2 integrin antagonists and adenosine receptor agonists or antagonists (e.g. adenosine 2a agonists), cytokine antagonists (for example chemokine antagonists, such as a CCR3 antagonist) or inhibitors of cytokine synthesis, or 5-lipoxygenase inhibitors. An iNOS (inducible nitric oxide synthase inhibitor) is preferably for oral administration. Examples of iNOS inhibitors include those disclosed in WO93/13055, WO98/30537, WO02/50021, WO95/34534 and WO99/62875. Examples of CCR3 inhibitors include those disclosed in WO02/26722.

The invention thus provides, in another aspect, a combination comprising a compound of formula (I) or a pharmaceutically acceptable salt thereof together with a PDE4 inhibitor.

In one embodiment the invention provides the use of the compounds of formula (I) or a pharmaceutically acceptable salt thereof in combination with a phosphodiesterase 4 (PDE4) inhibitor, especially in the case of a formulation adapted for inhalation. The PDE4-specific inhibitor useful in this aspect of the invention may be any compound that is known to inhibit the PDE4 enzyme or which is discovered to act as a PDE4 inhibitor, and which are only PDE4 inhibitors, not compounds which inhibit other members of the PDE family, such as PDE3 and PDE5, as well as PDE4.

Compounds include cis-4-cyano-4-(3-cyclopentyloxy-4-methoxyphenyl)cyclohexan-1-carboxylic acid, 2-carbomethoxy-4-cyano-4-(3-cyclopropylmethoxy-4-difluoromethoxyphenyl)cyclohexan-1-one and cis-[4-cyano-4-(3-cyclopropylmethoxy-4-difluoromethoxyphenyl)cyclohexan-1-ol]. Also, cis-4-cyano-4-[3-(cyclopentyloxy)-4-methoxyphenyl]cyclohexane-1-carboxylic acid (also known as cilomilast) and its salts, esters, pro-drugs or physical forms, which is described in U.S. Pat. No. 5,552,438 issued 3 Sep., 1996; this patent and the compounds it discloses are incorporated herein in full by reference.

Other compounds include AWD-12-281 from Elbion (Hofgen, N. et al. 15th EFMC Int Symp Med Chem (September 6-10, Edinburgh) 1998, Abst P. 98; CAS reference No. 247584020-9); a 9-benzyladenine derivative nominated NCS-613 (INSERM); D-4418 from Chiroscience and Schering-Plough; a benzodiazepine PDE4 inhibitor identified as CI-1018 (PD-168787) and attributed to Pfizer; a benzodioxole derivative disclosed by Kyowa Hakko in WO99/16766; K-34 from Kyowa Hakko; V-11294A from Napp (Landells, L. J. et al. Eur Resp J [Annu Cong Eur Resp Soc (September 19-23, Geneva) 1998] 1998, 12 (Suppl. 28): Abst P2393); roflumilast (CAS reference No 162401-32-3) and a pthalazinone (WO99/47505, the disclosure of which is hereby incorporated by reference) from Byk-Gulden; Pumafentrine, (−)-p-[(4aR*,10bS*)-9-ethoxy-1,2,3,4,4a,10b-hexahydro-8-methoxy-2-methylbenzo[c][1,6]naphthyridin-6-yl]-N,-diisopropylbenzamide which is a mixed PDE3/PDE4 inhibitor which has been prepared and published on by Byk-Gulden, now Altana; arofylline under development by Almirall-Prodesfarma; VM554/UM565 from Vernalis; or T-440 (Tanabe Seiyaku; Fuji, K. et al. J Pharmacol Exp Ther, 1998, 284(1): 162), and T2585.

Further compounds are disclosed in the published international patent application WO04/024728 (Glaxo Group Ltd), WO04/056823 (Glaxo Group Ltd) and WO04/103998 (Glaxo Group Ltd).

The invention thus provides, in another aspect, a combination comprising a compound of formula (I) or a pharmaceutically acceptable salt thereof together with an anticholinergic.

Examples of anticholinergic agents are those compounds that act as antagonists at the muscarinic receptors, in particular those compounds which are antagonists of the M₁ or M₃ receptors, dual antagonists of the M₁/M₃ or M₂/M₃, receptors or pan-antagonists of the M₁/M₂/M₃ receptors. Exemplary compounds for administration via inhalation include ipratropium (for example, as the bromide, CAS 22254-24-6, sold under the name Atrovent), oxitropium (for example, as the bromide, CAS 30286-75-0) and tiotropium (for example, as the bromide, CAS 136310-93-5, sold under the name Spiriva). Also of interest are revatropate (for example, as the hydrobromide, CAS 262586-79-8) and LAS-34273 which is disclosed in WO01/04118. Exemplary compounds for oral administration include pirenzepine (CAS 28797-61-7), darifenacin (CAS 133099-04-4, or CAS 133099-07-7 for the hydrobromide sold under the name Enablex), oxybutynin (CAS 5633-20-5, sold under the name Ditropan), terodiline (CAS 15793-40-5), tolterodine (CAS 124937-51-5, or CAS 124937-52-6 for the tartrate, sold under the name Detrol), otilonium (for example, as the bromide, CAS 26095-59-0, sold under the name Spasmomen), trospium chloride (CAS 10405-02-4) and solifenacin (CAS 242478-37-1, or CAS 242478-38-2 for the succinate also known as YM-905 and sold under the name Vesicare).

Other anticholinergic agents include compounds which are disclosed in U.S. patent application 60/487,981 including, for example:
(3-endo)-3-(2,2-di-2-thienylethenyl)-8,8-dimethyl-8-azoniabicyclo[3.2.1]octane bromide;
(3-endo)-3-(2,2-diphenylethenyl)-8,8-dimethyl-8-azoniabicyclo[3.2.1]octane bromide;
(3-endo)-3-(2,2-diphenylethenyl)-8,8-dimethyl-8-azoniabicyclo[3.2.1]octane 4-methylbenzenesulfonate;
(3-endo)-8,8-dimethyl-3-[2-phenyl-2-(2-thienyl)ethenyl]-8-azoniabicyclo[3.2.1]octane bromide; and/or
(3-endo)-8,8-dimethyl-3-[2-phenyl-2-(2-pyridinyl)ethenyl]-8-azoniabicyclo[3.2.1]octane bromide.

Further anticholinergic agents include compounds which are disclosed in U.S. patent application 60/511,009 including, for example:
(endo)-3-(2-methoxy-2,2-di-thiophen-2-yl-ethyl)-8,8-dimethyl-8-azonia-bicyclo[3.2.1]octane iodide;
3-((endo)-8-methyl-8-aza-bicyclo[3.2.1]oct-3-yl)-2,2-diphenyl-propionitrile;
(endo)-8-methyl-3-(2,2,2-triphenyl-ethyl)-8-aza-bicyclo[3.2.1]octane;
3-((endo)-8-methyl-8-aza-bicyclo[3.2.1]oct-3-yl)-2,2-diphenyl-propionamide;
3-((endo)-8-methyl-8-aza-bicyclo[3.2.1]oct-3-yl)-2,2-diphenyl-propionic acid;
(endo)-3-(2-cyano-2,2-diphenyl-ethyl)-8,8-dimethyl-8-azonia-bicyclo[3.2.1]octane iodide;
(endo)-3-(2-cyano-2,2-diphenyl-ethyl)-8,8-dimethyl-8-azonia-bicyclo[3.2.1]octane bromide;
3-((endo)-8-methyl-8-aza-bicyclo[3.2.1]oct-3-yl)-2,2-diphenyl-propan-1-ol;
N-benzyl-3-((endo)-8-methyl-8-aza-bicyclo[3.2.1]oct-3-yl)-2,2-diphenyl-propionamide;
(endo)-3-(2-carbamoyl-2,2-diphenyl-ethyl)-8,8-dimethyl-8-azonia-bicyclo[3.2.1]octane iodide;
1-benzyl-[3-[3-((endo)-8-methyl-8-aza-bicyclo[3.2.1]oct-3-yl)-2,2-diphenyl-propyl]-urea;
1-ethyl-3-[3-((endo)-8-methyl-8-aza-bicyclo[3.2.1]oct-3-yl)-2,2-diphenyl-propyl]-urea;
N-[3-((endo)-8-methyl-8-aza-bicyclo[3.2.1]oct-3-yl)-2,2-diphenyl-propyl]-acetamide;
N-[3-((endo)-8-methyl-8-aza-bicyclo[3.2.1]oct-3-yl)-2,2-diphenyl-propyl]-benzamide;
3-((endo)-8-methyl-8-aza-bicyclo[3.2.1]oct-3-yl)-2,2-di-thiophen-2-yl-propionitrile;
(endo)-3-(2-cyano-2,2-di-thiophen-2-yl-ethyl)-8,8-dimethyl-8-azonia-bicyclo[3.2.1]octane iodide;
N-[3-((endo)-8-methyl-8-aza-bicyclo[3.2.1]oct-3-yl)-2,2-diphenyl-propyl]-benzenesulfonamide;
[3-((endo)-8-methyl-8-aza-bicyclo[3.2.1]oct-3-yl)-2,2-diphenyl-propyl]-urea;
N-[3-((endo)-8-methyl-8-aza-bicyclo[3.2.1]oct-3-yl)-2,2-diphenyl-propyl]-methanesulfonamide; and/or
(endo)-3-{2,2-diphenyl-3-[(1-phenyl-methanoyl)-amino]-propyl}-8,8-dimethyl-8-azonia-bicyclo[3.2.1]octane bromide.

Further compounds include:
(endo)-3-(2-methoxy-2,2-di-thiophen-2-yl-ethyl)-8,8-dimethyl-8-azonia-bicyclo[3.2.1]octane iodide;
(endo)-3-(2-cyano-2,2-diphenyl-ethyl)-8,8-dimethyl-8-azonia-bicyclo[3.2.1]octane iodide;
(endo)-3-(2-cyano-2,2-diphenyl-ethyl)-8,8-dimethyl-8-azonia-bicyclo[3.2.1]octane bromide;
(endo)-3-(2-carbamoyl-2,2-diphenyl-ethyl)-8,8-dimethyl-8-azonia-bicyclo[3.2.1]octane iodide;
(endo)-3-(2-cyano-2,2-di-thiophen-2-yl-ethyl)-8,8-dimethyl-8-azonia-bicyclo[3.2.1]octane iodide; and/or
(endo)-3-{2,2-diphenyl-3-[(1-phenyl-methanoyl)-amino]-propyl}-8,8-dimethyl-8-azonia-bicyclo[3.2.1]octane bromide.

The invention thus provides, in another aspect, a combination comprising a compound of formula (I) or a pharmaceutically acceptable salt thereof together with an antihistamine.

In one embodiment the invention provides a combination comprising a compound of formula (I), or a pharmaceutically acceptable salt thereof, together with an H1 antagonist. Examples of H1 antagonists include, without limitation, amelexanox, astemizole, azatadine, azelastine, acrivastine, brompheniramine, cetirizine, levocetirizine, efletirizine, chlorpheniramine, clemastine, cyclizine, carebastine, cyproheptadine, carbinoxamine, descarboethoxyloratadine, doxylamine, dimethindene, ebastine, epinastine, efletirizine, fexofenadine, hydroxyzine, ketotifen, loratadine, levocabastine, mizolastine, mequitazine, mianserin, noberastine, meclizine, norastemizole, olopatadine, picumast, pyrilamine, promethazine, terfenadine, tripelennamine, temelastine, trimeprazine and triprolidine, particularly cetirizine, levocetirizine, efletirizine and fexofenadine. In a further embodiment the invention provides a combination comprising a compound of formula (I), or a pharmaceutically acceptable salt thereof, together with an H3 antagonist (and/or inverse agonist). Examples of H3 antagonists include, for example, those compounds disclosed in WO2004/035556 and in WO2006/045416. Other histamine receptor antagonists which may be used in combination with the compounds of formula (I), or a pharmaceutically acceptable salt thereof, include antagonists (and/or inverse agonists) of the H4 receptor, for example, the compounds disclosed in Jablonowski et al., J. Med. Chem. 46:3957-3960 (2003).

The invention thus provides, in another aspect, a combination comprising a compound of formula (I) or a pharmaceutically acceptable salt thereof together with another calcium release activated calcium channel inhibitor.

The invention thus provides, in a further aspect, a combination comprising a compound of formula (I) or a pharmaceutically acceptable salt thereof together with an anticholinergic and a PDE-4 inhibitor.

The individual compounds of such combinations may be administered either sequentially or simultaneously in separate or combined pharmaceutical formulations. In one embodiment, the individual compounds will be administered simultaneously in a combined pharmaceutical composition. Appropriate doses of known therapeutic agents will readily be appreciated by those skilled in the art.

The combinations referred to above may conveniently be presented for use in the form of a pharmaceutical composition and thus pharmaceutical compositions comprising a combination as defined above together with a pharmaceutically acceptable diluent or carrier represent a further aspect of the invention.

The individual compounds of such combinations may be administered either sequentially in separate pharmaceutical compositions as well as simultaneously in combined pharmaceutical compositions. Additional therapeutically active ingredients may be suspended in the composition together with a compound of formula (I). Appropriate doses of known therapeutic agents will be readily appreciated by those skilled in the art.

The compounds of the invention may be prepared by the methods described below or by similar methods. Thus the following Intermediates and Examples serve to illustrate the preparation of the compounds of the invention, and are not to be considered as limiting the scope of the invention in any way.

### EXAMPLES

### General

Mass directed autopreparative HPLC was undertaken under the conditions given below. The UV detection was an averaged signal from wavelength of 210 nm to 350 nm and mass spectra were recorded on a mass spectrometer using alternate-scan positive and negative mode electrospray ionization.

### Method A:

Method A was conducted on a Supelcosil ABZ+Plus column (typically 150 mm×30 mm i.d. 5 μm packing diameter) at ambient temperature. The solvents employed were:

A=0.1% v/v solution of formic acid in water
B=MeCN:Water 95:5+0.05% formic acid

### Method B:

Method B was conducted on a Sunfire C₁₈ column (typically 150 mm×30 mm i.d. 5 μm packing diameter) at ambient temperature. The solvents employed were:

A=0.1% v/v solution of Formic acid in water
B=0.1% v/v solution of Formic acid in acetonitrile.

### Method C:

Method C was conducted on a Sunfire C₁₈ column (typically 150 mm×30 mm i.d. 5 μm packing diameter) at ambient temperature. The solvents employed were:

A=0.1% v/v solution of trifluoroacetic acid in water
B=0.1% v/v solution of trifluoroacetic acid in acetonitrile.

### Method D:

Method D was conducted on a Sunfire C₁₈ column (typically 100 mm×19 mm i.d. 5 μm packing diameter) at ambient temperature. The solvents employed were:

A=0.1% v/v solution of trifluoroacetic acid in water
B=0.1% v/v solution of trifluoroacetic acid in acetonitrile.

### Method E:

Method E was conducted on an XBridge C₁₈ column (typically 150 mm×19 mm i.d. 5 μm packing diameter) at ambient temperature. The solvents employed were:

A=10 mM aqueous ammonium bicarbonate adjusted to pH 10 with ammonia solution.
B=acetonitrile.

### Experimental Details

Experimental details of LC-MS systems 1-8 as referred to herein are as follows:

### System 1

Column: 3.3 cm×4.6 mm ID, 3 μm ABZ+PLUS from Supelco®
Flow Rate: 3 mL/min.
Temp.: Room temperature
UV detection range: 215 to 330 nm
Mass spectrum: Recorded on a mass spectrometer using alternative-scan positive and negative mode electrospray ionisation.
Solvents: A: 0.1% formic acid+10 mM ammonium acetate
   B: 95% acetonitrile+0.05% formic acid

| | Time (min.) | A % | B % |
|---|---|---|---|
| Gradient: | 0 | 100 | 0 |
| | 0.7 | 100 | 0 |
| | 4.2 | 0 | 100 |
| | 4.6 | 0 | 100 |
| | 4.8 | 100 | 0 |

### System 2

Column: 50 mm×2.1 mm ID, 1.7 μm Acquity HPLC BEH C₁₈

Flow Rate: 1 mL/min.

### Temp.: 40° C.

### UV Detection Range: 220 to 330 nm

Mass spectrum: Recorded on a mass spectrometer using alternative-scan positive and negative mode electrospray ionisation.
Solvents: A: 0.1% formic acid+10 mM ammonium acetate
   B: 95% acetonitrile+0.05% formic acid

| | Time (min.) | A % | B % |
|---|---|---|---|
| Gradient: | 0 | 97 | 3 |
| | 0.1 | 97 | 3 |
| | 1.4 | 0 | 100 |
| | 1.9 | 0 | 100 |
| | 2 | 97 | 3 |

### System 3

Column: 50 mm×2.1 mm ID, 1.7 μm Acquity HPLC BEH C₁₈
Flow Rate: 1 mL/min.

### Temp.: 40° C.

### UV Detection Range: 210 to 350 nm

Mass spectrum: Recorded on a mass spectrometer using alternative-scan positive and negative mode electrospray ionisation.
Solvents: A: 0.1% v/v formic acid in water
   B: 0.1% v/v formic acid in acetonitrile

| | Time (min.) | A % | B % |
|---|---|---|---|
| Gradient: | 0 | 97 | 3 |
| | 1.5 | 0 | 100 |
| | 1.9 | 0 | 100 |
| | 2.0 | 97 | 3 |

### System 4

Column: 30 mm×4.6 mm ID, 3.5 μm Sunfire C₁₈ column
Flow Rate: 3 mL/min.

### Temp.: 30° C.

### UV Detection Range: 210 to 350 nm

Mass spectrum: Recorded on a mass spectrometer using alternative-scan positive and negative mode electrospray ionisation.
Solvents: A: 0.1% v/v solution of formic acid in water
   B: 0.1% v/v solution of formic acid in acetonitrile

| | Time (min.) | A % | B % |
|---|---|---|---|
| Gradient: | 0 | 97 | 3 |
| | 0.1 | 97 | 3 |
| | 4.2 | 0 | 100 |
| | 4.8 | 0 | 100 |
| | 4.9 | 97 | 3 |
| | 5.0 | 97 | 3 |

### System 5

Column: 50 mm×2.1 mm ID, 1.7 μm Acquity HPLC BEH C₁₈
Flow Rate: 1 mL/min.

### Temp.: 40° C.

### UV Detection Range: 220 to 330 nm

Mass spectrum: Recorded on a mass spectrometer using alternative-scan positive and negative mode electrospray ionisation.
Solvents: A: 0.1% v/v solution of trifluoroacetic acid in water
   B: 0.1% v/v solution of trifluoroacetic acid in acetonitrile

| | Time (min.) | A % | B % |
|---|---|---|---|
| Gradient: | 0 | 97 | 3 |
| | 1.5 | 0 | 100 |
| | 1.9 | 0 | 100 |
| | 2.0 | 97 | 3 |

### System 6

Column: 30 mm×4.6 mm ID, 3.5 μm Sunfire C₁₈ column
Flow Rate: 3 mL/min.

### Temp.: 30° C.

### UV Detection Range: 210 to 350 nm

Mass spectrum: Recorded on a mass spectrometer using alternative-scan positive and negative mode electrospray ionisation.
Solvents: A: 0.1% v/v solution of trifluoroacetic acid in water
   B: 0.1% v/v solution of trifluoroacetic acid in acetonitrile

| | Time (min.) | A % | B % |
|---|---|---|---|
| Gradient: | 0 | 97 | 3 |
| | 0.1 | 97 | 3 |
| | 4.2 | 0 | 100 |
| | 4.8 | 0 | 100 |
| | 4.9 | 97 | 3 |
| | 5.0 | 97 | 3 |

### System 7

Column: 50 mm×2.1 mm ID, 1.7 μm Acquity HPLC BEH C₁₈
Flow Rate: 1 mL/min.

### Temp.: 40° C.

### UV Detection Range: 220 to 350 nm

Mass spectrum: Recorded on a mass spectrometer using alternative-scan positive and negative mode electrospray ionisation.
Solvents: A: 10 mM ammonium bicarbonate in water adjusted to pH10 with ammonia solution
   B: Acetonitrile

| | Time (min.) | A % | B % |
|---|---|---|---|
| Gradient: | 0 | 99 | 1 |
| | 1.5 | 3 | 97 |
| | 1.9 | 3 | 97 |
| | 2.0 | 0 | 100 |

### System 8

Column: 50 mm×4.6 mm ID, 3.5 μm XBridge C₁₈ column
Flow Rate: 3 mL/min.

### Temp.: 30° C.

### UV Detection Range: 210 to 350 nm

Mass spectrum: Recorded on a mass spectrometer using alternative-scan positive and negative mode electrospray ionisation.
Solvents: A: 10 mM ammonium bicarbonate in water adjusted to pH10 with ammonia solution
   B: Acetonitrile

| | Time (min.) | A % | B % |
|---|---|---|---|
| Gradient: | 0 | 99 | 1 |
| | 0.1 | 99 | 1 |
| | 4.0 | 3 | 97 |
| | 5.0 | 3 | 97 |

### ABBREVIATIONS

The following list provides definitions of certain abbreviations as used herein. It will be appreciated that the list is not exhaustive, but the meaning of those abbreviations not herein below defined will be readily apparent to those skilled in the art.
Ac (acetyl);
Bu (butyl);
nBu (n-butyl);
tBu (t-butyl);
DCM (dichloromethane);
DMF (N,N-dimethylformamide);
DMSO (dimethylsulfoxide);
Et (ethyl);
EtOAc (ethyl acetate);
g (grams);
h (hours)
HCl (Hydrochloric acid)
hr (hours);
Hz (Hertz);
L (liters);
LDA (lithium diisopropylamide);
M (molar);
MDAP (mass directed autopreparative HPLC);
Me (methyl);
MeOH (methanol);
mg (milligrams);
MHz (megahertz);
min (minutes);
ml (milliliters);
μl (microliters);
mM (millimolar);
mmol (millimoles);
mol (moles);
Ph (phenyl);
ⁱPr (isopropyl);
Si (Silica)
SPE (solid phase extraction);
TBTU (O-(benzotriazol-1-yl)-N,N,N′,N′-tetramethyluronium tetrafluoroborate);
TEA (triethylamine);
TFA (trifluoroacetic acid);
THF (tetrahydrofuran);
TLC (thin layer chromatography);
TMS (trimethylsilyl);

All references to ether are to diethyl ether and brine refers to a saturated aqueous solution of NaCl.

### Intermediate 1: Methyl 1-({5-chloro-2-[(phenylmethyl)oxy]phenyl}methyl)-1H-pyrazole-3-carboxylate

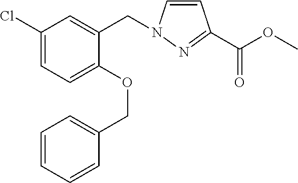

To a solution of 2-(bromomethyl)-4-chloro-1-[(phenylmethyl)oxy]benzene (3.71 g, 11.9 mmol, synthesised according to WO 200606696) in DMF (25 ml) was added 2H-pyrazole-3-carboxylic acid methyl ester (1.5 g, 11.9 mmol, ChemCollect GmbH) and then potassium carbonate (4.18 g, 30.2 mmol). The suspension was stirred at ambient temperature overnight. The suspension was partitioned between water (150 ml) and ethyl acetate (150 ml). The phases were separated and the aqueous phase washed with ethyl acetate (100 ml). The combined organic extracts were washed with brine (2×50 ml), dried (MgSO₄), filtered and the solvent removed in vacuo to leave an oil. The residue was loaded in dichloromethane and purified on silica (Si) 2×100 g using 0-50% ethyl acetate-cyclohexane. The appropriate fractions were combined and evaporated in vacuo to give the title compound as a colourless oil (2.27 g); LCMS: (System 1) MH⁺=357, t_{RET}=3.61 min.

### Intermediate 2: 1-({5-Chloro-2-[(phenyl methyl)oxy]phenyl}methyl)-1H-pyrazole-3-carboxylic acid

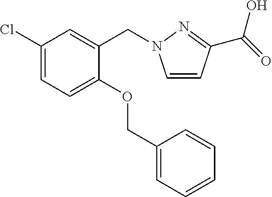

To a solution of methyl 1-({5-chloro-2-[(phenylmethyl)oxy]phenyl}methyl)-1H-pyrazole-3-carboxylate (2.23 g, 6.25 mmol) in ethanol (25 ml) was added 2M aqueous sodium hydroxide (7.5 ml, 15.0 mmol). The solution was heated to 90° C. (reflux) in a drysyn block overnight. The solvent was removed in vacuo and the aqueous residue partitioned between ethyl acetate (180 ml) and water (100 ml). To the biphasic mixture was cautiously added 2 M aqueous HCl (20 ml) and the phases separated. The aqueous phase was extracted with ethyl acetate (70 ml). The combined organic extracts were dried (MgSO₄), filtered and the solvent removed in vacuo to give the title compound as a white solid (2.39 g); LCMS: (System 1) MH⁺=343, t_{RET}=3.30 min.

### Intermediate 3:1-[(5-Chloro-2-hydroxyphenyl)methyl]-N-(2,6-difluorophenyl)-1H-pyrazole-3-carboxamide

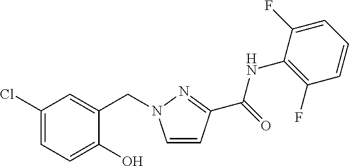

A solution of 1-({5-chloro-2-[(phenylmethyl)oxy]phenyl}methyl)-N-(2,6-difluorophenyl)-1H-pyrazole-3-carboxamide (0.640 g, 1.41 mmol) in ethyl acetate (60 ml) was hydrogenated using an H-cube (settings: ambient, 1 bar, 1 ml/min flow rate) and 5% Pd/C CatCart 30 as the catalyst. The solvent was removed in vacuo to give the title compound as a white solid (0.515 g); LCMS: (System 1) MH⁺=364, t_{RET}=3.17 min.

### Intermediate 4: N-(2,6-difluorophenyl)-1H-pyrazole-3-carboxamide.

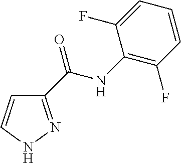

To 1H-pyrazole-3-carboxylic acid (5 g, 44.6 mmol, Manchester Organics Ltd) in N,N-dimethylformamide (60 ml) was added N,N-diisopropylethylamine (15.5 ml, 89 mmol, Apollo Scientific) followed by TBTU (14.32 g, 44.6 mmol) and the mixture stirred at room temperature for 15 min. To this mixture was added 2,6-difluoroaniline (4.8 ml, 44.6 mmol, Aldrich) and the mixture left to stir at 75° C. over the weekend. The reaction mixture was added to water (300 ml) and extracted with EtOAc (3×250 ml). The combined extracts were washed twice with saturated sodium bicarbonate solution, lithium chloride solution and saturated brine solution. The organic layer was dried over magnesium sulphate, the drying agent filtered off and the filtrate evaporated to dryness to leave the crude product. The residue was triturated with DCM, the solid filtered off, washed with DCM and dried in vacuo to leave the title compound as a pinkish solid (5.5 g). A second batch was obtained from the filtrate as above, trituration to leave the title compound as a white solid (275 mg); LCMS: (System 4) MH⁺=224, t_{RET}=1.49 min.

### Intermediate 5: N-(2,6-Difluorophenyl)-1-[(2-hydroxyphenyl)methyl]-1H-pyrazole-3-carboxamide

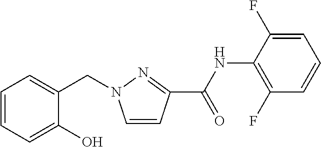

To a solution of N-(2,6-difluorophenyl)-1-({2-[(phenylmethyl)oxy]phenyl}methyl)-1H-pyrazole-3-carboxamide (0.355 g, 0.846 mmol) and ammonium formate (0.352 g, 5.58 mmol, Fluka) in ethanol (10 ml) was added 10% palladium on carbon (0.045 g, 0.423 mmol, Aldrich). The reaction was stirred for 1 h under nitrogen at ambient temperature. The reaction mixture was filtered through a celite SPE cartridge and the solvent removed in vacuo. The residue was partitioned between ethyl acetate (30 ml) and 2M HCl (20 ml). The phases were separated and the organic extract washed with brine (50 ml). The organic phase was evaporated in vacuo to leave the title compound as an off-white solid (0.290 g); LCMS: (System 4) MH⁺=330, t_{RET}=2.43 min

### Intermediate 6: 2:1 Mixture of 2-(bromomethyl)-4-chloro-1-iodobenzene and 1-bromo-2-(bromomethyl)-4-chlorobenzene

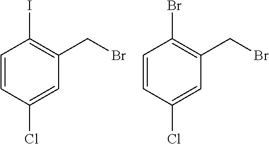

N-bromosuccinimide (6.34 g, 35.6 mmol) was weighed into a flask and carbon tetrachloride (200 ml) was added followed by 4-chloro-1-iodo-2-methylbenzene (8.57 g, 33.9 mmol, Fluorochem Ltd). The reaction mixture was stirred and dibenzoyl peroxide (0.822 g, 3.39 mmol) was added in one portion. The apparatus was then flushed three times with nitrogen and heated to reflux with vigorous stirring. After approximately 46 h at reflux, the reaction was allowed to cool to room temperature. The reaction mixture was filtered to remove the insoluble material and washed with aqueous sodium sulfite solution. The carbon tetrachloride layer was dried over sodium sulfate and evaporated to give a crude product as a pale yellow oil which crystallised on standing. The crude product was triturated with cyclohexane. The white insoluble material (1.30 g) was retained.

The soluble material was then loaded onto 50 g silica SPE (pre-conditioned with cyclohexane) and was eluted with cyclohexane. Two product batches were obtained from this purification. The slower running material was obtained as a clear liquid which crystallised on standing to give the title compounds as a 2:1 mixture of 2-(bromomethyl)-4-chloro-1-iodobenzene and 1-bromo-2-(bromomethyl)-4-chlorobenzene (1.99 g).

The faster running material (2.07 g) was combined with the triturated material (1.30 g) and was re-purified on 50 g silica SPE eluting with cyclohexane, exactly as before. A second batch of the title compounds, again as a 2:1 of 2-(bromomethyl)-4-chloro-1-iodobenzene and 1-bromo-2-(bromomethyl)-4-chlorobenzene (2.38 g) was obtained; LCMS: (System 2) t_{RET}=1.37 min (no ions detected).

### Intermediate 7: 1-[(5-Chloro-2-iodophenyl)methyl]-N-(2,6-difluorophenyl)-1H-pyrazole-3-carboxamide

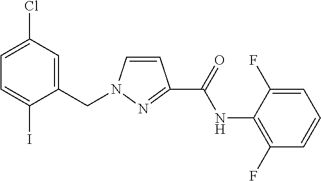

N-(2,6-difluorophenyl)-1H-pyrazole-3-carboxamide (350 mg, 1.57 mmol), 2:1 Mixture of 2-(bromomethyl)-4-chloro-1-iodobenzene and 1-bromo-2-(bromomethyl)-4-chlorobenzene (495 mg, 1.57 mmol) and potassium carbonate (433 mg, 3.14 mmol) were weighed into a flask. DMF (15 ml) was then added and the reaction was stirred at ambient temperature under nitrogen.

After 16 h, the reaction mixture was partitioned between DCM (100 ml) and water (100 ml). The layers were separated and the aqueous layer extracted with further DCM (50 ml). The combined DCM extracts were dried (sodium sulfate) and evaporated to give a crude product as an oil. This product was re-dissolved in DCM and loaded on to 50 g silica SPE which had been pre-equilibrated with cyclohexane. The product was then purified using 0-50% ethyl acetate-cyclohexane gradient to give a white solid (2:1 mixture of iodide and bromide).

200 mg of the product mixture was purified by MDAP (2×100 mg batches). Each sample was dissolved in 1:1 MeOH:DMSO (1 ml) and purified (supelcosil ABZ+Plus column) (Method A) eluting with solvents A/B (A: Water+0.1% Formic acid, B:MeCN:Water 95:5+0.05% Formic acid).

The first eluted product from each run was combined and partitioned between NaHCO₃ (aq) and DCM. The layers were separated and the aqueous layer was extracted with a second portion of DCM. The DCM extract was dried (Na₂SO₄) and evaporated to give 1-[(2-bromo-5-chlorophenyl)methyl]-N-(2,6-difluorophenyl)-1H-pyrazole-3-carboxamide as a colourless oil, 45 mg; LCMS: (System 2) MH⁺=426, 428 and 430 (Cl, Br isotopes), t_{RET}=1.22 min.

The second eluted product from each run was combined and partitioned between NaHCO₃ (aq) and DCM. The layers were separated and the aqueous layer was extracted with a second portion of DCM. The DCM extract was dried (Na₂SO₄) and evaporated to give the title compound as a colourless oil (121 mg); LCMS: (System 2) MH⁺=474 and 476 (Cl isotopes) t_{RET}=1.25 min.

### Intermediate 8: [2-(Butyloxy)phenyl]methanol

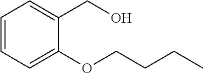

A solution of 2-(hydroxymethyl)phenol (1.24 g, 10 mmol, Aldrich) and 1-bromobutane (1.18 ml, 11 mmol, Acros) in ethanol (5 ml) (total volume ˜7 ml) was mixed in a flow reactor (Vapourtec R4, 30 ml PFA tubing, 110° C.) with a solution of aqueous NaOH (2N, 5 ml, 10 mmol) in water (2 ml). The reagents were each pumped at 0.5 ml/min), giving a reaction time of 30 minutes. After the 30 ml reactor, the solution passed through a second “cooling” reactor (5 ml) at 50° C. At the reactor output, a 250 psi back pressure regulator was used. After all starting solutions had been consumed, the system was purged with an additional 70 ml solvents (1:1, ethanol:water). The collected material was concentrated in vacuo. The residue was partitioned between water and EtOAc, and treated with saturated aqueous sodium bicarbonate solution. The aqueous was further extracted with EtOAc, and the combined organic extracts were dried (hydrophobic frit) and concentrated in vacuo. The residue was purified on silica (100 g) using a 0-100% ethyl acetate-cyclohexane gradient over 40 min. The appropriate fractions were combined and concentrated in vacuo to give the title compound (1.4 g); LCMS: (System 4) t_{RET}=2.51 min, no MH⁺ detected.

### Intermediate 9: 1-(Bromomethyl)-2-(butyloxy)benzene

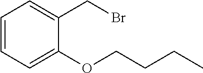

A solution of 2-(butyloxy)phenyl]methanol (6 g, 33.3 mmol) in DCM (30 ml) (total solution volume 34 ml) was mixed in a flow reactor (Vapourtec R4, 35 ml PFA tubing reactor, maintained at 25° C.) with a solution of phosphorus tribromide in DCM (1 molar, 33.6 ml, 33.6 mmol, Aldrich). The solutions were each pumped at 0.58 ml/min), giving a reaction time of 30 min. The output was collected into a stirred flask of water, which was subsequently treated with aqueous sodium bicarbonate (100 ml). The aqueous layer was extracted with DCM (×2), and the combined organic extracts were dried (hydrophobic frit) and concentrated in vacuo to give the title compound as an opaque yellow oil (6.8 g, 84%).

1H NMR (DMSO-d6) 7.38 (1H, dd, J 7.5, 2 Hz), 7.29 (1H, ddd, J 9, 7.5, 2 Hz), 7.00 (1H, d, J=9 Hz), 6.90 (1H, td, J 7.5, 1 Hz), 4.63 (2H, s), 4.03 (2H, t, J=6.5 Hz), 1.78-1.66 (2H, m), 1.56-1.44 (2H, m), 0.94 (3H, t, J=7.5 Hz).

### Intermediate 10: 5-Chloro-2-(hydroxymethyl)phenol

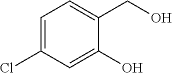

To a solution of 4-chloro-2-hydroxybenzoic acid (2.0 g, 11.6 mmol, Aldrich) in THF (50 ml) was added dropwise 1 M borane in THF (20.9 ml, 20.86 mmol). The suspension was allowed to stir for 30 min at ambient temperature. The mixture was refluxed at 80° C. for 2.5 h. The solvent was removed in vacuo. The residue was loaded in dichloromethane and purified on silica (Si) 100 g using 0-100% ethyl acetate/cyclohexane. The appropriate fractions were combined and evaporated in vacuo to give the title compound as a white solid (1.51 g); LCMS: (System 1) (M-H)⁻=157, t_{RET}=2.44 min.

### Intermediate 11: {4-Chloro-2-[(phenylmethyl)oxy]phenyl}methanol

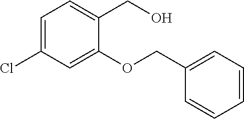

To a solution of 5-chloro-2-(hydroxymethyl)phenol (1.51 g, 9.54 mmol) in ethanol (20 ml), was added sodium hydroxide (5.25 ml, 10.5 mmol). To the mixture was added a solution of benzyl bromide (1.14 ml, 9.54 mmol, Aldrich) in ethanol (30 ml) dropwise. The reaction was stirred under nitrogen overnight at ambient temperature. The solvent was removed in vacuo to leave an aqueous suspension. The suspension was diluted with water (50 ml) and dichloromethane (40 ml). The phases were separated and the aqueous phase extracted with dichloromethane (30 ml). The combined organic extracts were washed with saturated aqueous sodium hydrogen carbonate (20 ml) and the solvent removed in vacuo to leave a white solid (1.52 g). The residue was loaded in dichloromethane and purified on silica (Si) 100 g using 0-50% ethyl acetate-cyclohexane. The appropriate fractions were combined and evaporated in vacuo to give the title compound as a white solid (1.32 g);

LCMS: (System 2) (M-H)⁻=247, t_{RET}=1.17 min.

### Intermediate 12: 1-[(2-Bromophenyl)methyl]-N-(2,6-difluorophenyl)-1H-pyrazole-3-carboxamide

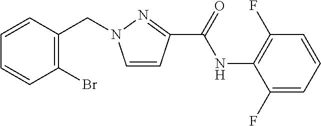

To a mixture of N-(2,6-difluorophenyl)-1H-pyrazole-3-carboxamide (300 mg, 1.34 mmol) and potassium carbonate (374 mg, 2.71 mmol) was added 1-bromo-2-(bromomethyl)benzene (373 mg, 1.49 mmol, Aldrich) in DMF (6 ml). The reaction was stirred overnight, under nitrogen and at ambient temperature. The reaction mixture was diluted with water (80 ml) and ethyl acetate (80 ml). The phases were separated and the organic phase washed with water (80 ml). The organic extract was dried (MgSO₄) and the solvent removed in vacuo. The sample was loaded in dichloromethane and purified on silica (Si) 100 g using 0-50% ethyl acetate-cyclohexane. The appropriate fractions were combined and evaporated in vacuo to give the title compound as a white solid (0.36 g); LCMS: (System 1) MH⁺=394, t_{RET}=3.27 min.

### Intermediate 13: N-(2,6-Difluorophenyl)-1-[(2-iodophenyl)methyl]-1H-pyrazole-3-carboxamide

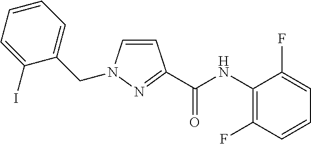

To a mixture of N-(2,6-difluorophenyl)-1H-pyrazole-3-carboxamide (0.5 g, 2.24 mmol), 1-(bromomethyl)-2-iodobenzene (665 mg, 2.24 mmol, ABCR) and potassium carbonate (0.374 g, 2.71 mmol) was added DMF (10 ml). The reaction mixture was stirred overnight, under nitrogen and at ambient temperature. The reaction mixture was diluted with water (80 ml) and ethyl acetate (80 ml). The phases were separated and the organic phase washed with water (80 ml). The organic extract was dried (MgSO₄) and the solvent removed in vacuo. The residue was loaded in dichloromethane and purified on silica (Si) 100 g using 0-50% ethyl acetate-cyclohexane. The appropriate fractions were combined and evaporated in vacuo to give the title compound as a white solid (0.794 g); LCMS: (System 1) MH⁺=440, t_{RET}=3.40 min.

### Intermediate 14: Ethyl 1-{[2-(phenyloxy)phenyl]methyl}-1H-pyrazole-3-carboxlate

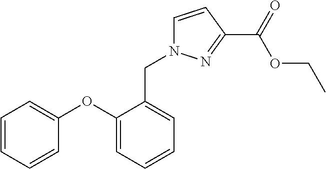

To ethyl 1H-pyrazole-3-carboxylate (1.05 g, 75 mmol, ChemCollect GmbH) in DMF (10 ml) under nitrogen was added sodium hydride (315 mg, 7.88 mmol) and the mixture stirred at room temperature for 10 min. To this mixture was added 1-(bromomethyl)-2-(phenyloxy)benzene (1.97 g, 7.5 mmol, Maybridge) and the mixture left to stir at room temperature for 1 h. The reaction quenched with water and extracted with ethyl acetate (3×). The combined extracts were washed with ˜10% lithium chloride solution in water (3×), the organic layer dried over magnesium sulphate, the drying agent filtered off and the filtrate evaporated to dryness to leave an oil (2.55 g). This was applied to silica SPE (100 g) cartridge, eluting with DCM/EtOAc 0-50% over 60 min. The pure fractions were combined and evaporated to dryness to leave the title compound as dark oil (2.1 g); LCMS: (System 4) MH⁺=323, t_{RET}=3.08 min.

### Intermediate 15: 1-{[2-(Phenyloxy)phenyl]methyl}-1H-pyrazole-3-carboxylic acid

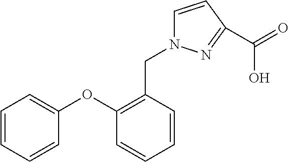

To ethyl 1-{[2-(phenyloxy)phenyl]methyl}-1H-pyrazole-3-carboxylate (6.45 g, 20.0 mmol) in methanol (40 ml) was added 10 N sodium hydroxide (100 ml) and the mixture stirred at reflux for 90 min. The mixture was allowed to cool, evaporated to dryness, the residue suspended in water (600 ml) and the mixture acidified with 2 N HCl solution. The aqueous phase was extracted with ethyl acetate (2×), the combined extracts washed with saturated brine solution and dried over magnesium sulphate. The drying agent was filtered off and the filtrate evaporated to dryness to leave the title compound as a white solid (5.25 g); LCMS: (System 4) MH⁺=295, t_{RET}=2.52 min.

### Intermediate 16: Ethyl 1-{[2-(butyloxy)phenyl]methyl}-1H-pyrazole-3-carboxylate

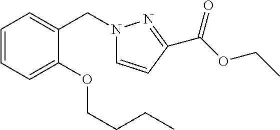

To ethyl 1H-pyrazole-3-carboxylate (1.74 g, 12.4 mmol, ChemCollect GmbH) and potassium carbonate (3.43 g, 24.8 mmol) was added 1-(bromomethyl)-2-(butyloxy)benzene (3.0 g, 12.3 mmol) as a solution in DMF, washing in with further DMF to give a total volume of 35 ml. The mixture was stirred under nitrogen at ambient temperature. After 24 h the reaction was concentrated in vacuo. The residue was partitioned between EtOAc and water (approximately 100 ml each). The layers were separated and the aqueous was extracted with further EtOAc (2×50 ml). The combined organic extracts were washed with brine, dried (MgSO₄) and concentrated in vacuo to give a pale yellow oil, which partially solidified on standing. The material was loaded in dichloromethane and purified on silica (Si) 100 g using 0-100% ethyl acetate-cyclohexane. The appropriate fractions were combined and concentrated in vacuo to give the title compound (2.07 g); LCMS: (System 4) MH⁺=303, t_{RET}=3.27 min.

### Intermediate 17: 1-{[2-(Butyloxy)phenyl]methyl}-1H-pyrazole-3-carboxylic acid

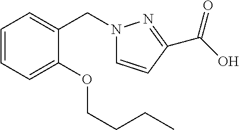

Ethyl 1-{[2-(butyloxy)phenyl]methyl}-1H-pyrazole-3-carboxylate (2.06 g, 6.81 mmol) was dissolved in methanol (10 ml) with stirring. The solution was treated with 2 M aqueous sodium hydroxide (10 ml, 20.0 mmol) and the mixture was heated to reflux (heating block at 100° C.). After 2.5 h the reaction was allowed to cool. The reaction mixture was concentrated in vacuo. The residue was diluted with water (20 ml) and then treated with 2 M HCl added gradually with stirring. This mixture was extracted with diethyl ether (ca. 50 ml), and then DCM (2×50 ml). The organic extracts were washed with brine, combined, dried (MgSO₄) and concentrated to give the title compound as a colourless crystalline solid (1.85 g); LCMS: (System 4) MH⁺=275, t_{RET}=2.66 min.

### Intermediate 18: Ethyl 2-({5-chloro-2-[(phenylmethyl)oxy]phenyl}methyl)-2H-1,2,3-triazole-4-carboxylate

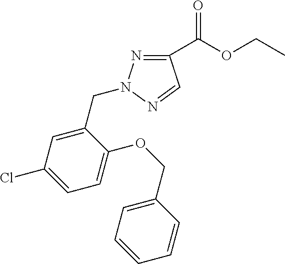

To a solution of 1H-1,2,3-triazole-4-carboxylic acid, ethyl ester (0.277 g, 1.96 mmol, Chemgenx) and 2-(bromomethyl)-4-chloro-1-[(phenylmethyl)oxy]benzene (0.617 g, 1.98 mmol, synthesised according to WO 200606696) in DMF (5 ml) was added potassium carbonate (0.682 g, 4.93 mmol). The suspension was stirred at ambient temperature under nitrogen overnight. The suspension was partitioned between ethyl acetate (50 ml) and water (50 ml). The phases were separated and the aqueous phase washed with ethyl acetate (50 ml). The combined organic extracts were washed with brine (25 ml) and the solvent removed in vacuo. The residue was loaded in dichloromethane and purified on silica (Si) 100 g using 0-50% ethyl acetate-cyclohexane. The appropriate fractions were combined and evaporated in vacuo to give the title compound as a waxy solid (0.232 g); LCMS: (System 1) MH⁺=372, t_{RET}=3.69 min.

### Intermediate 19: 2-({5-Chloro-2-[(phenylmethyl)oxy]phenyl}methyl)-2H-1,2,3-triazole-4-carboxylic acid

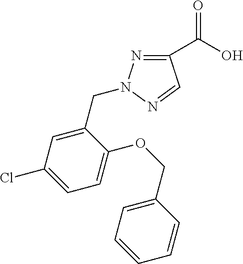

To a solution of ethyl 2-({5-chloro-2-[(phenylmethyl)oxy]phenyl}methyl)-2H-1,2,3-triazole-4-carboxylate (0.228 g, 0.613 mmol) in ethanol (3 ml) was added sodium hydroxide (0.74 ml, 1.47 mmol). The mixture was stirred at ambient temperature for 2.5 h. The solvent was removed in vacuo and the residue partitioned between dichloromethane (10 ml) and water (10 ml). The biphasic mixture was acidified using 2 M aqueous HCl (2 ml). The phases were separated using a hydrophobic frit and the aqueous phase washed with dichloromethane (2×10 ml). The combined organic extracts were concentrated in vacuo to leave the title compound as a colourless gum (210 mg); LCMS: (System 1) (M-H)⁻=342, t_{RET}=3.40 min.

### Intermediate 20: {2-[(Cyclopropylmethyl)oxy]phenyl}methanol

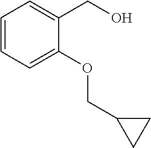

A mixture of 2-(hydroxymethyl)phenol (2 g, 16.1 mmol, Aldrich), (bromomethyl)cyclopropane (2.18 g, 16.1 mmol, Aldrich), and potassium carbonate (4.45 g, 32.2 mmol) in anhydrous N,N-dimethylformamide (10 ml) was stirred at 80° C. overnight. The reaction mixture was filtered and evaporated in vacuo. The residue was partitioned between water (40 ml) and ethylacetate (40 ml). The aqueous phase was extracted with further ethyl acetate (40 ml) and the combined organic extracts were evaporated in vacuo. The residue was diluted with DCM and flushed through a silica plug eluting with cyclohexane:EtOAc 10:1, 5:1, 2:1 (600 ml of each). Fractions containing product were combined and evaporated in vacuo to give the title compound as a pale yellow oil. (2.37 g); LCMS: (System 4) t_{RET}=2.26 min, no strong MH+ or (M-H)⁻; TLC: R_{f}=0.57 (cyclohexane:EtOAc 2:1).

### Intermediate 21: 1-(Bromomethyl)-2-[(cyclopropylmethyl)oxy]benzene

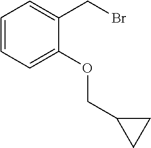

A solution of {2-[(cyclopropylmethyl)oxy]phenyl}methanol (0.5 g, 2.81 mmol) in anhydrous dichloromethane (DCM) (5 mL) was cooled to 5° C. under nitrogen. A solution of phosphorus tribromide (0.267 ml, 2.83 mmol) in anhydrous DCM (2 ml) was added dropwise and the reaction mixture stirred under nitrogen allowing to warm up to room temperature for 1 h. The reaction mixture was then dripped onto a mixture of ice/water (40 ml) and saturated aqueous sodium bicarbonate (15 ml), once the ice had melted the mixture was extracted with DCM (2×30 ml). The combined DCM extracts were evaporated in vacuo to give the title compound as a light brown oil (605 mg); LCMS: (System 4) t_{RET}=3.28 mins, no strong MH⁺ or (M-H)⁻; TLC: R_{f}=0.86 (cyclohexane:EtOAc 4:1).

### Intermediate 22: N-(2,6-difluorophenyl)-1H-1,2,3-triazole-4-carboxamide

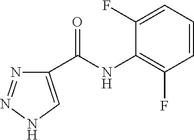

1.6 M butyllithium in hexanes, (62.8 ml, 100 mmol) was added dropwise to a solution of 2,6-difluoroaniline, (10.8 ml, 100 mmol, Fluorochem) in dry tetrahydrofuran (90 ml) at −78° C. under nitrogen. The resulting suspension was stirred at −78° C. for 15 min and then allowed to warm to 0° C. to give a clear solution. A solution of ethyl 1H-1,2,3-triazole-4-carboxylate, (4.726 g, 33.5 mmol, Chemgenx) in dry tetrahydrofuran (40 ml) was added to the reaction mixture at 0° C. and the resulting mixture was allowed to warm to room temperature. After 2 h at room temperature, ethanol (5 ml) was carefully added under nitrogen, and the reaction mixture was evaporated to dryness to give an oily residue. This residue was treated with water (30 ml) and the pH was adjusted to approximately 8 with 2 M hydrochloric acid. The resulting solution was extracted with chloroform:ethanol (5:1, 4×120 ml). The aqueous solution from the extraction (approximately 70 ml total volume which contains inorganic impurities) was purified in 5 ml aliquots by passage through 6 g Oasis cartridges (four cartridges with 5 ml of aqueous solution per cartridge were run in parallel). Each cartridge was first washed with ethanol (2 column volumes) followed by water (2 column volumes). The aqueous solution (5 ml) was then applied and the cartridge was eluted with water (1.5 column volumes) followed by ethanol (2 column volumes). Fractions containing the required product (as judged by the UV absorption at 254 nM) were combined and evaporated to give the title compound as a white solid (5.34 g); LCMS: (System 3) MH⁺=225, t_{RET}=0.61 min.

### Intermediate 23: {2-[(Cyclobutylmethyl)oxy]phenyl}methanol

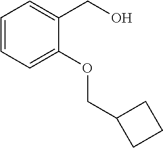

A mixture of 2-(hydroxymethyl)phenol (1 g, 8.06 mmol, Aldrich), (bromomethyl)cyclobutane (2.40 g, 16.1 mmol, Aldrich), potassium carbonate (2.23 g, 16.1 mmol) in anhydrous N,N-dimethylformamide (10 ml) was stirred at room temperature over the weekend then at 80° C. overnight. The reaction mixture was filtered and evaporated in vacuo. The residue was partitioned between water (20 ml) and ethyl acetate (20 ml). The aqueous phase was extracted with further ethyl acetate (20 ml), the combined ethyl acetate extracts were evaporated in vacuo. The sample was loaded in dichloromethane and purified by SPE on silica (Si) (70 g) using 0-50% ethyl acetate/cyclohexane. The appropriate fractions were combined and evaporated in vacuo to give the title compound as a colourless oil (1.54 g); LCMS: (System 3) t_{RET}=1.21 mins, no strong MH⁺ or (M-H)⁻; TLC: R_{f}=0.64 (cyclohexane:EtOAc 2:1).

### Intermediate 24: 1-(Bromomethyl)-2-[(cyclobutylmethyl)oxy]benzene

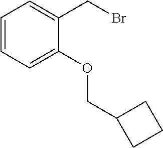

A solution of {2-[(cyclobutylmethyl)oxy]phenyl}methanol (0.5 g, 2.60 mmol) in anhydrous dichloromethane (5 ml) was cooled to 5° C. under nitrogen. A solution of phosphorus tribromide (0.248 ml, 2.63 mmol) in anhydrous DCM (2 ml) was added dropwise. The reaction mixture was stirred allowing to warm up to room temperature for 1.5 h. The reaction mixture was then poured onto a mixture of ice/water (40 ml) and saturated aqueous sodium bicarbonate (15 ml). DCM (30 ml) was added and the layers separated. The aqueous was extracted with DCM (20 ml). The combined DCM extracts were dried (Na₂SO₄) and evaporated in vacuo to give the title compound as a pale yellow oil (500 mg); LCMS: (System 4) t_{RET}=3.61 min, no strong MH⁺ or (M-H)⁻; R_{f}=0.91 in cyclohexane:EtOAc 4:1.

### Intermediate 25: Ethyl 2-[(2-iodophenyl)methyl]-2H-1,2,3-triazole-4-carboxylate

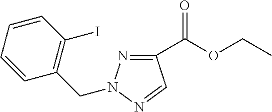

A solution of 1-(bromomethyl)-2-iodobenzene (4.21 g, 14.17 mmol, Aldrich) in dry dimethylformamide (10 ml) was added to a stirred solution of ethyl 1H-1,2,3-triazole-4-carboxylate (2 g, 14.17 mmol, Chemgenx) in dry dimethylformamide (25 ml) at room temperature. Potassium carbonate (3.92 g, 28.3 mmol) was added to the resulting solution and the resulting suspension was then stirred at room temperature for 4 h. The reaction mixture was filtered through a hydrophobic frit using DMF (5 ml) to wash the frit. The filtrate was evaporated to a residual oil which was dissolved in dichloromethane (300 ml) and washed with water (30 ml). After separation of the phases, the organic phase was dried over anhydrous sodium sulphate and evaporated to give a pale yellow oil (5.78 g). The oil was dissolved in dichloromethane (15 ml) and applied to three 100 g silica SPE cartridges. The cartridges were eluted using a gradient of 0 to 50% ethyl acetate in cyclohexane. Appropriate fractions were combined and evaporated to give the title compound as a colourless oil (1.20 g); LCMS: (System 3) MH⁺=358, t_{RET}=1.16 min.

### Intermediate 26: 2-[(2-Iodophenyl)methyl]-2H-1,2,3-triazole-4-carboxylic acid

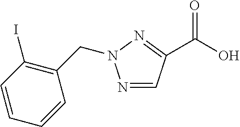

2 M aqueous sodium hydroxide solution (1.97 ml, 3.94 mmol) was added to a stirred mixture of ethyl 2-[(2-iodophenyl)methyl]-2H-1,2,3-triazole-4-carboxylate (1.174 g, 3.29 mmol) in ethanol (15 ml) and water (1 ml) and the resulting clear solution was stirred at room temperature for 1.5 h. The reaction mixture was evaporated to near dryness and the residual oil was dissolved in water (20 ml) and acidified to ˜pH 3 with 2 M aqueous hydrochloric acid. The resulting mixture was extracted with dichloromethane (100 ml+2×50 ml). The combined organic extracts were dried over anhydrous sodium sulphate and evaporated to give the title compound as a white solid (1.02 g); LCMS: (System 3) MH⁺=330, t_{RET}=0.90 min.

### Intermediate 27: N-(2,6-Difluorophenyl)-2-[(2-iodophenyl)methyl]-2H-1,2,3-triazole-4-carboxamide

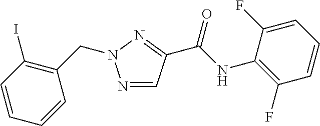

Thionyl chloride (10 ml, 137 mmol) was added to 2-[(2-iodophenyl)methyl]-2H-1,2,3-triazole-4-carboxylic acid (1.01 g, 3.06 mmol) and the resulting mixture was heated under gentle reflux (bath temperature at 90° C.) under nitrogen for 30 min. The reaction mixture was evaporated to dryness to give 2-[(2-iodophenyl)methyl]-2H-1,2,3-triazole-4-carbonyl chloride as a pale yellow oil (1.11 g).

2-[(2-iodophenyl)methyl]-2H-1,2,3-triazole-4-carbonyl chloride (1.06 g, assumed 3.06 mmol) was dissolved in dry dichloromethane (10 ml) and added, dropwise over 10 min, to a stirred solution of 2,6-difluoroaniline (0.494 ml, 4.59 mmol, Fluorochem) and triethylamine (1.71 ml, 12.24 mmol) in dry dichloromethane (15 ml) at room temperature for 1 h. The reaction mixture was heated at reflux (bath temperature at 53° C.), then cooled to room temperature and evaporated to a residual oil. This oil was dissolved in dry acetonitrile (20 ml) and treated with 2,6-difluoroaniline (2.47 ml, 23 mmol) and triethylamine (1.71 ml, 12.2 mmol). The resulting solution was heated under reflux (bath temperature at 95° C.) for 138 h. The reaction mixture was evaporated to dryness and the residual oil was distributed between dichloromethane (150 ml) and 0.5 M hydrochloric acid (25 ml). The phases were separated and the aqueous phase was extracted with further dichloromethane (50 ml). The combined organic extracts were dried over anhydrous sodium sulphate and evaporated to leave a pale brown oil (4.38 g). This oil was dissolved in dichloromethane (10 ml) and applied equally to two 100 g silica SPE cartridges. The cartridges were eluted using a gradient of 0 to 25% ethyl acetate in cyclohexane. Appropriate fractions were combined and evaporated to give the title compound as a pale brown solid (489 mg); LCMS: (System 3) MH⁺=441, t_{RET}=1.14 min.

### Intermediate 28: 2-[(5-Chloro-2-hydroxyphenyl)methyl]-N-(2,6-difluorophenyl)-2H-1,2,3-triazole-4-carboxamide

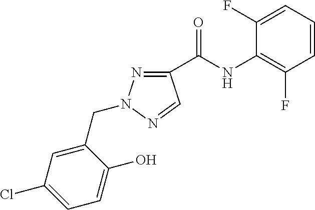

2-({5-chloro-2-[(phenylmethyl)oxy]phenyl}methyl)-N-(2,6-difluorophenyl)-2H-1,2,3-triazole-4-carboxamide (0.539 g, 1.185 mmol) was dissolved in ethyl acetate (59 ml) and hydrogenated using an H-cube with settings: 20° C., 1 bar, 1 ml/min flow rate) and 5% Pd/C CatCart 30 as the catalyst. The reaction mixture was evaporated to dryness then dissolved in ethyl acetate (25 ml) and hydrogenated conventionally over 5% palladium on carbon (50 mg) catalyst for 7 h. The reaction mixture was filtered through a 10 g celite cartridge and the celite cartridge was washed with ethyl acetate (25 ml). The filtrate and washings were combined and evaporated to give the title compound as a white foam (468 mg); LCMS: (System 3) MH⁺=365, 367, t_{RET}=1.01 min.

### Intermediate 29: 2-(Hydroxymethyl)-4-(trifluoromethyl)phenol

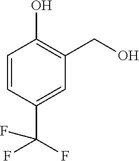

To a solution of 2-hydroxy-5-(trifluoromethyl)benzoic acid (synthesised according to Synthesis 2001, No. 15, 2259-2262, 3.13 g, 20.1 mmol) in anhydrous THF (65 ml) at 0° C. under nitrogen was added 1.0 M borane in THF (36 ml, 36 mmol, Aldrich) dropwise over 2 min. The mixture was allowed to warm to 20° C. and stirred over the weekend. To the mixture was slowly added water (75 ml) and then 2 M aqueous HCl (75 ml). The mixture was diluted with ethyl acetate (150 ml). The aqueous phase was further extracted with ethyl acetate. The combined organic extracts were dried (Na₂SO₄) and concentrated in vacuo to leave an oil. The residue was purified on silica (100 g) using a 0-100% DCM-ethyl acetate gradient over 40 min. The appropriate fractions were combined and the solvent removed in vacuo to give the title compound as a white solid (1.33 g); LCMS: (System 4) (M-H)−=191, t_{RET}=1.97 min.

### Intermediate 30: N-(2,6-difluorophenyl)-1-{[2-[(phenylmethyl)oxy]-5-(trifluoromethyl)phenyl]methyl}-1H-pyrazole-3-carboxamide

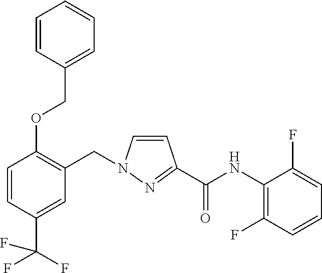

To a solution of 2-(hydroxymethyl)-4-(trifluoromethyl)phenol (2.19 g, 11.4 mmol) in acetone (50 ml) was added potassium carbonate (1.91 g, 13.8 mmol) and then benzyl bromide (1.49 g, 12.5 mmol, Aldrich). The mixture was stirred overnight at ambient temperature. The mixture was partitioned between water (200 ml) and ethyl acetate (200 ml). The phases were separated and the aqueous phase washed with ethyl acetate (200 ml). The combined organic extracts were dried (Na₂SO₄), filtered and the solvent removed in vacuo to leave 2-[(phenylmethyl)oxy]-5-(trifluoromethyl)phenyl]methanol as white solid (3.23 g, 100%).

To a stirred solution of 2-[(phenylmethyl)oxy]-5-(trifluoromethyl)phenyl]methanol (3.23 g, 11.4 mmol) in dichloromethane (60 ml) at ambient temperature was added triphenylphosphine (3.63 g, 13.8 mmol, Aldrich). To the solution was added carbon tetrabromide (4.59 g, 13.8 mmol, Aldrich) portionwise, maintaining <10° C. increase over ambient temperature. The mixture was stirred for 1.5 h. The solvent was evaporated in vacuo and the residue purified on silica (100 g) using a 0-50% cyclohexane-DCM gradient over 40 min. The appropriate fractions were combined and the solvent removed in vacuo to give 2-(bromomethyl)-1-[(phenylmethyl)oxy]-4-(trifluoromethyl)benzene as a colourless oil (2.57 g).

To a solution of N-(2,6-difluorophenyl)-1H-pyrazole-3-carboxamide (0.679 g, 3.04 mmol) in N,N-dimethylformamide (20 ml) was added potassium carbonate (0.608 g, 4.40 mmol) and then 2-(bromomethyl)-1-[(phenylmethyl)oxy]-4-(trifluoromethyl)benzene (990 mg, 2.87 mmol) at ambient temperature. The suspension was stirring overnight. The mixture was partitioned between water (150 ml) and DCM (150 ml). The aqueous phase was washed with dichloromethane (15 ml). The combined organic extracts were dried (Na₂SO₄) and the solvent evaporated in vacuo to leave a colourless oil. The residue was loaded in dichloromethane and purified on silica (Si) 100 g using 0-50% cyclohexane-ethyl acetate. The appropriate fractions were combined and the solvent removed in vacuo to leave a colourless oil. The semi-crude product was dissolved in ethyl acetate (100 ml) and washed with water (3×50 ml). The organic extract was dried (Na₂SO₄), filtered and the solvent removed in vacuo. The residue was dissolved in DCM and the solvent removed in vacuo twice to give the title compound as a white foam (1.20 g); LCMS: (System 4) MH⁺=488, t_{RET}=3.38 min.

### Intermediate 31: N-(2,6-difluorophenyl)-1-{[2-hydroxy-5-(trifluoromethyl)phenyl]methyl}-1H-pyrazole-3-carboxamide

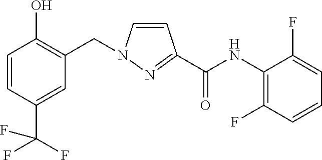

To 10% palladium on carbon (149 mg) under vacuum was added a solution of N-(2,6-difluorophenyl)-1-{[2-[(phenylmethyl)oxy]-5-(trifluoromethyl)phenyl]methyl}-1H-pyrazole-3-carboxamide (1.17 g, 2.41 mmol) in ethyl acetate (20 ml). The suspension was hydrogenated at ambient temperature for 3 h. The suspension was filtered through a celite cartridge (10 g) and the residue washed with ethyl acetate (70 ml). The solvent was removed in vacuo and the residue dissolved in methanol. The solvent was removed in vacuo to give the title compound a white powder (0.902 g); LCMS: (System 4) MH⁺=398, t_{RET}=2.72 min.

### Example 1

### 1-({5-Chloro-2-[(phenylmethyl)oxy]phenyl}methyl)-N-(2,6-difluorophenyl)-1H-pyrazole-3-carboxamide

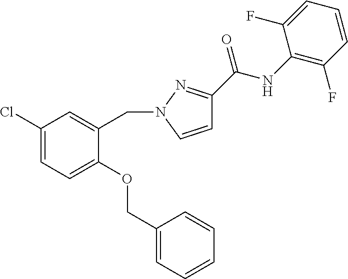

To 1-({5-chloro-2-[(phenylmethyl)oxy]phenyl}methyl)-1H-pyrazole-3-carboxylic acid (0.767 g, 2.24 mmol) was added thionyl chloride (6 ml, 82 mmol, Aldrich). The suspension was heated to reflux (drysyn block at 95° C.) for 2 h. The starting material dissolved on heating to reflux. The solvent was removed in vacuo and dried on a vacuum pump manifold to give 1-({5-chloro-2-[(phenylmethyl)oxy]phenyl}methyl)-1H-pyrazole-3-carbonyl chloride as a colourless gum (0.870 g). To a solution of 1-({5-chloro-2-[(phenylmethyl)oxy]phenyl}methyl)-1H-pyrazole-3-carbonyl chloride (0.7 g, 1.94 mmol) in DCM (8 ml) at ambient temperature was added triethylamine (0.270 ml, 1.94 mmol, Aldrich) and then 2,6-difluoroaniline (0.3 ml, 0.383 g, 2.97 mmol, Aldrich). The solution was stirred at ambient temperature overnight. The resulting suspension was partitioned between dichloromethane (50 ml) and saturated aqueous sodium hydrogen carbonate (50 ml). The phases were separated and the aqueous extract washed with dichloromethane (25 ml). The combined organic extracts were washed with saturated aqueous sodium hydrogen carbonate (25 ml), dried (MgSO₄), filtered and the solvent removed in vacuo. The residue was loaded in dichloromethane and purified on silica (Si) 70 g using 0-50% ethyl acetate-cyclohexane. The appropriate fractions were combined and evaporated in vacuo to give the title compound as a white solid (0.791 g); LCMS: (System 1) MH⁺=454, t_{RET}=3.60 min.

Similarly prepared was:

| | | | LCMS | |
|---|---|---|---|---|
| | | | Retention | |
| | | | Time | m/z |
| Example | Structure | Name | (mins) | (MH)+ |
| 48 | 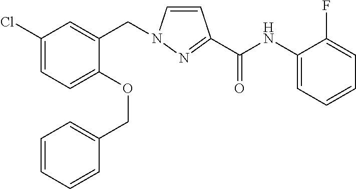 | 1-({5-chloro-2- [(phenylmethyl)oxy] phenyl}methyl)-N- (2-fluorophenyl)- 1H-pyrazole-3- carboxamide | 3.87 (System 1) | 436/438 |

### Example 2

### 1-({5-Chloro-2-[(2-methylpropyl)oxy]phenyl}methyl)-N-(2,6-difluorophenyl)-1H-pyrazole-3-carboxamide

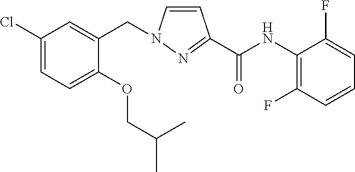

To a solution of 1-[(5-chloro-2-hydroxyphenyl)methyl]-N-(2,6-difluorophenyl)-1H-pyrazole-3-carboxamide (30 mg, 0.082 mmol) in DMF (1 ml) was added potassium carbonate (12-14 mg, 0.087 mmol). To the suspension was added 1-bromo-2-methyl-propane (0.012 ml, 0.107 mmol, Aldrich). The suspension was stirred at ambient temperature overnight. A further amount of potassium carbonate (12-14 mg, 0.087 mmol) and then 1-bromo-2-methyl-propane (0.024 ml, 0.21 mmol) was added to the suspension. The mixture was stirred overnight. The suspension was applied to a 10 g reversed phase C18 cartridge (pre-washed with methanol), using a small amount of methanol (0.5 ml) to load the compound. The cartridge was washed with 10% methanol in water (2 column volumes) and then methanol (2 column volumes). The methanol fraction was concentrated in vacuo. The residue was dissolved in 1:1 MeOH:DMSO (1 ml) and purified by MDAP (supelcosil ABZ+Plus column) (Method A) eluting with solvents A/B (A: Water+0.1% Formic acid, B:MeCN:Water 95:5+0.05% Formic acid). The solvent was evaporated in vacuo to give the title compound as a light brown solid (10 mg); LCMS: (System 1) MH⁺=420, t_{RET}=3.65 min.

### Example 3

### 1-({5-Chloro-2-[(3-methylbutyl)oxy]phenyl}methyl)-N-(2,6-difluorophenyl)-1H-pyrazole-3-carboxamide

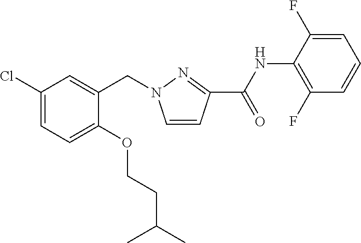

To a solution of 1-[(5-chloro-2-hydroxyphenyl)methyl]-N-(2,6-difluorophenyl)-1H-pyrazole-3-carboxamide (30 mg, 0.082 mmol) in DMF (0.5 ml) was added potassium carbonate (13-16 mg, 0.094 mmol). To the suspension was added 1-bromo-3-methylbutane (0.015 ml, 0.124 mmol, Aldrich) and the mixture stirred at ambient temperature overnight. The suspension was diluted with methanol (0.5 ml) and filtered through a cotton wool plug in a pasteur pipette directly into an MDAP vial. The filtrate was purified by MDAP (supelcosil ABZ+Plus column) (Method A) eluting with solvents A/B (A: Water+0.1% Formic acid, B:MeCN:Water 95:5+0.05% Formic acid). The solvent was evaporated in vacuo to give the title compound (18 mg); LCMS: (System 1) MH⁺=434, t_{RET}=3.75 min.

### Example 4

### 1-{[2-(Butyloxy)-5-chlorophenyl]methyl}-N-(2,6-difluorophenyl)-1H-pyrazole-3-carboxamide

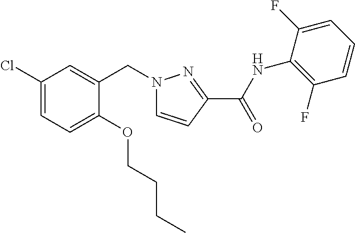

To a solution of 1-[(5-chloro-2-hydroxyphenyl)methyl]-N-(2,6-difluorophenyl)-1H-pyrazole-3-carboxamide (30 mg, 0.082 mmol) in DMF (0.5 ml) was added potassium carbonate (13-16 mg, 0.094 mmol). To the suspension was added 1-bromobutane (0.013 ml, 0.124 mmol, Acros) and the mixture stirred at ambient temperature overnight. The suspension was diluted with methanol (0.5 ml) and filtered through a cotton wool plug in a pasteur pipette directly into an MDAP vial. The filtrate was purified by MDAP (supelcosil ABZ+Plus column) (Method A) eluting with solvents A/B (A: Water+0.1% Formic acid, B:MeCN:Water 95:5+0.05% Formic acid). The solvent was evaporated in vacuo to give the title compound as a white solid (20 mg); LCMS: (System 1) MH⁺=420, t_{RET}=3.66 min.

### Example 5

### 1-({5-Chloro-2-[(cyclopropylmethyl)oxy]phenyl}methyl)-N-(2,6-difluorophenyl)-1H-pyrazole-3-carboxamide

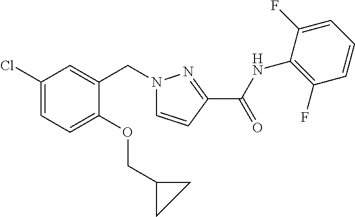

To a solution of 1-[(5-chloro-2-hydroxyphenyl)methyl]-N-(2,6-difluorophenyl)-1H-pyrazole-3-carboxamide (30 mg, 0.082 mmol) in DMF (0.5 ml) was added potassium carbonate (13-16 mg, 0.094 mmol). To the suspension was added (bromomethyl)cyclopropane (0.011 ml, 0.124 mmol, Alfa Aesar) and the mixture stirred at ambient temperature overnight. A further amount of potassium carbonate (13-16 mg, 0.094 mmol) and then (bromomethyl)cyclopropane (0.011 ml, 0.124 mmol, Alfa Aesar) was added to the suspension. The suspension was stirred at ambient temperature over the weekend. The suspension was diluted with methanol (0.5 ml) and filtered through a cotton wool plug in a pasteur pipette directly into an MDAP vial. The filtrate was purified by MDAP (supelcosil ABZ+Plus column) (Method A) eluting with solvents A/B (A: Water+0.1% Formic acid, B:MeCN:Water 95:5+0.05% Formic acid). The solvent was evaporated in vacuo to give the title compound as a white solid (18.6 mg); LCMS: (System 1) MH⁺=418, t_{RET}=3.54 min.

### Example 6

### 1-[(5-Chloro-2-{[(2,6-difluorophenyl)methyl]oxy}phenyl)methyl]-N-(2,6-difluorophenyl)-1H-pyrazole-3-carboxamide

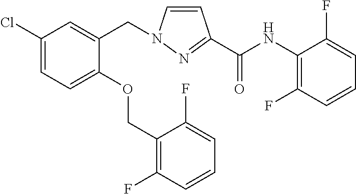

To a solution of 1-[(5-chloro-2-hydroxyphenyl)methyl]-N-(2,6-difluorophenyl)-1H-pyrazole-3-carboxamide (30 mg, 0.082 mmol) in anhydrous DMF (1 ml) was added potassium carbonate (13 mg, 0.094 mmol). To the suspension was added 2,6-difluorobenzyl bromide (23 mg, 0.111 mmol, Aldrich). The suspension was stirred overnight at ambient temperature. The suspension was applied to a 10 g reversed phase C18 cartridge (pre-washed with methanol), using a small amount of methanol (0.5 ml) to load the compound. The cartridge was washed with 10% methanol in water (2 column volumes) and then methanol (2 column volumes). The methanol fraction was concentrated in vacuo. The residue was dissolved in 1:1 MeOH:DMSO (1 ml) and purified by MDAP (supelcosil ABZ+Plus column) (Method A) eluting with solvents A/B (A: Water+0.1% Formic acid, B:MeCN:Water 95:5+0.05% Formic acid). The solvent was evaporated in vacuo to give the title compound as a white solid (22 mg); LCMS: (System 1) MH⁺=490, t_{RET}=3.62 min.

### Example 7

### 1-{[5-Chloro-2-(propyloxy)phenyl]methyl}-N-(2,6-difluorophenyl)-1H-pyrazole-3-carboxamide

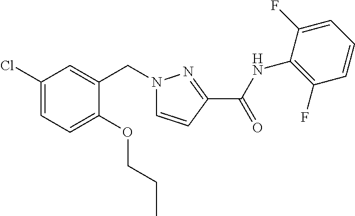

To a solution of 1-[(5-chloro-2-hydroxyphenyl)methyl]-N-(2,6-difluorophenyl)-1H-pyrazole-3-carboxamide (30 mg, 0.082 mmol) in DMF (0.5 ml) was added potassium carbonate (13-16 mg, 0.094 mmol). To the suspension was added 1-bromopropane (0.014 ml, 0.124 mmol, Aldrich) and the mixture stirred at ambient temperature overnight. The suspension was diluted with methanol (0.5 ml) and filtered through a cotton wool plug in a pasteur pipette directly into an MDAP vial. The filtrate was purified by MDAP (supelcosil ABZ+Plus column) (Method A) eluting with solvents A/B (A: Water+0.1% Formic acid, B:MeCN:Water 95:5+0.05% Formic acid). The solvent was evaporated in vacuo to give the title compound as a white solid (14.8 mg); LCMS: (System 1) MH⁺=406, t_{RET}=3.55 min.

### Example 8

### 1-({5-Chloro-2-[(cyclobutylmethyl)oxy]phenyl}methyl)-N-(2,6-difluorophenyl)-1H-pyrazole-3-carboxamide

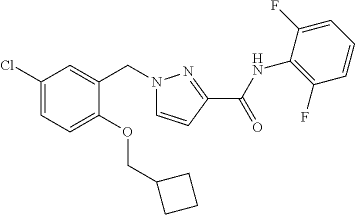

To a mixture of 1-[(5-chloro-2-hydroxyphenyl)methyl]-N-(2,6-difluorophenyl)-1H-pyrazole-3-carboxamide (41 mg, 0.113 mmol), and potassium carbonate (25 mg, 0.181 mmol) in DMF (0.5 ml) was added (bromomethyl)cyclobutane (0.016 ml, 0.143 mmol, Acros). The reaction mixture was stirred overnight, at ambient temperature under nitrogen. To the reaction mixture was added a further amount of (bromomethyl)cyclobutane (0.016 ml, 0.143 mmol, Acros) and the reaction stirred for 4 h. The reaction mixture was filtered using a hydrophobic frit and the filtrate dissolved in MeOH (0.5 ml). The filtrate was purified by MDAP (supelcosil ABZ+Plus column) (Method A) eluting with solvents A/B (A: Water+0.1% Formic acid, B:MeCN:Water 95:5+0.05% Formic acid). The solvent was evaporated in vacuo to give the title compound as a yellow gum (24.6 mg); LCMS: (System 1) MH⁺=432, t_{RET}=3.80 min.

### Example 9

### 1-[(5-Chloro-2-{[(2-chlorophenyl)methyl]oxy}phenyl)methyl]-N-(2,6-difluorophenyl)-1H-pyrazole-3-carboxamide

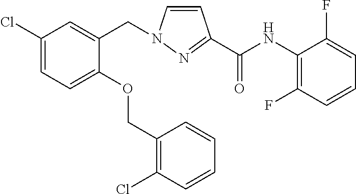

To a solution of 1-[(5-chloro-2-hydroxyphenyl)methyl]-N-(2,6-difluorophenyl)-1H-pyrazole-3-carboxamide (30 mg, 0.082 mmol) in anhydrous DMF (1 ml) was added potassium carbonate (13 mg, 0.094 mmol). To the suspension was added 2-chlorobenzyl bromide (0.014 ml, 0.107 mmol, Acros). The suspension was stirred overnight at ambient temperature. The suspension was applied to a 10 g reversed phase C18 cartridge (pre-washed with methanol), using a small amount of methanol (0.5 ml) to load the compound. The cartridge was washed with 10% methanol in water (2 column volumes) and then methanol (2 column volumes). The methanol fraction was concentrated in vacuo. The residue was dissolved in 1:1 MeOH:DMSO (1 ml) and purified by MDAP (supelcosil ABZ+Plus column) (Method A) eluting with solvents A/B (A: Water+0.1% Formic acid, B:MeCN:Water 95:5+0.05% Formic acid). The solvent was evaporated in vacuo to give the title compound as a white solid (20 mg); LCMS: (System 1) MH⁺=488, t_{RET}=3.74 min.

### Example 10

### 1-{[5-Chloro-2-(cyclobutyloxy)phenyl]methyl}-N-(2,6-difluorophenyl)-1H-pyrazole-3-carboxamide

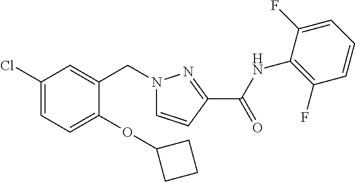

To a mixture of 1-[(5-chloro-2-hydroxyphenyl)methyl]-N-(2,6-difluorophenyl)-1H-pyrazole-3-carboxamide (41.5 mg, 0.114 mmol) and potassium carbonate (21.4 mg, 0.155 mmol) in DMF (0.5 ml) was added bromocyclobutane (0.014 ml, 0.148 mmol, Aldrich). The reaction was stirred overnight under nitrogen at ambient temperature. To the reaction was added a further amount of bromocyclobutane (0.028 ml, 0.296 mmol, Aldrich) and potassium carbonate (21 mg, 0.152 mmol). The reaction was stirred for a further 48 h, under nitrogen at ambient temperature. The reaction mixture was filtered using a hydrophobic frit and the filtrate dissolved in MeOH (0.5 ml). The filtrate was purified by MDAP (supelcosil ABZ+Plus column) (Method A) eluting with solvents A/B (A: Water+0.1% Formic acid, B:MeCN:Water 95:5+0.05% Formic acid). The solvent was evaporated in vacuo to give the title compound as a brown solid (3.6 mg); LCMS: (System 4) MH⁺=418, t_{RET}=3.38 min.

### Example 11

### 1-({5-Chloro-2-[(1-methylethyl)oxy]phenyl}methyl)-N-(2,6-difluorophenyl)-1H-pyrazole-3-carboxamide

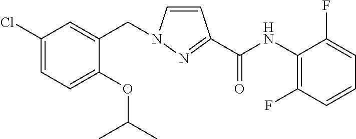

To a solution of 1-[(5-chloro-2-hydroxyphenyl)methyl]-N-(2,6-difluorophenyl)-1H-pyrazole-3-carboxamide (30 mg, 0.082 mmol) in DMF (1 ml) was added potassium carbonate (12-14 mg, 0.087 mmol). To the suspension was added 2-bromopropane (10 μl, 0.107 mmol, Aldrich). The suspension was stirred at ambient temperature overnight. A further amount of potassium carbonate (12-14 mg, 0.087 mmol) and then 2-bromopropane (20 μl, 0.21 mmol, Aldrich) was added to the suspension. The mixture was stirred overnight. The suspension was applied to a 10 g reversed phase C18 cartridge (pre-washed with methanol), using a small amount of methanol (0.5 ml) to load the compound. The cartridge was washed with 10% methanol in water (2 column volumes) and then methanol (2 column volumes). The methanol fraction was concentrated in vacuo. The residue was dissolved in 1:1 MeOH:DMSO (1 ml) and purified by MDAP (supelcosil ABZ+Plus column) (Method A) eluting with solvents A/B (A: Water+0.1% Formic acid, B:MeCN:Water 95:5+0.05% Formic acid). The solvent was evaporated in vacuo to give the title compound as a light brown solid (7.6 mg); LCMS: (System 1) MH⁺=406, t_{RET}=3.49 min.

### Example 12

### 1-({5-Chloro-2-[(cyclohexylmethyl)oxy]phenyl}methyl)-N-(2,6-difluorophenyl)-1H-pyrazole-3-carboxamide

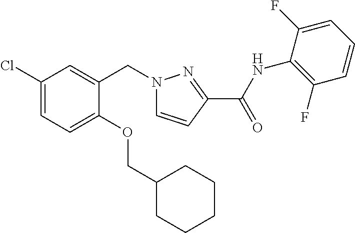

To a solution of 1-[(5-chloro-2-hydroxyphenyl)methyl]-N-(2,6-difluorophenyl)-1H-pyrazole-3-carboxamide (30 mg, 0.082 mmol) in DMF (0.5 ml) was added potassium carbonate (13-16 mg, 0.094 mmol). To the suspension was added (bromomethyl)cyclohexane (0.017 ml, 0.124 mmol, Aldrich) and the mixture stirred at ambient temperature overnight. A further amount of (bromomethyl)cyclohexane (0.017 ml, 0.124 mmol, Aldrich) and then potassium carbonate (13-16 mg, 0.094 mmol) was added to the suspension. The mixture was stirred over the weekend. The suspension was diluted with methanol (0.5 ml) and filtered through a cotton wool plug in a pasteur pipette. The filerate was purified by MDAP (supelcosil ABZ+Plus column) (Method A) eluting with solvents A/B (A: Water+0.1% Formic acid, B:MeCN:Water 95:5+0.05% Formic acid). The solvent was evaporated in vacuo to give the title compound as a colourless gum (5.2 mg); LCMS: (System 1) MH⁺=460, t_{RET}=3.90 min.

### Example 13

### 1-{[5-Chloro-2-(methyloxy)phenyl]methyl}-N-(2,6-difluorophenyl)-1H-pyrazole-3-carboxamide

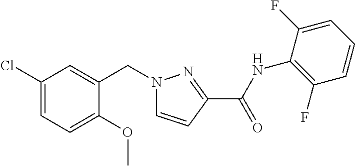

To a solution of 1-[(5-chloro-2-hydroxyphenyl)methyl]-N-(2,6-difluorophenyl)-1H-pyrazole-3-carboxamide (20 mg, 0.055 mmol) in DMF (1 ml) was added potassium carbonate (12.6 mg, 0.091 mmol) and then methyl iodide (5.3 μl, 0.085 mmol, Aldrich). The suspension was stirred overnight. The slight suspension was diluted with methanol (1 ml) and then purified by MDAP (supelcosil ABZ+Plus column) (Method A) eluting with solvents A/B (A: Water+0.1% Formic acid, B:MeCN:Water 95:5+0.05% Formic acid). The solvent was evaporated in vacuo to give the title compound as a yellow gum (13 mg); LCMS: (System 1) MH⁺=378, t_{RET}=3.30 min.

### Example 14

### 1-({5-Chloro-2-[(2-phenylethyl)oxy]phenyl}methyl)-N-(2,6-difluorophenyl)-1H-pyrazole-3-carboxamide

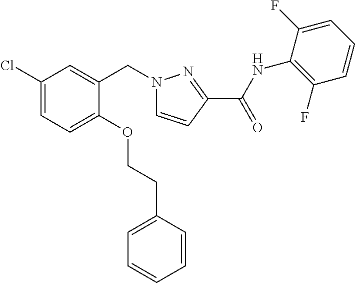

To a solution of 1-[(5-chloro-2-hydroxyphenyl)methyl]-N-(2,6-difluorophenyl)-1H-pyrazole-3-carboxamide (30 mg, 0.082 mmol) in DMF (0.5 ml) was added potassium carbonate (13-16 mg, 0.094 mmol). To the suspension was added (2-bromoethyl)benzene (0.017 ml, 0.124 mmol, Aldrich) and the mixture stirred at ambient temperature overnight. A further amount of (2-bromoethyl)benzene (0.017 ml, 0.124 mmol, Aldrich) and then potassium carbonate (13-16 mg, 0.094 mmol) was added to the suspension. The mixture was stirred over the weekend. The suspension was diluted with methanol (0.5 ml) and filtered through a cotton wool plug in a pasteur pipette. The filtrate was purified by MDAP (supelcosil ABZ+Plus column) (Method A) eluting with solvents A/B (A: Water+0.1% Formic acid, B:MeCN:Water 95:5+0.05% Formic acid). The solvent was evaporated in vacuo to give the title compound as white solid (3.8 mg); LCMS: (System 1) MH⁺=468, t_{RET}=3.69 min.

### Example 15

### N-(2,6-Difluorophenyl)-1-({2-[(phenylmethyl)oxy]phenyl}methyl)-1H-pyrazole-3-carboxamide

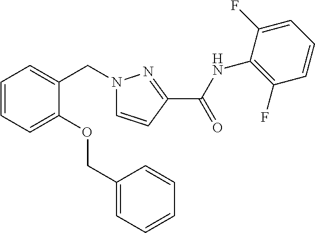

To a mixture of N-(2,6-difluorophenyl)-1H-pyrazole-3-carboxamide (0.510 g, 2.29 mmol) and potassium carbonate (0.70 g, 5.06 mmol) in DMF (20 ml) was added 2-benzyloxybenzylbromide (0.635 g, 2.29 mmol, Tiger). The reaction was stirred overnight, under nitrogen and at ambient temperature. The reaction mixture was partitioned between water (150 ml) and ethyl acetate (100 ml). The phases were separated and the aqueous phase extracted with ethyl acetate (100 ml). The combined organic phases were dried (MgSO₄), filtered and solvent removed in vacuo, to leave a yellow oil (2.32 g). The residue was loaded in dichloromethane and purified on silica (Si) 100 g using 0-100% ethyl acetate-cyclohexane. The appropriate fractions were combined and evaporated in vacuo to give the title compound as a colourless gum (0.879 g); LCMS: (System 1) MH⁺=420, t_{RET}=3.46 min.

### Example 16

### N-(2,6-Difluorophenyl)-1-({2-[(2-methylpropyl)oxy]phenyl}methyl)-1H-pyrazole-3-carboxamide

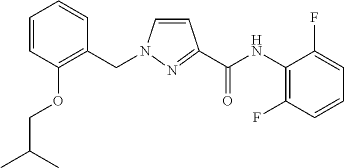

To a mixture of N-(2,6-difluorophenyl)-1-[(2-hydroxyphenyl)methyl]-1H-pyrazole-3-carboxamide (32.4 mg, 0.098 mmol) and potassium carbonate (17.2 mg, 0.124 mmol) in DMF (0.5 ml) was added 1-bromo-2-methylpropane (0.014 ml, 0.128 mmol, Aldrich). The reaction was stirred overnight, under nitrogen and at ambient temperature. The reaction mixture was filtered using a hydrophobic frit and the filtrate dissolved in MeOH (1 ml). The filtrate was purified by MDAP (supelcosil ABZ+Plus column) (Method A) eluting with solvents A/B (A: Water+0.1% Formic acid, B:MeCN:Water 95:5+0.05% Formic acid). The solvent was evaporated in vacuo to give the title compound as a clear gum (6.6 mg); LCMS: (System 1) MH⁺=386, t_{RET}=3.72 min.

### Example 17

### 1-({2-[(Cyclobutylmethyl)oxy]phenyl}methyl)-N-(2,6-difluorophenyl)-1H-pyrazole-3-carboxamide

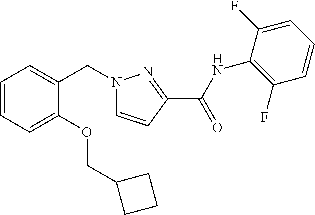

To a mixture of N-(2,6-difluorophenyl)-1-[(2-hydroxyphenyl)methyl]-1H-pyrazole-3-carboxamide (35.4 mg, 0.108 mmol) and potassium carbonate (42.1 mg, 0.305 mmol) in DMF (0.5 ml) was added (bromomethyl)cyclobutane (0.016 ml, 0.140 mmol, Acros). The reaction was stirred overnight under nitrogen and at ambient temperature. To the reaction mixture was added a further amount of (bromomethyl)cyclobutane (0.016 ml, 0.140 mmol, Acros) and potassium carbonate (18.3 mg, 0.133 mmol). The reaction was stirred overnight, under nitrogen and at ambient temperature. The reaction mixture was filtered using a hydrophobic frit and the filtrate dissolved in MeOH (0.5 ml). The filtrate was purified by MDAP (supelcosil ABZ+Plus column) (Method A) eluting with solvents A/B (A: Water+0.1% Formic acid, B:MeCN:Water 95:5+0.05% Formic acid). The solvent was evaporated in vacuo to give the title compound as a clear gum (22.2 mg);

LCMS: (System 4) MH⁺=398, t_{RET}=3.40 min.

### Example 18

### N-(2,6-Difluorophenyl)-1-({2-[(2-methylbutyl)oxy]phenyl}methyl)-1H-pyrazole-3-carboxamide

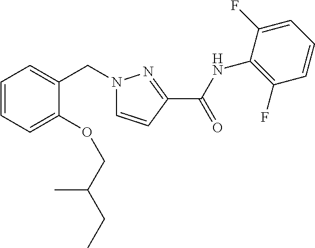

To a mixture of N-(2,6-difluorophenyl)-1-[(2-hydroxyphenyl)methyl]-1H-pyrazole-3-carboxamide (48 mg, 0.146 mmol) and potassium carbonate (29 mg, 0.210 mmol) in DMF (0.5 ml) was added 1-bromo-2-methylbutane (0.023 ml, 0.189 mmol, Friton Laboratories). The reaction was stirred overnight, under nitrogen and at ambient temperature. To the reaction mixture was added more 1-bromo-2-methylbutane (0.023 ml, 0.189 mmol) and potassium carbonate (29 mg, 0.224 mmol). The reaction was heated to 50° C. and stirred for 5 h under nitrogen. After cooling to ambient temperature, to the reaction mixture was added more 1-bromo-2-methylbutane (0.023 ml, 0.189 mmol). The reaction was stirred overnight, under nitrogen. The reaction mixture was filtered using a hydrophobic frit and the filtrate dissolved in MeOH (0.5 ml). The filtrate was purified by MDAP (Sunfire C18 column) (Method B). The solvent was evaporated in vacuo to give the title compound as white gum (15.3 mg); LCMS: (System 4) MH⁺=400, t_{RET}=3.50 min.

### Example 19

### N-(2,6-Difluorophenyl)-1-({2-[(3-methyl-2-buten-1-yl)oxy]phenyl}methyl)-1H-pyrazole-3-carboxamide

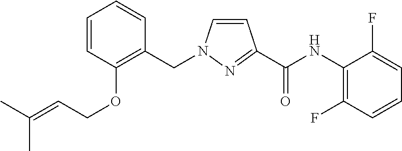

To a mixture of N-(2,6-difluorophenyl)-1-[(2-hydroxyphenyl)methyl]-1H-pyrazole-3-carboxamide (30.7 mg, 0.093 mmol) and potassium carbonate (19.5 mg, 0.141 mmol) in DMF (0.5 ml) was added 4-bromo-2-methyl-2-butene (0.014 ml, 0.121 mmol, Aldrich). The reaction was stirred overnight, under nitrogen and at ambient temperature. The reaction mixture was filtered using a hydrophobic frit and the filtrate dissolved in MeOH (1 ml). The filtrate was purified by MDAP (supelcosil ABZ+Plus column) (Method A) eluting with solvents A/B (A: Water+0.1% Formic acid, B:MeCN:Water 95:5+0.05% Formic acid). The solvent was evaporated in vacuo to give the title compound a brown gum (18.1 mg); LCMS: (System 1) MH⁺=398, t_{RET}=3.47 min.

### Example 20

### 1-{[2-(Cyclopentyloxy)phenyl]methyl}-N-(2,6-difluorophenyl)-1H-pyrazole-3-carboxamide

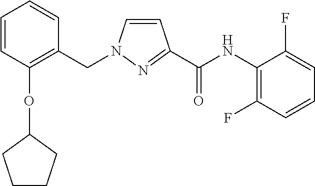

To a mixture of N-(2,6-difluorophenyl)-1-[(2-hydroxyphenyl)methyl]-1H-pyrazole-3-carboxamide (34 mg, 0.103 mmol) and potassium carbonate (21 mg, 0.152 mmol) in DMF (0.5 ml) was added cyclopentyliodide (0.016 ml, 0.138 mmol, Aldrich). The reaction mixture was stirred overnight under nitrogen and at ambient temperature. To the reaction mixture was added more potassium carbonate (18.2 mg, 0.132 mmol) and cyclopentyliodide (0.016 ml, 0.138 mmol, Aldrich). The reaction was stirred overnight, under nitrogen and at ambient temperature. The reaction mixture was filtered using a hydrophobic frit and the filtrate dissolved in MeOH (0.5 ml). The filtrate was purified by MDAP (supelcosil ABZ+Plus column) (Method A) eluting with solvents A/B (A: Water+0.1% Formic acid, B:MeCN:Water 95:5+0.05% Formic acid). The solvent was evaporated in vacuo to give the title compound as a pink solid (21.3 mg); LCMS: (System 1) MH⁺=398, t_{RET}=3.49 min.

### Example 21

### 1-{[2-(Cyclohexyloxy)phenyl]methyl}-N-(2,6-difluorophenyl)-1H-pyrazole-3-carboxamide

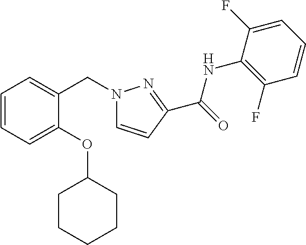

To a mixture of N-(2,6-difluorophenyl)-1-[(2-hydroxyphenyl)methyl]-1H-pyrazole-3-carboxamide (47 mg, 0.143 mmol) and potassium carbonate (28 mg, 0.20 mmol) in DMF (0.5 ml) was added iodocyclohexane (0.024 ml, 0.186 mmol, ABCR). The reaction was stirred overnight, at ambient temperature, under nitrogen. To the reaction mixture was added a further amount of iodocyclohexane (0.024 ml, 0.186 mmol, ABCR) and potassium carbonate (26 mg, 0.200 mmol). The reaction was heated to 50° C. and stirred overnight, under nitrogen. The reaction mixture was filtered using a hydrophobic frit and the filtrate dissolved in MeOH (0.5 ml). The filtrate was purified by MDAP (supelcosil ABZ+Plus column) (Method A) eluting with solvents A/B (A: Water+0.1% Formic acid, B:MeCN:Water 95:5+0.05% Formic acid). The solvent was evaporated in vacuo to give the title compound as an off-white gum (4.1 mg); LCMS: (System 4) MH⁺=412, t_{RET}=3.50 min.

### Example 22

### 1-{[5-Chloro-2-(phenyloxy)phenyl]methyl}-N-(2,6-difluorophenyl)-1H-pyrazole-3-carboxamide

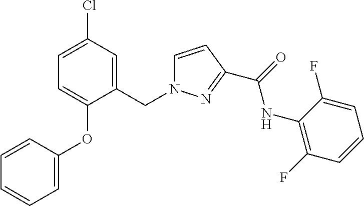

1-[(5-Chloro-2-iodophenyl)methyl]-N-(2,6-difluorophenyl)-1H-pyrazole-3-carboxamide (30 mg, 0.063 mmol), copper(I) bromide (0.91 mg, 6.33 μmol), cesium carbonate (43.3 mg, 0.133 mmol) and phenol (7.2 mg, 0.076 mmol, Aldrich) were weighed into a small test-tube. To this mixture was added DMSO (200 μL), followed by ethyl 2-oxocyclohexanecarboxylate (2.0 μL, 0.013 mmol, Aldrich). The test-tube was placed in a greenhouse apparatus and the contents were stirred and heated, under nitrogen, to 70° C. After 20 h the reaction solution was cooled then passed through a small plug of cotton wool and made up to 0.5 ml with 1:1 DMSO:MeOH solution. This sample was then purified by MDAP (supelcosil ABZ+Plus column) (Method A) eluting with solvents A/B (A: Water+0.1% Formic acid, B:MeCN:Water 95:5+0.05% Formic acid). The product containing fraction was passed through a polymer-supported bicarbonate cartridge and was evaporated to dryness to give the title compound as a white solid (7.6 mg); LCMS: (System 2) MH⁺=440 and 442 (01 isotopes), t_{RET}=1.33 min.

### Example 23

### 1-{[2-(Butyloxy)phenyl]methyl}-N-(2,6-difluorophenyl)-1H-pyrazole-3-carboxamide

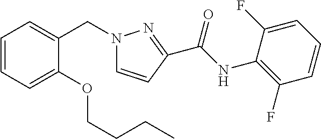

N-(2,6-Difluorophenyl)-1H-pyrazole-3-carboxamide (45 mg, 0.202 mmol) potassium carbonate (42 mg, 0.304 mmol) and 1-(bromomethyl)-2-(butyloxy)benzene (57 mg, 0.234 mmol) were weighed into a vial with a stirrer. DMF (0.5 ml) was added, the vial was capped and the mixture was stirred at room temperature for 26 h. The mixture was filtered through cotton wool, washing with MeOH to give a total volume of 1 ml. This solution was purified by MDAP (Sunfire column) (Method B). The appropriate fractions were concentrated in vacuo then under nitrogen flow to give the title compound as a colourless gum (23 mg); LCMS: (System 4) MH⁺=386, t_{RET}=3.31 min.

### Example 24

### 1-({5-Bromo-2-[(phenylmethyl)oxy]phenyl}methyl)-N-(2,6-difluorophenyl)-1H-pyrazole-3-carboxamide

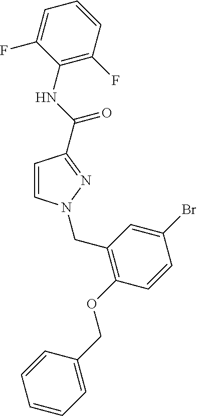

To a solution of N-(2,6-difluorophenyl)-1H-pyrazole-3-carboxamide (22.3 mg, 0.1 mmol) in dimethylformamide (0.4 ml) was added potassium t-butoxide (11.2 mg, 0.1 mmol) and the resulting solution left standing for 10 min. This solution was added to a solution of 4-bromo-2-(bromomethyl)phenyl phenylmethyl ether (0.1 mmol, synthesised according to WO 2005040128A1) in acetonitrile (0.2 ml). The resulting solution left standing at room temperature for 16 h. The solvent was removed in vacuo and a solution of 1:1 methanol:dimethylsulphoxide (0.6 ml) added and the solution purified by MDAP on SUNFIRE C18 column (Method D). The appropriate fraction was concentrated in vacuo then under nitrogen flow to give the title compound (18.9 mg); LCMS: (System 1) MH⁺=498, t_{RET}=3.76 min.

Similarly prepared were:

| | | | | |
|---|---|---|---|---|
| 25 | 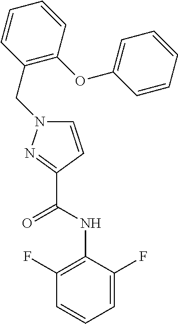 | N-(2,6- difluorophenyl)- 1-{[2- (phenyloxy) phenyl] methyl}-1H- pyrazole-3- carboxamide | 3.58 (System 1) | 406 |
| 26 | 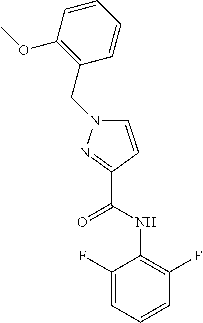 | N-(2,6- difluorophenyl)- 1-{[2- (methyloxy) phenyl] methyl}-1H- pyrazole-3- carboxamide | 1.07 (System 3) | 344 |

### Example 27

### 1-({4-Chloro-2-[phenylmethyl)oxy]phenyl}methyl)-N-(2,6-difluorophenyl)-1H-pyrazole-3-carboxamide

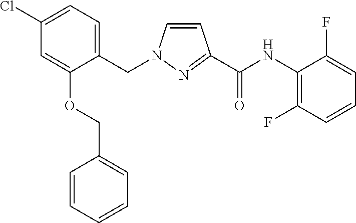

To a solution of {4-chloro-2-[(phenylmethyl)oxy]phenyl}methanol (0.501 g, 2.02 mmol) in DCM (10 ml) at −10 to −15° C., under nitrogen, was added a solution of phosphorus tribromide (0.190 ml, 2.02 mmol, Aldrich) in DCM (5 ml) dropwise. The solution was allowed to warm to ambient temperature and stirred for 4.5 h. To the reaction mixture in an ice-water bath was added dropwise saturated sodium hydrogen carbonate (5 ml). The mixture was diluted with water (45 ml) and dichloromethane (55 ml). The phases were separated and the aqueous phase washed with DCM (30 ml). The combined organic extracts washed with water (10 ml). The organic extract was dried (MgSO₄), filtered and the solvent removed in vacuo to give the 1-(bromomethyl)-4-chloro-2-[(phenylmethyl)oxy]benzene as a colourless gum (0.505 g).

To N-(2,6-difluorophenyl)-1H-pyrazole-3-carboxamide (50.9 mg, 0.228 mmol) and potassium carbonate (63.7 mg, 0.461 mmol) was added a solution of 1-(bromomethyl)-4-chloro-2-[(phenylmethyl)oxy]benzene (71.5 mg, 0.229 mmol) in DMF (1 ml). The reaction was stirred overnight, under nitrogen and at ambient temperature. The suspension was filtered using a hydrophobic frit and the residue washed with methanol (1 ml). The filtrate was purified by MDAP (supelcosil ABZ+Plus column) (Method A) eluting with solvents A/B (A: Water+0.1% Formic acid, B:MeCN:Water 95:5+0.05% Formic acid). The solvent was evaporated in vacuo to give the title compound as a light brown gum (60 mg); LCMS: (System 1) MH⁺=454, t_{RET}=3.70 min.

### Example 28

### 1-({2-[(3-Chlorophenyl)oxy]phenyl}methyl)-N-(2,6-difluorophenyl)-1H-pyrazole-3-carboxamide

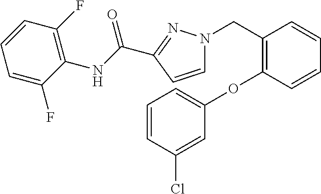

To a mixture of 1-[(2-bromophenyl)methyl]-N-(2,6-difluorophenyl)-1H-pyrazole-3-carboxamide (29 mg, 0.074 mmol), 3-chlorophenol (9.93 μl, 0.096 mmol, ABCR), cesium carbonate (50 mg, 0.153 mmol, Aldrich), ethyl-2-cyclohexanone-carboxylate (4.7 μl, 0.030 mmol, Aldrich) in DMSO (0.5 ml) was added copper (I) bromide-dimethyl sulfide complex (2.5 mg, 0.012 mmol, Aldrich). The reaction mixture was heated to 120° C. and stirred overnight under nitrogen. The reaction mixture was filtered using a hydrophobic frit and the filtrate dissolved in MeOH (0.5 ml). The sample was purified by MDAP (supelcosil ABZ+Plus column) (Method A) eluting with solvents A/B (A: Water+0.1% Formic acid, B:MeCN:Water 95:5+0.05% Formic acid). The solvent was evaporated in vacuo to give the title compound as a brown solid (8.9 mg); LCMS: (System 1) MH⁺=439.9, t_{RET}=3.62 min.

### Example 29

### 1-({2-[(4-Chlorophenyl)oxy]phenyl}methyl)-N-(2,6-difluorophenyl)-1 H-pyrazole-3-carboxamide

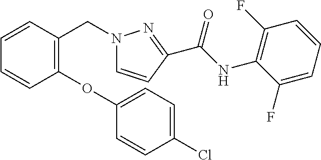

To a mixture of 1-[(2-bromophenyl)methyl]-N-(2,6-difluorophenyl)-1H-pyrazole-3-carboxamide (30 mg, 0.076 mmol), 4-chlorophenol (12 mg, 0.093 mmol, Aldrich), cesium carbonate (53 mg, 0.163 mmol, Aldrich), ethyl-2-cyclohexanone-carboxylate (4.9 μl, 0.031 mmol, Aldrich) in DMSO (0.5 ml) was added copper (I) bromide-dimethyl sulfide complex (3.5 mg, 0.017 mmol, Aldrich). The reaction was heated to 120° C. and stirred overnight under nitrogen. The reaction mixture was filtered using a hydrophobic frit and the filtrate dissolved in MeOH (0.5 ml). The sample was purified by MDAP (supelcosil ABZ+Plus column) (Method A) eluting with solvents A/B (A: Water+0.1% Formic acid, B:MeCN:Water 95:5+0.05% Formic acid). The solvent was evaporated in vacuo to give the title compound as a brown solid (6.8 mg); LCMS: (System 1) MH⁺=439.9, t_{RET}=3.63 min.

### Example 30

### N-(2,6-Difluorophenyl)-1-({2-[(4-fluorophenyl)oxy]phenyl}methyl)-1H-pyrazole-3-carboxamide

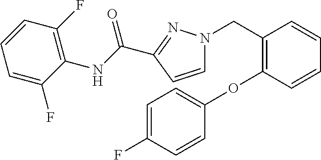

To a mixture of 1-[(2-bromophenyl)methyl]-N-(2,6-difluorophenyl)-1H-pyrazole-3-carboxamide (29 mg, 0.074 mmol), 4-fluorophenol (11 mg, 0.098 mmol, Aldrich), cesium carbonate (54 mg, 0.166 mmol, Aldrich) and ethyl-2-cyclohexanone-carboxylate (4.7 μL, 0.030 mmol, Aldrich) in DMSO (0.5 ml) was added copper (I) bromide-dimethyl sulfide complex (4 mg, 0.019 mmol, Aldrich). The reaction mixture was heated to 120° C. and stirred overnight under nitrogen. The reaction mixture was filtered using a hydrophobic frit and the filtrate dissolved in MeOH (0.5 ml). The sample was purified by MDAP (supelcosil ABZ+Plus column) (Method A) eluting with solvents A/B (A: Water+0.1% Formic acid, B:MeCN:Water 95:5+0.05% Formic acid). The solvent was evaporated in vacuo to give the title compound as a brown solid (6.1 mg); LCMS: (System 1) MH⁺=424, t_{RET}=3.50 min.

### Example 31

### N-(2,6-Difluorophenyl)-1-[(2-{[3-(methyloxy)phenyl]oxy}phenyl)methyl]-1H-pyrazole-3-carboxamide

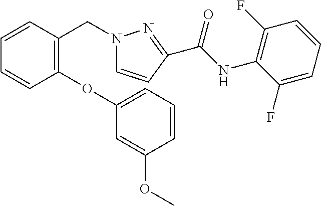

To a mixture of N-(2,6-difluorophenyl)-1-[(2-iodophenyl)methyl]-1H-pyrazole-3-carboxamide (39 mg, 0.089 mmol), 3-methoxyphenol (12.7 μl, 0.115 mmol, Aldrich), cesium carbonate (67 mg, 0.206 mmol, Aldrich), ethyl-2-cyclohexanone-carboxylate (5.8 μl, 0.036 mmol, Aldrich) in DMSO (0.5 ml) was added copper (I) bromide-dimethyl sulfide complex (6 mg, 0.029 mmol, Aldrich). The reaction was heated to 120° C. and stirred overnight, under nitrogen. The reaction mixture was filtered using a hydrophobic frit and the filtrate dissolved in MeOH (0.5 ml). The sample was purified by MDAP (supelcosil ABZ+Plus column) (Method A) eluting with solvents A/B (A: Water+0.1% Formic acid, B:MeCN:Water 95:5+0.05% Formic acid). The solvent was evaporated in vacuo to give the title compound as brown solid (10.4 mg); LCMS: (System 1) MH⁺=436, t_{RET}=3.48 min.

### Example 32

### N-(3,5-Difluoro-4-pyridinyl)-1-{[2-(phenyloxy)phenyl]methyl}-1H-pyrazole-3-carboxamide

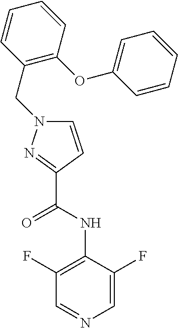

To 1-{[2-(phenyloxy)phenyl]methyl}-1H-pyrazole-3-carboxylic acid (0.76 g, 2.58 mmol) was added thionyl chloride (10 ml, 137 mmol, Aldrich) and the stirred mixture was heated to 90° C. under nitrogen for 4 h. Reaction was allowed to cool, then concentrated in vacuo to give the crude 1-{[2-(phenyloxy)phenyl]methyl}-1H-pyrazole-3-carbonyl chloride as a bright orange gum (837 mg).

To a solution of 3,5-difluoro-4-pyridinamine (6.5 mg, 0.05 mmol, Apollo) in N,N-dimethylformamide (0.1 ml) was added N,N-diisopropylethylamine (0.035 mL), followed by 1-{[2-(phenyloxy)phenyl]methyl}-1H-pyrazole-3-carbonyl chloride (15.6 mg, 0.05 mmol) in chloroform (0.1 ml). The resulting solution was shaken for 10 min and then left to stand at room temperature for a further 20 min. Dimethylsulphoxide (0.6 mL) was added and the solution purified by MDAP on SUNFIRE C18 column (Method D). The appropriate fraction was concentrated in vacuo then under nitrogen flow to give the title compound (1.2 mg); LCMS: (System 3) MH⁺=407, t_{RET}=1.15 min.

Similarly prepared were:

| | | | LCMS | |
|---|---|---|---|---|
| | | | Retention | |
| Ex- | | | Time | m/z |
| ample | Structure | Name | (mins) | (MH)+ |
| 33 | 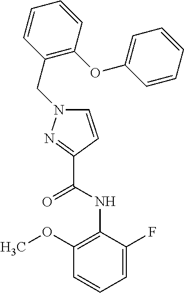 | N-[2- fluoro-6- (methyloxy) phenyl]-1- {[2-(phenyl- oxy)phenyl] methyl}-1H- pyrazole-3- carboxamide | 1.23 (System 3) | 418 |

### Example 34

### N-(2-fluorophenyl)-1-{[2-(phenyloxy)phenyl]methyl}-1H-pyrazole-3-carboxamide

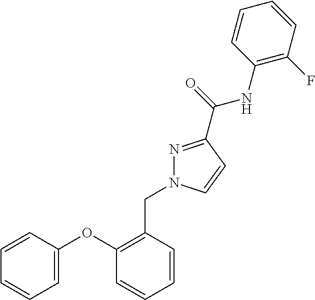

1-{[2-(Phenyloxy)phenyl]methyl}-1H-pyrazole-3-carboxylic acid (550 mg, 1.87 mmol) was treated with thionyl chloride (5 ml, 68.5 mmol, Aldrich) and DMF (0.01 ml, 0.13 mmol). The mixture was stirred at ambient temperature under nitrogen for 23 h. The reaction mixture was concentrated in vacuo, then redissolved in DCM and re-concentrated (×2) to give 1-{[2-(phenyloxy)phenyl]methyl}-1H-pyrazole-3-carbonyl chloride as a very pale yellow gum (579 mg).

To a solution of 2-fluoroaniline (22.2 mg, 0.2 mmol, Aldrich) in chloroform (0.2 ml) was added N,N-diisopropylethylamine (0.034 mL, 0.2 mmol) and dimethylaminopyridine (2 mg, 0.016 mmol) followed by 1-{[2-(phenyloxy)phenyl]methyl}-1H-pyrazole-3-carbonyl chloride (0.1 mmol) in chloroform (0.2 ml). The resulting solution was stirred and heated at 70° C. under reflux for 2 h, after which the chloroform was removed by evaporation. Dimethylsulphoxide (0.6 mL) was added and the solution purified by MDAP on SUNFIRE C18 column (Method D). The appropriate fraction was concentrated in vacuo then under nitrogen flow to give the title compound as a colourless solid; LCMS: (System 3) MH⁺=388, t_{RET}=1.36 min.

Similarly prepared was:

| | | | LCMS | |
|---|---|---|---|---|
| | | | Retention | |
| Ex- | | | Time | m/z |
| ample | Structure | Name | (mins) | (MH)+ |
| 35 | 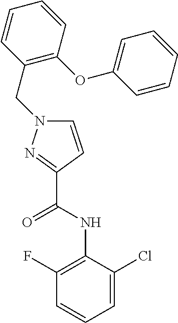 | N-(2-chloro- 6-fluoro- phenyl)-1- {[2-(phenyl- oxy)phenyl] methyl}-1H- pyrazole-3- carboxamide | 1.28 (System 3) | 422 |

### Example 36

### 1-{[2-(Butyloxy)phenyl]methyl}-N-(3,5-difluoro-4-pyridinyl)-1H-pyrazole-3-carboxamide

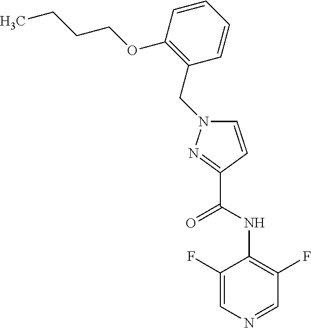

To 1-{[2-(butyloxy)phenyl]methyl}-1H-pyrazole-3-carboxylic acid (0.61 g, 2.22 mmol) was added thionyl chloride (10 ml, 137 mmol) followed by N,N-dimethylformamide (0.2 ml, 2.58 mmol). The reaction was stirred at ambient temperature under nitrogen. After 2 h, the reaction was concentrated in vacuo, dried in vacuo overnight, then redissolved in chloroform and reconcentrated (×2) and dried again in vacuo to give 1-{[2-(butyloxy)phenyl]methyl}-1H-pyrazole-3-carbonyl chloride (748 mg, 2.55 mmol). To a solution of 3,5-difluoro-4-pyridinamine (6.5 mg, 0.05 mmol, Apollo) in N,N-dimethylformamide (0.1 mL) was added N,N-diisopropylethylamine (0.035 ml), followed by 1-{[2-(butyloxy)phenyl]methyl}-1H-pyrazole-3-carbonyl chloride (14.6 mg, 0.05 mmol) in chloroform (0.1 ml). The resulting solution was shaken for 10 min and then left to stand at room temperature for a further 20 min. Dimethylsulphoxide (0.6 ml) was added and the solution purified by MDAP on SUNFIRE C18 column (Method D). The appropriate fraction was concentrated in vacuo then under nitrogen flow to give the title compound as a colourless solid (2.8 mg); LCMS: (System 3) MH⁺=387, t_{RET}=1.2 min.

Similarly prepared were:

| | | | LCMS | |
|---|---|---|---|---|
| | | | Retention | m/z |
| Example | Structure | Name | Time (mins) | (MH)+ |
| 37 | 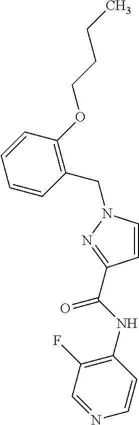 | 1-{[2-(butyloxy) phenyl]methyl}- N-(3-fluoro-4- pyridinyl)-1H- pyrazole-3- carboxamide | 1.23 (System 3) | 369 |
| 38 | 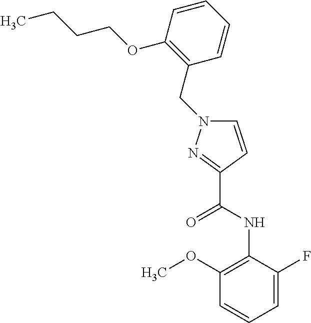 | 1-{[2-(butyloxy) phenyl]methyl}- N-[2-fluoro-6- (methyloxy)phenyl]- 1H-pyrazole-3- carboxamide | 1.28 (System 3) | 398 |

### Example 39

### 1-{[2-(Butyloxy)phenyl]methyl}-N-(2-chloro-6-fluorophenyl)-1H-pyrazole-3-carboxamide

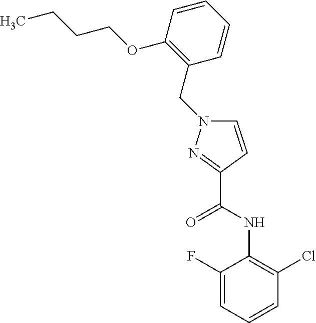

To 1-{[2-(butyloxy)phenyl]methyl}-1H-pyrazole-3-carboxylic acid (0.558 g, 2.03 mmol) was added thionyl chloride (5 ml, 68.5 mmol) followed by N,N-dimethylformamide (0.01 ml, 0.129 mmol). The reaction was stirred at ambient temperature under nitrogen for 22 h. The reaction mixture was concentrated in vacuo, then redissolved in DCM and re-concentrated (×2) to give 1-{[2-(butyloxy)phenyl]methyl}-1H-pyrazole-3-carbonyl chloride as a very pale orange gum which solidified on standing (611 mg). To a solution of 2-chloro-6-fluoroaniline (29.1 mg, 0.2 mmol, Avacardo research chemicals) in chloroform (0.2 ml) was added N,N-diisopropylethylamine (0.034 ml, 0.2 mmol) and dimethylaminopyridine (2 mg, 0.016 mmol). 1-{[2-(butyloxy)phenyl]methyl}-1H-pyrazole-3-carbonyl chloride (29.2 mg, 0.1 mmol) in chloroform (0.2 ml) was then added and the resulting solution stirred and heated at 70° C. under reflux for 2 h. The solvent was removed under a stream of nitrogen and then dimethylsulphoxide (0.6 ml) added. The reaction mixture was purified by MDAP on SUNFIRE C18 column (Method D). The appropriate fraction was concentrated in vacuo then under nitrogen flow to give the title compound (9.8 mg); LCMS: (System 3) MH⁺=402, t_{RET}=1.33 min.

### Example 40

### 2-({5-Chloro-2-[(phenylmethyl)oxy]phenyl}methyl)-N-(2,6-difluorophenyl)-2H-1,2,3-triazole-4-carboxamide

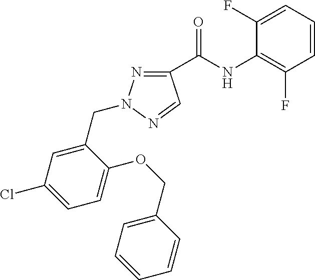

A suspension of 2-({5-chloro-2-[(phenylmethyl)oxy]phenyl}methyl)-2H-1,2,3-triazole-4-carboxylic acid (100 mg, 0.291 mmol) in thionyl chloride (1 ml, 13.7 mmol, Aldrich) was heated to 90° C. for 2.5 h. The solvent was removed in vacuo and dried under vacuum for 1 h. The residue was dissolved in DCM (1 ml). To the solution was added triethylamine (0.041 ml, 0.291 mmol) and then 2,6-difluoroaniline (0.044 ml, 0.436 mmol, Fluorochem). The solution was stirred at ambient temperature overnight. The suspension was diluted with methanol and applied to a 10 g SCX cartridge (pre-washed with methanol). The cartridge was washed with methanol (2 column volumes) and the methanol fraction concentrated in vacuo. The residue was loaded in dichloromethane and purified on silica (Si) 20 g using 0-50% ethyl acetate-cyclohexane. The appropriate fractions were combined and evaporated in vacuo. The residue was dissolved in 1:1 MeOH:DMSO (1 ml) and purified by MDAP (supelcosil ABZ+Plus column) (Method A) eluting with solvents A/B (A: Water+0.1% Formic acid, B:MeCN:Water 95:5+0.05% Formic acid). The solvent was evaporated in vacuo to give the title compound as a gum (34 mg); LCMS: (System 1) MH⁺=455, t_{RET}=3.59 min.

### Example 41

### 2-({2-[(Cyclopropylmethyl)oxy]phenyl}methyl)-N-(2,6-difluorophenyl)-2H-1,2,3-triazole-4-carboxamide

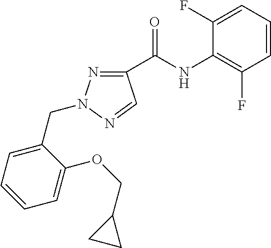

A mixture of 1-(bromomethyl)-2-[(cyclopropylmethyl)oxy]benzene (134 mg, 0.558 mmol), N-(2,6-difluorophenyl)-1H-1,2,3-triazole-4-carboxamide (125 mg, 0.558 mmol) and potassium carbonate (154 mg, 1.115 mmol) in anhydrous N,N-dimethylformamide (2.5 ml) was stirred at room temperature under nitrogen overnight. The reaction mixture was filtered and evaporated in vacuo. The sample was dissolved in DMSO (3×1 ml) and purified by Mass Directed AutoPrep on Sunfire C18 column using Acetonitrile Water with a Formic acid modifier (Method B). The solvent was evaporated in vacuo to give the title compound as a white solid (15 mg); LCMS: (System 4) MH⁺=385, t_{RET}=3.00 min.

### Example 42

### 2-({2-[(Cyclobutylmethyl)oxy]phenyl}methyl)-N-(2,6-difluorophenyl)-2H-1,2,3-triazole-4-carboxamide

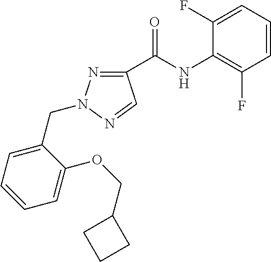

A mixture of 1-(bromomethyl)-2-[(cyclobutylmethyl)oxy]benzene (142 mg, 0.558 mmol), N-(2,6-difluorophenyl)-1H-1,2,3-triazole-4-carboxamide (125 mg, 0.558 mmol) and potassium carbonate (154 mg, 1.115 mmol) in anhydrous N,N-dimethylformamide (2.5 ml) was stirred at room temperature under nitrogen overnight. The reaction mixture was then filtered and evaporated in vacuo. The sample was dissolved in DMSO (3×1 ml) and purified by MDAP on Sunfire C18 column using Acetonitrile Water with a Formic acid modifier (Method B). The solvent was evaporated in vacuo to give the title compound as a colourless gum (27 mg); LCMS: (System 4) MH⁺=399, t_{RET}=3.26 min.

### Example 43

### N-(2,6-difluorophenyl)-2-{[2-(phenyloxy)phenyl]methyl}-2H-1,2,3-triazole-4-carboxamide

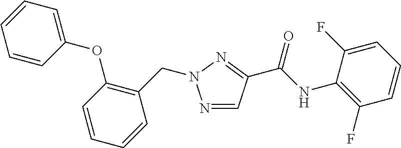

Dry dimethylsulphoxide (0.5 ml) was added to a mixture of copper (I) bromide (2.3 mg, 0.016 mmol), cesium carbonate (109 mg, 0.334 mmol) and ethyl 2-cyclohexanonecarboxylate (5.1 μl, 0.032 mmol, Aldrich) in a three-necked flask at room temperature under nitrogen. This mixture was stirred at room temperature for 30 min. A solution of N-(2,6-difluorophenyl)-2-[(2-iodophenyl)methyl]-2H-1,2,3-triazole-4-carboxamide (70 mg, 0.159 mmol) and phenol (18 mg, 0.191 mmol, Acros) in dry dimethylsulphoxide (0.5 ml) was added via syringe to the reaction mixture which was then heated at 80° C. under nitrogen for 21 h. The reaction mixture was filtered through a filter tube and the filtrate was diluted to a volume of 2 ml with dimethylsulphoxide-methanol (1:1). This solution was purified in two injections by MDAP (supelcosil ABZ+Plus column) (Method A) eluting with solvents A/B (A: Water+0.1% Formic acid, B:MeCN:Water 95:5+0.05% Formic acid). The solvent was evaporated in vacuo to give the title compound (17 mg) as a pale yellow oil; LCMS: (System 3) MH⁺=407, t_{RET}=1.22 min.

### Example 44

### 2-({5-chloro-2-[(2-methylpropyl)oxy]phenyl}methyl)-N-(2,6-difluorophenyl)-2H-1,2,3-triazole-4-carboxamide

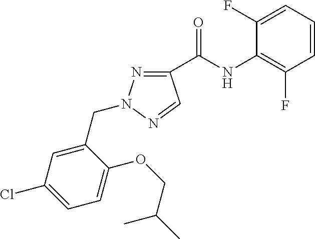

A 60% dispersion of sodium hydride in oil (24.2 mg, 0.605 mmol) was added portionwise, over 5 min, to a stirred solution of 2-[(5-chloro-2-hydroxyphenyl)methyl]-N-(2,6-difluorophenyl)-2H-1,2,3-triazole-4-carboxamide (210 mg, 0.576 mmol) in dry N,N-dimethylformamide (5 ml) at room temperature under nitrogen. The resulting mixture was stirred at room temperature for 10 min. Isobutyl bromide (0.069 ml, 0.633 mmol, Aldrich) was added to the reaction mixture which was maintained at room temperature under nitrogen for 23 h. The reaction mixture was heated at 50° C. for 5 h. The reaction mixture was allowed to cool to room temperature and then treated with tetrabutylammonium iodide (10 mg). After stirring at room temperature for 17 h a further quantity of isobutyl bromide (0.069 ml, 0.633 mmol, Aldrich) was added to the reaction mixture and stirring was continued at room temperature for 7 h. A further quantity of isobutyl bromide (0.069 ml, 0.633 mmol) was added and the reaction mixture was stirred at room temperature overnight. Isobutyl bromide (0.40 ml, 3.68 mmol) was added to the reaction mixture. After 3.5 h Isobutyl bromide (0.40 ml, 3.68 mmol) was added to the reaction mixture. After 2 h Isobutyl bromide (0.40 ml, 3.68 mmol) was added to the reaction mixture, and after 2 h a further quantity of isobutyl bromide (0.40 ml, 3.68 mmol) was added. The reaction mixture was left to stir at room temperature overnight. The reaction mixture was carefully treated with water (10 ml), acidified to pH-3 with 2 M hydrochloric acid and extracted with dichloromethane (3×25 ml). The combined organic extracts were dried over anhydrous sodium sulphate and evaporated to give a pale yellow oil (302 mg). The oil was dissolved in dichloromethane (3 ml) and applied to a 20 g silica SPE cartridge. The cartridge was eluted using a gradient of 0 to 50% ethyl acetate in cyclohexane. Appropriate fractions were combined and evaporated to give the title compound as a clear gum (79 mg); LCMS: (System 3) MH⁺=421, 423, t_{RET}=1.33 min.

### Example 45

### 2-{[2-(Butyloxy)-5-chlorophenyl]methyl}-N-(2,6-difluorophenyl)-2H-1,2,3-triazole-4-carboxamide

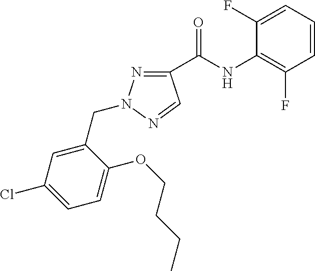

A 60% dispersion of sodium hydride in oil (33 mg, 0.825 mmol, Aldrich) was added portionwise, over 5 min, to a stirred solution of 2-[(5-chloro-2-hydroxyphenyl)methyl]-N-(2,6-difluorophenyl)-2H-1,2,3-triazole-4-carboxamide (300 mg, 0.823 mmol) in dry N,N-dimethylformamide (6 ml) at room temperature under nitrogen. The resulting mixture was stirred at room temperature for 10 min. 1-Bromobutane (0.098 ml, 0.905 mmol, Acros) was added to the reaction mixture which was maintained at room temperature under nitrogen for 22 h. The reaction mixture was carefully treated with water (10 ml), acidified to pH approximately 3 with 2 M hydrochloric acid and extracted with dichloromethane (3×25 ml). The combined organic extracts were dried over anhydrous sodium sulphate and evaporated to give a pale yellow oil (392 mg). The oil was dissolved in dichloromethane (5 ml) and applied to a 50 g silica SPE cartridge. The cartridge was eluted using a gradient of 0 to 50% ethyl acetate in cyclohexane. Appropriate fractions were combined and evaporated to a colourless oil (110 mg). The oil was dissolved in dichloromethane (2 ml) and applied to a 20 g silica SPE cartridge. The cartridge was eluted using a gradient of 0 to 25% ethyl acetate in cyclohexane. Appropriate fractions were combined and evaporated to give the title compound as a colourless oil which slowly crystallised to give a white crystalline solid (105 mg); LCMS: (System 3) MH⁺=421, 423, t_{RET}=1.32 min.

### Example 46

### 1-{[2-[(cyclobutylmethyl)oxy]-5-(trifluoromethyl)phenyl]methyl}-N-(2,6-difluorophenyl)-1H-pyrazole-3-carboxamide

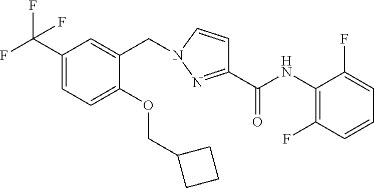

To a solution of N-(2,6-difluorophenyl)-1-{[2-hydroxy-5-(trifluoromethyl)phenyl]methyl}-1H-pyrazole-3-carboxamide (75 mg, 0.189 mmol) in dimethyl sulfoxide (1 ml) was added potassium carbonate (52 mg, 0.376 mmol) and then (bromomethyl)cyclobutane (0.030 ml, 0.268 mmol, Aldrich). The mixture was stirred at ambient temperature under nitrogen for 2 h. The solution was filtered using a hydrophobic frit and diluted with methanol (1 ml). The mixture was purified by MDAP on Sunfire C18 column using Acetonitrile Water with a Formic acid modifier (Method B). The solvent was evaporated in vacuo to give the title compound as a colourless gum (53 mg); LCMS: (System 4) MH⁺=466, t_{RET}=3.47 min.

### Example 47

### N-(2,6-difluorophenyl)-1-{[2-(ethyloxy)phenyl]methyl}-1H-pyrazole-3-carboxamide

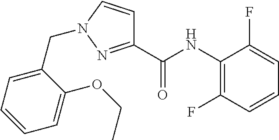

To a solution of 2-(ethyloxy)phenyl]methanol (0.315 g, 2.07 mmol, Acros) in DCM (8 ml) cooled in an ice-water bath under nitrogen was added dropwise phosphorus tribromide (0.196 ml, 2.08 mmol, Aldrich) in dichloromethane (2 ml). The mixture was allowed to warm to ambient temperature overnight. The reaction was re-cooled in an ice-water bath and then quenched with saturated sodium hydrogen carbonate solution (10 ml). The phases were separated and the aqueous phase extracted twice with DCM. The combined organic extracts were dried using a hydrophobic frit and the solvent removed in vacuo. The crude material was loaded in dichloromethane and purified on silica (Si) 100 g using 10% ethyl acetate in cyclohexane (100 ml) followed by 25% ethyl acetate in cyclohexane. The appropriate fractions were combined and evaporated in vacuo to give 1-(bromomethyl)-2-(ethyloxy)benzene as a colourless oil (0.393 g). To a mixture of N-(2,6-difluorophenyl)-1H-pyrazole-3-carboxamide (45 mg, 0.202 mmol) and potassium carbonate (56 mg, 0.405 mmol) was added a solution of 1-(bromomethyl)-2-(ethyloxy)benzene in DMF (0.5 ml). The mixture was stirred overnight at ambient temperature. The suspension was filtered through a cotton wool plug and the filterate diluted with methanol (1 ml). The filterate was purified by MDAP on Sunfire C18 column (Method B) using Acetonitrile Water with a Formic acid modifier. The solvent was evaporated using a stream of nitrogen to give the title compound as white solid (44 mg); LCMS: (System 4) MH⁺=358, t_{RET}=2.99 min.

### Example 49

### 1-{[2-({[3,5-bis(1,1-dimethylethyl)phenyl]methyl}oxy)phenyl]methyl}-N-(2,6-difluorophenyl)-1H-pyrazole-3-carboxamide

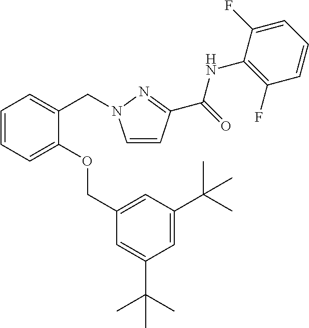

To a solution of N-(2,6-difluorophenyl)-1-[(2-hydroxyphenyl)methyl]-1H-pyrazole-3-carboxamide (33 mg, 0.1 mmol) and cesium carbonate (33 mg, 0.1 mmol) in DMF (0.4 ml) was added a solution of 3,5-di-t-butylbenzyl bromide (28 mg, 0.1 mmol, Aldrich) in DMF (0.1 ml). Reaction vessel was sealed and heated in microwave using initial 150 W to 100° C. for 10 min. The reaction mixture was dissolved in DMSO (0.5 ml) and purified by MDAP on Sunfire C18 column (Method D) using Acetonitrile-Water with a TFA modifier. The solvent was evaporated in vacuo using the Genevac to give the title compound (18 mg); LCMS: (System 3) MI-1⁺=532, t_{RET}=1.54 min.

Similarly prepared were:

| | | | LCMS | |
|---|---|---|---|---|
| | | | Retention | m/z |
| Example | Structure | Name | Time (mins) | (MH)+ |
| 50 | 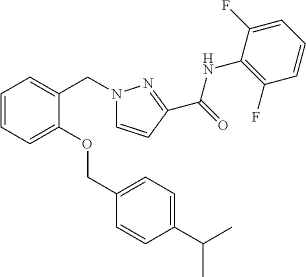 | N-(2,6-difluoro- phenyl)-1-{[2- ({[4-(1-methylethyl) phenyl]methyl}oxy) phenyl]methyl}-1H- pyrazole-3-carboxamide | 1.37 (System 3) | 462 |
| 51 | 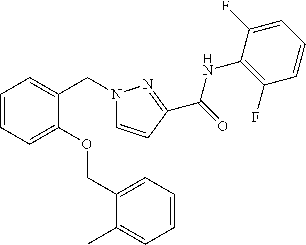 | N-(2,6-difluoro- phenyl)-1-[(2- {[(2-methylphenyl) methyl]oxy}phenyl) methyl]-1H-pyrazole- 3-carboxamide | 1.27 (System 3) | 434 |

### Example 52

### N-(2,6-difluorophenyl)-1-[(2-{[(4-methylphenyl)methyl]oxy}phenyl)methyl]-1H-pyrazole-3-carboxamide

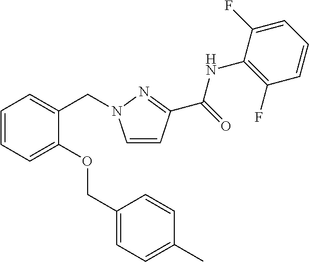

To a mixture of N-(2,6-difluorophenyl)-1-[(2-hydroxyphenyl)methyl]-1H-pyrazole-3-carboxamide (30 mg, 0.09 mmol) and cesium carbonate (33 mg, 0.1 mmol) in DMF (1 ml) was added 1-(bromomethyl)-4-methylbenzene (19 mg, 0.1 mmol, Alfa Aesar). The mixture was stirred at ambient temperature for 3 h. The reaction mixture was diluted with DCM (5 ml) and water (5 ml). The phases were separated and the aqueous layer was further extracted with DCM (5 ml). The combined organic extracts were dried through a hydrophobic frit and concentrated under a stream of nitrogen. The sample was dissolved in 1:1 MeOH:DMSO (1 ml) and purified by MDAP on Sunfire C18 column (Method B) using Acetonitrile-Water with a Formic acid modifier. The solvent was removed under a stream of nitrogen to give the title compound as a gum (32 mg); LCMS: (System 3) MH⁺=434, t_{RET}=1.32 min.

Similarly prepared were:

| | | | LCMS | |
|---|---|---|---|---|
| | | | Retention | m/z |
| Example | Structure | Name | Time (mins) | (MH)+ |
| 53 | 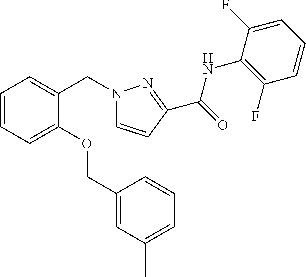 | N-(2,6-difluorophenyl)- 1-[(2-{[(3-methylphenyl) methyl]oxy}phenyl)methyl]- 1H-pyrazole-3-carboxamide | 1.31 (System 3) | 434 |
| 54 | 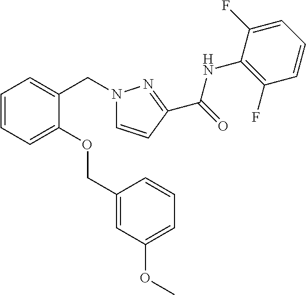 | N-(2,6-difluorophenyl)- 1-{[2-({[3-(methyloxy) phenyl]methyl}oxy)phenyl] methyl}-1H-pyrazole-3- carboxamide | 1.25 (System 3) | 450 |
| 55 | 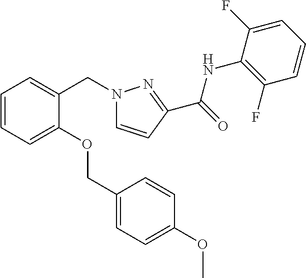 | N-(2,6-difluorophenyl)- 1-{[2-({[4-(methyloxy) phenyl]methyl}oxy) phenyl]methyl}-1H- pyrazole-3-carboxamide | 1.25 (System 3) | 450 |
| 56 | 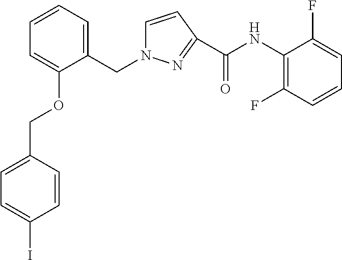 | N-(2,6-difluorophenyl)- 1-[(2-{[(4-iodophenyl) methyl]oxy}phenyl) methyl]-1H-pyrazole- 3-carboxamide | 1.35 (System 3) | 546 |
| 57 | 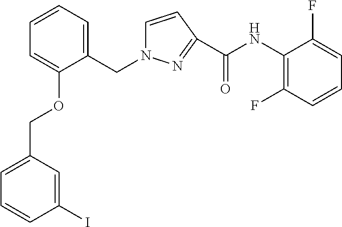 | N-(2,6-difluorophenyl)- 1-[(2-{[(3-iodophenyl) methyl]oxy}phenyl) methyl]-1H-pyrazole- 3-carboxamide | 1.33 (System 3) | 546 |

### Example 58

### N-(2,6-difluorophenyl)-1-{[2-({[2-(ethyloxy)phenyl]methyl}oxy)phenyl]methyl}-1H-pyrazole-3-carboxamide

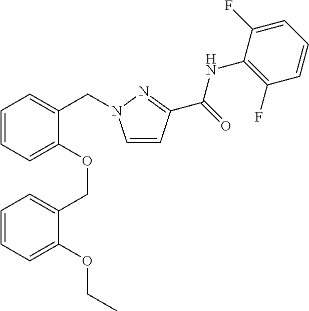

To a solution of N-(2,6-difluorophenyl)-1-[(2-hydroxyphenyl)methyl]-1H-pyrazole-3-carboxamide (30 mg, 0.09 mmol), triphenylphosphine (29 mg, 0.11 mmol, Aldrich) and 2-(ethyloxy)phenyl]methanol (17 mg, 0.1 mmol, Acros) in tetrahydrofuran (3 ml) at ambient temperature was added diisopropyl azodicarboxylate (0.027 ml, 0.14 mmol, Aldrich). The resulting mixture was stirred at ambient temperature for 3 h. The reaction mixture was diluted with DCM (5 ml) and water (5 ml). The phases were separated and the aqueous layer was further extracted with DCM (5 ml). The combined organic phases were dried through hydrophobic frit and concentrated under a stream of nitrogen. The samples were dissolved in 1:1 MeOH:DMSO (1 ml) and purified by MDAP on Sunfire C18 column (Method B) using Acetonitrile-Water with a Formic acid modifier. The solvent was evaporated in vacuo to give the title compound (34 mg); LCMS: (System 3) MH⁺=464, t_{RET}=1.32 min.

Similarly prepared were:

| | | | LCMS | |
|---|---|---|---|---|
| | | | Retention | m/z |
| Example | Structure | Name | Time (mins) | (MH)+ |
| 59 | 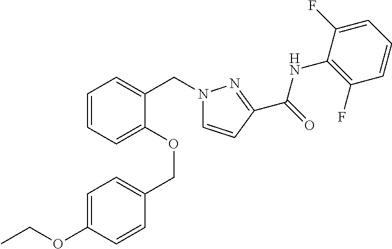 | N-(2,6-difluorophenyl)- 1-{[2-({[4-(ethyloxy) phenyl]methyl}oxy) phenyl]methyl}-1H- pyrazole-3-carboxamide | 1.28 (System 3) | 464 |
| 60 | 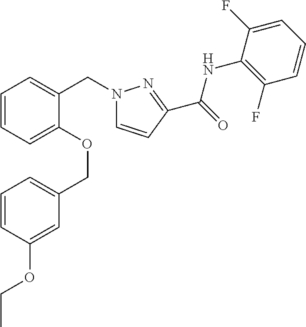 | N-(2,6-difluorophenyl)- 1-{[2-({[3-(ethyloxy) phenyl]methyl}oxy) phenyl]methyl}-1H- pyrazole-3-carboxamide | 1.29 (System 3) | 464 |

### Example 61

### N-(2,6-difluorophenyl)-1-{[2-({[3-(propyloxy)phenyl]methyl}oxy)phenyl]methyl}-1H-pyrazole-3-carboxamide

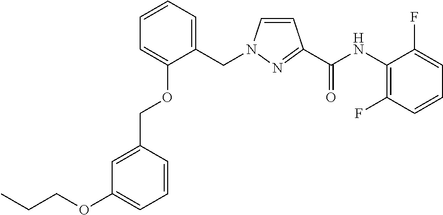

To a mixture of N-(2,6-difluorophenyl)-1-[(2-{[(3-iodophenyl)methyl]oxy}phenyl)methyl]-1H-pyrazole-3-carboxamide (16 mg, 0.03 mmol), cesium carbonate (20 mg, 0.06 mmol), copper(I) iodide (1.1 mg, 6 μmol, Alfa Aesar) and trans-9,10-dihydro-9,10-ethanoanthracene-11,12-dimethanol (1.6 mg, 6 μmol, Aldrich) was added 1-propanol (0.5 ml). The reaction vessel was sealed and heated in microwave using initial very high to 150° C. for 25 min. After cooling, the reaction mixture was partitioned between water (3 ml) and DCM (5 ml). The phases were separated using a hydrophobic frit and the organic phase concentrated in vacuo to leave the crude product (18 mg). The sample was dissolved in 1:1 MeOH:DMSO (1 ml) and purified by MDAP on Sunfire C18 column (Method B) using Acetonitrile-Water with a Formic acid modifier. The solvent was dried under a stream of nitrogen to give the title compound as a gum (3.2 mg); LCMS: (System 3) MH⁺=478, t_{RET}=1.38 min.

### Biological Experimental

The compounds can be tested according to the following or similar procedures.

This ICRAC assay uses the SERCA inhibitor thapsigargin to produce calcium depletion and activate an ICRAC current. The cells are incubated in a calcium-free environment, thus ionic movement does not occur until the calcium is added back and subsequently enters the cell via the open channels. Changes in intracellular calcium levels are determined by the inclusion of the calcium sensitive fluorescent dye Fluo-4 and the use of the FLIPR detection system. Inhibitors of ICRAC would be expected to decrease the calcium influx upon calcium add-back, thus reducing fluorescent signal.

Jurkat E6-1 is an established immortalised T lymphocyte cell line previously shown to express a functional ICRAC current. Jurkat cells grown in suspension are cultured in DMEM+10% FBS, maintained in T175 flasks at 37° C./5% CO₂, and are subcultured twice a week with either 1:10-1:20 splits. 1 confluent T175 yields 100 ml of approximately 2×10⁶ cells/ml.

Loading buffer contains; NaCl 145 mM, KCl 2.5 mM, HEPES 10 mM, Glucose 10 mM, MgCl₂ 1.2 mM, made up with water, then pH adjusted to 7.4 using NaOH 1 M. Finally Fluo-4AM & brilliant black are added to give a final assay concentration of 2 μM and 250 μM respectively.

Test buffer contains thapsigargin to give a final assay concentration of 5 μM, and test ICRAC inhibitor to give a final assay concentration of 15 μM to 14 μM. In instances of pIC50<4.8, compounds of the invention could be screened at a maximum concentration of either 50 μM or 150 μM

The required seeding density for a 384 plate is 20,000 cells per well. Cells are plated onto a 384 well plate, and loading buffer is added before being incubated at room temperature for 2.5 hours. Subsequently, test buffer is added to the cell plate and incubated at room temperature for a further 10 minutes. Plates are then transferred to the FLIPR which initially measures baseline fluorescence, followed by any increase in fluorescence evoked by the online addition of a 6 mM (1.2 mM FAC) calcium solution.

Fluorescence in the absence of ICRAC inhibitor gives a 100% maximal signal, and increasing concentrations of an ICRAC inhibitor result in a decreased fluorescence signal which is expressed as a percentage inhibition of maximal signal. From the concentration-response relationship, the concentration producing a 50% inhibition of the maximal signal (pIC₅₀) can then be determined.

In the above assay the compounds of the following examples were found to have a pIC₅₀≧6.0
1-8, 15-19, 22, 24, 25, 36, 39, 40, 42-45, 52, 54, 55, 59 and 60

In the above assay the compounds of the following examples were found to have a pIC₅₀≧5.3 and <6.0
9-14, 20, 21, 23, 27-35, 37, 38, 41, 46, 51, 53, 56, 58 and 61

In the above assay the compounds of the following examples were found to have a pIC₅₀<5.3
26, 47-50 and 57

All publications, including but not limited to patents and patent applications, cited in this specification are herein incorporated by reference as if each individual publication were specifically and individually indicated to be incorporated by reference herein as though fully set forth.

1. A compound of formula (I) or a salt thereof 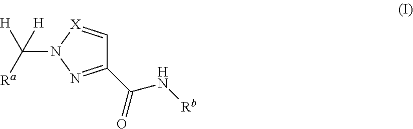
in which
X is CH or N;
R^{a} is a group of formula (a) 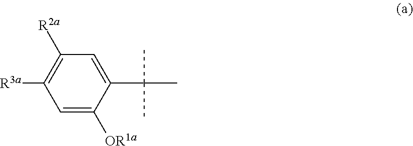
wherein
R^{1a} is a group —(CH₂)ₙ—R⁴
in which n is 0, 1, or 2; and
R⁴ is C₁₋₆alkyl, C₂₋₆alkenyl, C₃₋₇cycloalkyl or aryl;
R^{2a} and R^{1a} are independently H, halogen or CF₃;
R^{b} is a group of formula (b) 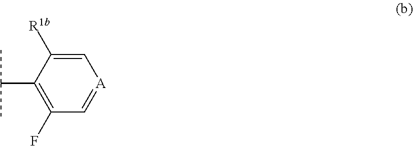
wherein
A is CH or N;
R^{1b} is H, halogen or C₁₋₆alkoxy.

2. A compound according to claim 1 in which X is CH.

3. A compound according to claim 1 in which R^{2a} is chloro and R^{3a} is H.

4. A compound according to claim 1 in which R^{2a} and R^{3a} are both H.

5. A compound according to claim 1 in which R^{1b} is H, fluoro, chloro or methoxy.

6. A compound according to claim 1 in which R^{b} is a group of formula (b) in which A is CH and R^{1b} is fluoro.

7. A compound selected from the group consisting of
1-({5-Chloro-2-[(phenylmethyl)oxy]phenyl}methyl)-N-(2,6-difluorophenyl)-1H-pyrazole-3-carboxamide;
1-({5-Chloro-2-[(2-methylpropyl)oxy]phenyl}methyl)-N-(2,6-difluorophenyl)-1H-pyrazole-3-carboxamide;
1-({5-Chloro-2-[(3-methylbutyl)oxy]phenyl}methyl)-N-(2,6-difluorophenyl)-1H-pyrazole-3-carboxamide;
1-{[2-(Butyloxy)-5-chlorophenyl]methyl}-N-(2,6-difluorophenyl)-1H-pyrazole-3-Carboxamide;
1-({5-Chloro-2-[(cyclopropylmethyl)oxy]phenyl}methyl)-N-(2,6-difluorophenyl)-1H-pyrazole-3-carboxamide;
1-[(5-Chloro-2-{[(2,6-difluorophenyl)methyl]oxy}phenyl)methyl]-N-(2,6-difluorophenyl)-1H-pyrazole-3-carboxamide;
1-{[5-Chloro-2-(propyloxy)phenyl]methyl}-N-(2,6-difluorophenyl)-1H-pyrazole-3-carboxamide;
1-({5-Chloro-2-[(cyclobutylmethyl)oxy]phenyl}methyl)-N-(2,6-difluorophenyl)-1H-pyrazole-3-carboxamide;
N-(2,6-Difluorophenyl)-1-({2-[(phenylmethyl)oxy]phenyl}methyl)-1H-pyrazole-3-carboxamide;
N-(2,6-Difluorophenyl)-1-({2-[(2-methylpropyl)oxy]phenyl}methyl)-1H-pyrazole-3-carboxamide;
1-({2-[(Cyclobutylmethyl)oxy]phenyl}methyl)-N-(2,6-difluorophenyl)-1H-pyrazole-3-carboxamide;
N-(2,6-Difluorophenyl)-1-({2-[(2-methylbutyl)oxy]phenyl}methyl)-1H-pyrazole-3-carboxamide;
N-(2,6-Difluorophenyl)-1-({2-[(3-methyl-2-buten-1-yl)oxy]phenyl}methyl)-1H-pyrazole-3-carboxamide;
1-{[5-Chloro-2-(phenyloxy)phenyl]methyl}-N-(2,6-difluorophenyl)-1H-pyrazole-3-carboxamide;
1-({5-Bromo-2-[(phenylmethyl)oxy]phenyl}methyl)-N-(2,6-difluorophenyl)-1H-pyrazole-3-carboxamide;
N-(2,6-difluorophenyl)-1-{[2-(phenyloxy)phenyl]methyl}-1H-pyrazole-3-carboxamide
1-{[2-(Butyloxy)phenyl]methyl}-N-(3,5-difluoro-4-pyridinyl)-1H-pyrazole-3-carboxamide;
1-{[2-(Butyloxy)phenyl]methyl}-N-(2-chloro-6-fluorophenyl)-1H-pyrazole-3-carboxamide;
2-({5-Chloro-2-[(phenylmethyl)oxy]phenyl}methyl)-N-(2,6-difluorophenyl)-2H-1,2,3-triazole-4-carboxamide;
2-({2-[(Cyclobutylmethyl)oxy]phenyl}methyl)-N-(2,6-difluorophenyl)-2H-1,2,3-triazole-4-carboxamide;
N-(2,6-difluorophenyl)-2-{[2-(phenyloxy)phenyl]methyl}-2H-1,2,3-triazole-4-carboxamide;
2-({5-chloro-2-[(2-methylpropyl)oxy]phenyl}methyl)-N-(2,6-difluorophenyl)-2H-1,2,3-triazole-4-carboxamide;
2-{[2-(Butyloxy)-5-chlorophenyl]methyl}-N-(2,6-difluorophenyl)-2H-1,2,3-triazole-4-carboxamide;
N-(2,6-difluorophenyl)-1-{[(2-[(4-methylphenyl)methyl]oxy}phenyl)methyl]-1H-pyrazole-3-carboxamide;
N-(2,6-difluorophenyl)-1-{[2-({[3-(methyloxy)phenyl]methyl}oxy)phenyl]methyl}-1H-pyrazole-3-carboxamide;
N-(2,6-difluorophenyl)-1-{[2-({[4-(methyloxy)phenyl]methyl}oxy)phenyl]methyl}-1H-pyrazole-3-carboxamide;
N-(2,6-difluorophenyl)-1-{[2-({[4-(ethyloxy)phenyl]methyl}oxy)phenyl]methyl}-1H-pyrazole-3-carboxamide; and
N-(2,6-difluorophenyl)-1-{[2-({[3-(ethyloxy)phenyl]methyl}oxy)phenyl]methyl}-1H-pyrazole-3-carboxamide
or a salt thereof.

8. (canceled)

9. (canceled)

10. (canceled)

11. A pharmaceutical composition comprising a compound claim 1 or a pharmaceutically acceptable salt thereof and one or more pharmaceutically acceptable carriers, diluents or excipients.

12. (canceled)

13. (canceled)

14. A method of treating asthma or rhinitis in a subject in need thereof which comprises administering a therapeutically effective amount of compound of formula (I) or a pharmaceutically acceptable salt thereof as defined in claim 1.
